(19) United States
(12) Patent Application Publication
(10) Pub. No.: US 2005/0059638 A1
(43) Pub. Date: Mar. 17, 2005

(54) Aryl, heteroaromatic and bicyclic aryl nitrone compounds, prodrugs and pharmaceutical compositions of the same to treat human disorders

(76) Inventors: Michael G. Kelly, Thousand Oaks, CA (US); Satyanarayana Janagani, Santa Clara, CA (US); Ravindra B. Upasani, San Jose, CA (US)

Correspondence Address: JONES DAY, 222 EAST 41ST ST, NEW YORK, NY, 10017, US

(21) Appl. No.: 10/911,788

(22) Filed: Aug. 3, 2004

Related U.S. Application Data
(60) Provisional application No. 60/492,488, filed on Aug. 4, 2003.
(60) Provisional application No. 60/492,489, filed on Aug. 4, 2003.
(60) Provisional application No. 60/492,490, filed on Aug. 4, 2003.

(51) Int. Cl. A61K 31/66; A61K 31/44; A61K 31/40; A61K 31/195

(52) U.S. Cl. 514/114; 514/357; 514/408; 514/561; 514/553; 514/644; 546/334; 548/571; 562/11; 562/66; 562/434; 564/296

(57) ABSTRACT

Disclosed are aryl, heteroaromatic and bicyclic aryl nitrone compounds and pharmaceutical compositions containing such derivatives. The disclosed compositions are useful for preventing and/or treating pain, neurodegenerative, autoimmune and inflammatory diseases or conditions in mammals.

This application claims priority under 35 U.S.C. § 119 to U.S. Provisional Application Nos. 60/492,488, 60/492,489 and 60/492,490, all filed on Aug. 4, 2003, the contents of which are hereby incorporated by reference in their entireties.

### 1. FIELD OF THE INVENTION

This invention relates to aryl, heteroaromatic and bicyclic aryl nitrone compounds and their use as therapeutic agents for the treatment of inflammation-related conditions in mammals such as (but not limited to) arthritis, neurodegenerative disorders such as (but not limited to) Parkinson's disease and Alzheimer's disease, stroke, uveitis, asthma, myocardial infarction, the treatment and prophylaxis of pain syndromes (acute and chronic or neuropathic), traumatic brain injury, acute spinal cord injury, alopecia (hair loss), inflammatory bowel disease and autoimmune disorders.

### 2. BACKGROUND OF THE INVENTION

Arthritis and related inflammatory disease conditions occur in more than 100 different forms, including rheumatoid arthritis (RA), osteoarthritis (OA), ankylosing spondylitis and systemic lupus erythematosus (SLE). Most forms of arthritis are characterized by some type of chronic inflammation. For example, RA typically involves chronic inflammation of the lining of the joints and/or the internal organs. Such chronic inflammation generally causes pain and swelling in the joints of those afflicted and may result in damage to cartilage, bone, tendons, ligaments and the like, ultimately leading to deformity and disability.

Prostaglandins (PG) have long been known to be involved in the inflammation process. Accordingly, a number of inhibitors of PG synthesis have been developed for the treatment of arthritis and related inflammatory disease conditions. Such non-steroidal anti-inflammatory drugs (NSAIDs) typically prevent the production of PGs by inhibiting enzymes such as cycloxygenase (COX) and lipoxygenase. The enzyme COX is known to exist in two forms. COX-1 is a constitutive form found in most tissues and organs. Among other properties, COX-1 produces small amounts of PGs necessary for maintaining the integrity of the GI track. COX-2 is an inducible form associated with the increased production of PGs during inflammatory conditions. Since many NSAIDs inhibit both forms of COX, they interfere with PG-regulated processes not associated with the inflammation process. As a result, many NSAIDs cause severe side effects, such as stomach ulcers and renal damage, which limit their effectiveness as therapeutics.

Accordingly, a need exists for novel classes of therapeutic compounds which effectively treat arthritis and other inflammatory-related conditions without producing undesirable side effects.

Nitrones constitute a class of compounds that have antioxidant properties due to their ability to form stable adducts (i.e., spin traps) with free radicals. Since free radicals can cause oxidative damage to cellular constituents (e.g., proteins and lipids), which can lead to pathological consequences, it has been reported that the antioxidant properties of nitrones at least partly underlie their therapeutic potential. Therefore, diseases which have been reported to be susceptible to antioxidant therapy or which involve the generation of free radicals may be susceptible to nitrone treatment based on the antioxidant activity of nitrones.

Aromatic nitrone compounds such as C-(phenyl)-N-(tert-butyl)nitrone (PBN) and derivatives thereof have been reported as possible therapeutics for the treatment of a wide variety of disease conditions arising from or characterized by oxidative damage or oxidative stress. Nitrone compounds exhibiting improved antioxidant activity compared to PBN can have better therapeutic potential than PBN. Aromatic nitrone breakdown, metabolism or degradation products such as N-alkyl hydroxylamines, N-alkyl hydronitroxides or nitric oxide may also contribute to the antioxidant properties of the aromatic nitrones, and contribute to their interruption of the inflammatory signaling pathways. Disease conditions arising from or characterized by oxidative damage or stress include, for example, disorders of the CNS and the PNS, such as stroke, Parkinson's disease, nerve damage and the like, and disorders of the peripheral organs, such as atherosclerosis, cardiac infarction, ulcerative colitis and the like.

A need exists for new classes of aromatic nitrone derivatives that have improved properties such as low toxicity, increased solubility, improved cellular and blood-brain-barrier permeability, and improved oral bioavailability.

### 3. SUMMARY OF THE INVENTION

Herein described are aromatic nitrone compounds that have improved antioxidant activity compared to PBN. The compounds of the invention are presented as potential therapeutic agents for indications that have been reported to be amenable to antioxidant treatment or that involve free-radical generation including, but not limited to: stroke, myocardial infarction and dysfunction, retinal ischemia and damage including macular degeneration and other degenerative disorders of the retina, renal ischemia, arteriosclerosis and other cardiovascular diseases, amyotrophic lateral sclerosis, Parkinson's disease, Alzheimer's disease, Huntington's disease, multiple sclerosis, head trauma and traumatic brain injury, nerve injury and neuropathies, migraine, schizophrenia and other disorders of cognition, mood disorders and other disorders of affect, pancreatitis and other pancreatic disorders, the treatment of diabetes and related complications, epilepsy, transplant and graft failure or rejection, hepatitis and jaundice-induced liver disorders, lung injury and damage, gastric ulcer, endotoxemia, aging and senescence, fetal damage due to intrauterine ischemia, the treatment and prophylaxis of pain syndromes (acute and chronic or neuropathic), arthritis and other autoimmune disorders, asthma and allergic reactions, inflammatory bowel disease, irritable bowel syndrome, uveitis, cancer, the treatment of complications and disorders arising from cancer therapy, and alopecia (hair loss).

The present invention provides aromatic nitrone compounds that are capable of modifying mammalian inflammatory pathways, pharmaceutical compositions having substituted aryl, heteroaromatic or bicyclic aryl nitrones as active ingredients and their use to treat, prevent or ameliorate a range of conditions in mammals such as, but not limited to, pain of various genesis or etiology, for example, acute, chronic, inflammatory and neuropathic pain, dental pain and headache (such as migraine, cluster headache and tension headache). The compounds of the present invention are also useful as anti-inflammatory agents for the treatment of arthritis, and as agents to treat Parkinson's disease, Alzheimer's disease, stroke, uveitis, asthma, myocardial infarction, traumatic brain injury, spinal cord injury, neurodegenerative disorders, alopecia (hair loss), inflammatory bowel disease, autoimmune disorders, renal disorders, obesity, eating disorders, cancer, schizophrenia, epilepsy, sleeping disorders, cognition, depression, anxiety, high blood pressure, lipid disorders and atherosclerosis.

In one aspect, the present invention provides aryl nitrone compounds that comprise a cycloalkenyl or aryl ring of 5 to 8 atoms. A first position of the ring is bonded to the carbon atom of a nitrone group. The carbon atom of the nitrone is further bonded to hydrogen, substituted or unsubstituted (C₁-C₆)alkyl, substituted or unsubstituted (C₁-C₆)cycloalkyl, substituted or unsubstituted aryl, or substituted or unsubstituted aralkyl. The nitrogen atom of the nitrone group is bonded to substituted or unsubstituted aliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, or substituted or unsubstituted heteroaralkyl. A second position of the ring, adjacent or ortho to the first position discussed above, is linked to a second group via either a direct bond or a methylene linker. The optional methylene linker can be substituted or unsubstituted. The second group is selected from sulfanyl, substituted sulfanyl, aminosulfonyl, substituted aminosulfonyl, sulfonic acid, sulfonic acid ester (i.e., sulfonate), carbamoyl, substituted carbamoyl, amino, substituted amino, hydroxyl, dihydroxyphosphoryl, substituted dihydroxyphosphoryl, aminohydroxyphosphoryl, substituted aminohydroxyphosphoryl, carboxy and substituted carboxy (i.e., ester). In some embodiments the ring is further substituted, while in other embodiments the ring is only substituted at the first and second positions. In preferred embodiments, the compounds of the invention do not encompass any of compounds 1-50 as described in Section 5.3.

In a particular embodiment of the invention, the ring of the compound is a phenyl ring. The phenyl ring can be substituted only with the first and second groups (at the first and second positions) discussed above, or the phenyl ring can be further substituted. When the phenyl ring is further substituted, in certain embodiments the phenyl ring is substituted with a third group at a position para to the position of the nitrone group. In particular embodiments, the substituents at the ortho and para positions, i.e. the second and third groups, are identical.

In a further aspect, the present invention provides pharmaceutical compositions comprising aryl nitrone compounds and a pharmaceutical carrier, excipient or diluent. In this aspect of the invention, the pharmaceutical composition can comprise an aryl nitrone compound described above. In addition, the pharmaceutical composition can also comprise one or more of compounds 1, 8, 9, 11, 16-22, 25-27, 37-43 and 45-50 described in Section 5.3.

In another aspect, the present invention provides heteroaromatic nitrone compounds that comprise a cycloheteroalkenyl or heteroaryl ring of 5 to 8 atoms. In particular embodiments of the invention, the ring of the compound is a furan, thiophene or pyrimidine ring. A first position of the ring is bonded to the carbon atom of a nitrone group. The carbon atom of the nitrone is further bonded to hydrogen, substituted or unsubstituted (C₁-C₆)alkyl, substituted or unsubstituted (C₁-C₆)cycloalkyl, substituted or unsubstituted aryl, or substituted or unsubstituted aralkyl. The nitrogen atom of the nitrone group is bonded to substituted or unsubstituted aliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, or substituted or unsubstituted heteroaralkyl. A second position of the ring, adjacent to the first position discussed above, is linked to a second group via either a direct bond or a methylene linker. The methylene linker can be substituted or unsubstituted. The second group is selected from sulfanyl, substituted sulfanyl, aminosulfonyl, substituted aminosulfonyl, sulfonic acid, sulfonic acid ester (i.e., sulfonate), carbamoyl, substituted carbamoyl, amino, substituted amino, hydroxyl, dihydroxyphosphoryl, substituted dihydroxyphosphoryl, aminohydroxyphosphoryl, substituted aminohydroxyphosphoryl, carboxy and substituted carboxy (i.e., ester). In some embodiments the ring is further substituted, while in other embodiments the ring is only substituted at the first and second positions. In preferred embodiments, the compounds of the invention do not encompass any of compounds 51-69 as described in Section 5.4.

The ring can be substituted only with the first and second groups (at the first and second positions) discussed above, or the ring can be further substituted. When the ring is further substituted, in certain embodiments the ring is substituted with a third group at a position two positions away from the position of the nitrone group in the opposite direction from the second group. For example, in a six-membered ring, the second group is adjacent to the nitrone and the third group is para to the second group. In particular embodiments, the second and third groups are identical.

In a further aspect, the present invention provides pharmaceutical compositions comprising heteroaromatic nitrone compounds and a pharmaceutical carrier, excipient or diluent. In this aspect of the invention, the pharmaceutical composition can comprise a heteroaromatic nitrone compound described above. In addition, the pharmaceutical composition can also comprise one or more of compounds 51 and 53-69 described in Section 5.4.

In yet another aspect, the present invention provides bicyclic aryl nitrone compounds that comprise a bicycloalkenyl, bicycloheteroalkenyl, bicycloaryl or bicycloheteroaryl ring of 8 to 11. A first position of the ring is bonded to the carbon atom of a nitrone group. The carbon atom of the nitrone is further bonded to hydrogen, substituted or unsubstituted (C₁-C₆)alkyl, substituted or unsubstituted (C₁-C₆)cycloalkyl, substituted or unsubstituted aryl, or substituted or unsubstituted aralkyl. The nitrogen atom of the nitrone group is bonded to substituted or unsubstituted aliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, or substituted or unsubstituted heteroaralkyl. A second position of the ring, adjacent to the first position discussed above, is linked to a second group via either a direct bond or a methylene linker. The methylene linker can be substituted or unsubstituted. The second group is selected from hydrogen, sulfanyl, substituted sulfanyl, aminosulfonyl, substituted aminosulfonyl, sulfonic acid, sulfonic acid ester (i.e., sulfonate), carbamoyl, substituted carbamoyl, amino, substituted amino, hydroxyl, dihydroxyphosphoryl, substituted dihydroxyphosphoryl, aminohydroxyphosphoryl, substituted aminohydroxyphosphoryl, carboxy and substituted carboxy (i.e., ester). In preferred embodiments, the compounds of the invention do not encompass any of compounds 70-78 as described in Section 5.5.

The ring can be substituted only with the first and second groups (at the first and second positions) discussed above, or the ring can be further substituted. When the ring is further substituted, in certain embodiments the ring is substituted with a third group at a position two positions away from the position of the nitrone group in the opposite direction from the second group. For example, in a six-membered ring, the second group is adjacent to the nitrone and the third group is para to the second group. In particular embodiments, the second and third groups are identical.

In a further aspect, the present invention provides pharmaceutical compositions comprising bicyclic aryl nitrone compounds and a pharmaceutical carrier, excipient or diluent. In this aspect of the invention, the pharmaceutical composition can comprise a bicyclic aryl nitrone compound described above. In addition, the pharmaceutical composition can also comprise one or more of compounds 70-78 described in Section 5.5.

In a method of treatment aspect, this invention provides a method of treating a mammal susceptible to or afflicted with a condition associated with arthritis, uveitis, asthma, myocardial infarction, traumatic brain injury, acute spinal cord injury, alopecia (hair loss), inflammatory bowel disease or autoimmune disorders, which method comprises administering an effective amount of one or more of the pharmaceutical compositions just described.

In yet another method of treatment aspect, this invention provides a method of treating a mammal susceptible to or afflicted with a condition that gives rise to pain responses or that relates to imbalances in the maintenance of basal activity of sensory nerves. Nitrone compounds have use as analgesics for the treatment of pain of various geneses or etiology, for example, acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain (such as that associated with gastroesophageal reflex disease, irritable bowel syndrome, inflammatory bowel disease, pancreatitis, and various gynecological and urological disorders); dental pain; and headache (such as migraine, cluster headache and tension headache).

In additional method of treatment aspects, this invention provides methods of treating a mammal susceptible to or afflicted with neurodegenerative diseases and disorders such as, for example, Parkinson's disease, Alzheimer's disease and multiple sclerosis; diseases and disorders which are mediated by or result in neuroinflammation such as, for example, traumatic brain injury, stroke and encephalitis; centrally-mediated neuropsychiatric diseases and disorders such as, for example, depression, mania, bipolar disease, anxiety and schizophrenia; eating disorders, sleep disorders and cognition disorders; epilepsy and seizure disorders; prostate, bladder and bowel dysfunction such as, for example, urinary incontinence, urinary hesitancy, rectal hypersensitivity, fecal incontinence, benign prostatic hypertrophy and inflammatory bowel disease; respiratory and airway diseases and disorders such as, for example, allergic rhinitis, asthma, reactive airway diseases and chronic obstructive pulmonary disease; diseases and disorders which are mediated by or result in inflammation such as, for example, rheumatoid arthritis, osteoarthritis, myocardial infarction, various autoimmune diseases and disorders, uveitis and atherosclerosis; itch/pruritus such as, for example, psoriasis; alopecia (hair loss); obesity; lipid disorders; cancer; high blood pressure; spinal cord injury; and renal disorders. The method comprises administering an effective condition-treating or condition-preventing amount of one or more of the pharmaceutical compositions just described.

In additional aspects, this invention provides methods for synthesizing the aryl, heteroaromatic and bicyclic aryl nitrone compounds of the invention.

### 4. BRIEF DESCRIPTION OF THE DRAWINGS

FIG. 1 is an illustration of representative oxidative synthetic pathways to aryl nitrone compounds of the invention.

FIG. 2 is an illustration of representative oxidative synthetic pathways to heteroaromatic nitrone compounds of the invention.

FIG. 3 is an illustration of representative oxidative synthetic pathways to bicyclic aryl nitrone compounds of the invention.

### 5. DETAILED DESCRIPTION OF THE INVENTION

5.1 Definitions

When describing compounds of the invention, pharmaceutical compositions containing such compounds and methods of using such compounds and compositions, the following terms have the following meanings unless otherwise indicated.

“Acyl” refers to a radical —C(O)R, where R is hydrogen, alkyl, cycloalkyl, cycloheteroalkyl, aryl, arylalkyl, heteroalkyl, heteroaryl or heteroarylalkyl as defined herein. Representative examples include, but are not limited to, formyl, acetyl, cylcohexylcarbonyl, cyclohexylmethylcarbonyl, benzoyl, benzylcarbonyl and the like.

“Acylamino” refers to a radical —NR′C(O)R, where R′ is hydrogen, alkyl, cycloalkyl, cycloheteroalkyl, aryl, arylalkyl, heteroalkyl, heteroaryl or heteroarylalkyl, and R is hydrogen, alkyl, alkoxy, cycloalkyl, cycloheteroalkyl, aryl, arylalkyl, heteroalkyl, heteroaryl or heteroarylalkyl, as defined herein. Representative examples include, but are not limited to, formylamino, acetylamino, cylcohexylcarbonylamino, cyclohexylmethylcarbonylamino, benzoylamino, benzylcarbonylamino and the like.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkenylene, alkynyl and alkynylene. Aliphatic groups typically have from 1 or 2 to about 12 carbon atoms.

“Alkyl” refers to monovalent saturated aliphatic hydrocarbyl groups preferably having from 1 to about 11 carbon atoms, more preferably from 1 to 8 carbon atoms, and still more preferably from 1 to 6 carbon atoms. The hydrocarbon chain may be either linear or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having from 1 to 6 carbon atoms. The term “alkyl” also includes “cycloalkyls” as defined below.

“Alkylene” refers to divalent saturated aliphatic hydrocarbyl groups preferably having from 1 to 11 carbon atoms and more preferably 1 to 6 carbon atoms which can be linear or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” refers to monovalent olefinically unsaturated hydrocarbyl groups preferably having from 2 to 11 carbon atoms and more preferably 2 to 6 carbon atoms which can be linear or branched and having at least 1 and preferably 1 to 2 sites of olefinic unsaturation. Preferred alkenyl groups include ethenyl (-CH=CH₂), n-propenyl (—CH₂CH═CH₂), isopropenyl (—C(CH₃)═CH₂) and the like.

“Alkenylene” refers to divalent olefinically unsaturated hydrocarbyl groups preferably having from 2 to 11 carbon atoms and more preferably 2 to 6 carbon atoms which can be linear or branched and having at least 1 and preferably 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically unsaturated hydrocarbyl groups preferably having from 2 to 11 carbon atoms and more preferably 2 to 6 carbon atoms which can be linear or branched and having at least 1 and preferably 1 to 2 sites of alkynyl unsaturation. Preferred alkynyl groups include ethynyl (—C≡CH), propargyl (—CH₂C≡CH) and the like.

“Alkoxy” refers to the group “—O-alkyl” where alkyl is as defined above. Preferred alkoxy groups include, by way of example, methoxy, ethoxy, n-propoxy, isopropoxy, n-butoxy, tert-butoxy, sec-butoxy, n-pentyloxy, n-hexyloxy, 1,2-dimethylbutoxy and the like.

“Alkanoyl” as used herein refers to the group —C(O)—R, where R is alkyl as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. An aryl group preferably comprises from 6 to 14 carbon atoms.

“Fused Aryl” refers to an aryl having two of its ring carbon atoms in common with a second aryl ring or with an aliphatic ring.

“Alkaryl” refers to an aryl group, as defined above, substituted with one or more alkyl groups, as defined above.

“Aralkyl” or “arylalkyl” refers to an alkyl group, as defined above, substituted with one or more aryl groups, as defined above.

“Aryloxy” refers to —O-aryl groups wherein “aryl” is as defined above.

“Alkylamino” refers to the group —NRR′, wherein at least one of R and R′ is alkyl as defined above.

“Arylamino” refers to the group —NRR′, wherein at least one of R and R′ is aryl as defined above.

“Alkoxyamino” refers to a radical —N(R)OR′ where R may be hydrogen or alkyl and R′ represents an alkyl or cycloalkyl group as defined herein.

“Alkoxycarbonyl” refers to a radical —C(O)-alkoxy where alkoxy is as defined herein.

“Alkylarylamino” refers to a radical —NRR′ where R represents an alkyl or cycloalkyl group and R′ is an aryl group as defined herein.

“Alkylsulfonyl” refers to a radical —S(O)₂R where R is an alkyl or cycloalkyl group as defined herein. Representative examples include, but are not limited to, methylsulfonyl, ethylsulfonyl, propylsulfonyl, butylsulfonyl and the like.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR where R is an alkyl or cycloalkyl group as defined herein that may be optionally substituted as defined herein. Representative examples include, but are not limited to, methylthio, ethylthio, propylthio, butylthio and the like.

“Amino” refers to the radical —NH₂.

“Arylalkyloxy” refers to an —O-arylalkyl radical where arylalkyl is as defined herein.

“Aryloxycarbonyl” refers to a radical —C(O)—O-aryl where aryl is as defined herein.

“Arylsulfonyl” refers to a radical —S(O)₂R where R is an aryl group as defined herein.

“Azido” refers to the radical —N₃.

“Carbamoyl” refers to the radical —C(O)N(R)₂ where each R group is independently hydrogen, alkyl, cycloalkyl or aryl, as defined herein, which may optionally be substituted as defined herein.

“Carboxy” refers to the radical —C(O)OH.

“Carboxyamino” refers to the radical —N(R)C(O)OH where R may be hydrogen or alkyl as defined herein.

“Cycloalkyl” refers to cyclic hydrocarbyl groups having from 3 to 10 carbon atoms and having a single cyclic ring or multiple condensed rings, including fused and bridged ring systems, which optionally can be substituted with 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl and the like, and multiple ring structures such as adamantanyl and the like.

“Cycloalkenyl” refers to cyclic hydrocarbyl groups having from 3 to 10 carbon atoms, a single cyclic ring or multiple condensed rings, including fused and bridged ring systems, and at least 1 and preferably 1 to 2 sites of olefinic unsaturation. Such cycloalkenyl groups include, by way of example, single ring structures such as cyclohexenyl, cyclopentenyl, cyclopropenyl and the like.

“Fused cycloalkenyl” refers to a cycloalkenyl group having two of its ring carbon atoms in common with a second aliphatic or aromatic ring. The location of its olefinic unsaturation may impart aromaticity to the cycloalkenyl ring.

“Cyanato” refers to the radical —OCN.

“Cyano” refers to the radical —CN.

“Dialkylamino” means a radical —NRR′ where R and R′ each independently represent an alkyl, substituted alkyl, aryl, substituted aryl, cycloalkyl, substituted cycloalkyl, cycloheteroalkyl, substituted cycloheteroalkyl, heteroaryl or substituted heteroaryl group as defined herein.

“Halo” or “halogen” refers to fluoro, chloro, bromo and iodo. Preferred halo groups are either fluoro or chloro.

“Hydroxy” refers to the radical —OH.

“Nitro” refers to the radical —NO₂.

“Substituted” refers to a group in which one or more hydrogen atoms are each independently replaced with the same or different substituent(s). Typical substituents include, but are not limited to, —X, —R¹⁴, —OH, ═O, —OR¹⁴, —SR¹⁴, —SH, ═S, —NR¹⁴R¹⁵, ═NR¹⁴, —CX₃, —CF₃, —CN, —OCN, —SCN, —NO, —NO₂, ═N₂, —N₃, —S(O)₂OH, —S(O)₂OR¹⁴, —S(O)₂R¹⁴, —OS(O₂)OH, —OS(O)₂OR¹⁴, —P(O)(OH)₂, —P(O)(OR¹⁴)(OH), —OP(O)(OR¹⁴)(OR¹⁵), —C(O)R¹⁴, —C(S)R¹⁴, —C(O)OR¹⁴, —C(O)NR¹⁴R¹⁵, —C(O)OH, —C(S)OR¹⁴, —NR¹⁶C(O)NR¹⁴R¹⁵, —NR¹⁶C(S)NR¹⁴R¹⁵, —NR¹⁷C(NR¹⁶)NR¹⁴R¹⁵ and —C(NR¹⁶)NR¹⁴R¹⁵, where each X is independently a halogen; R¹⁴, R¹⁵, R¹⁶ and R¹⁷ are each independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR¹⁸R¹⁹, —C(O)R¹⁸ or —S(O)₂R¹⁸, where R¹⁸ and R¹⁹ together with the atom to which they are both attached may optionally form a cycloheteroalkyl or substituted cycloheteroalkyl ring; and R¹⁸ and R¹⁹ are each independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following: 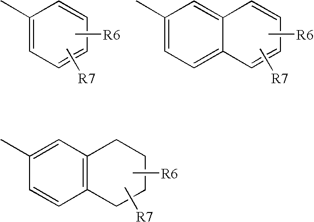
In these formulas one of R⁶ and R⁷ may be hydrogen and at least one of R⁶ and R⁷ is independently selected from alkyl, alkenyl, alkynyl, cycloheteroalkyl, acyl, alkoxy, aryloxy, heteroaryloxy, alkylamino, arylamino, heteroarylamino, —NR¹⁰C(O)R¹¹, —NR¹⁰S(O)R¹¹, —NR¹⁰SO₂R¹¹, —C(O)Oalkyl, —C(O)Oaryl, —C(O)NR¹⁰R¹¹, —C(O)NR¹⁰OR¹¹, —NR¹⁰R¹¹, —SO₂NR¹⁰R¹¹, —S-alkyl, —S(O)alkyl, —SO₂alkyl, —S-aryl, —S(O)aryl and SO₂aryl, or R⁶ and R⁷ may be joined to form a cyclic ring (saturated or unsaturated) from 5 to 8 atoms, optionally containing one or more heteroatoms selected from the group N, O or S. R¹⁰ and R¹¹ are each independently hydrogen, alkyl, alkenyl, alkynyl, perfluoroalkyl, cycloalkyl, cycloheteroalkyl, aryl, substituted aryl, heteroaryl, substituted heteroaryl or the like.

“Hetero” when used to describe a compound or a group present on a compound means that one or more carbon atoms in the compound or group have been replaced by a nitrogen, oxygen or sulfur heteroatom. Hetero may be applied to any of the hydrocarbyl groups described above such as alkyl (i.e., heteroalkyl), cycloalkyl (i.e., cycloheteroalkyl), aryl (i.e., heteroaryl), cycloalkenyl (i.e., cycloheteroalkenyl) and the like, such groups having from 1 to 5 and preferably from 1 to 3 heteroatoms.

“Heteroaryl” or “heteroaromatic” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, P-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like. The heteroaryl group is preferably a 5- to 20-membered heteroaryl group and more preferably a 5- to 10-membered heteroaryl group. Preferred heteroaryl groups are those derived from thiophene, pyrrole, benzothiophene, benzofuran, indole, pyridine, quinoline, imidazole, oxazole and pyrazine.

Examples of representative heteroaryls include the following: 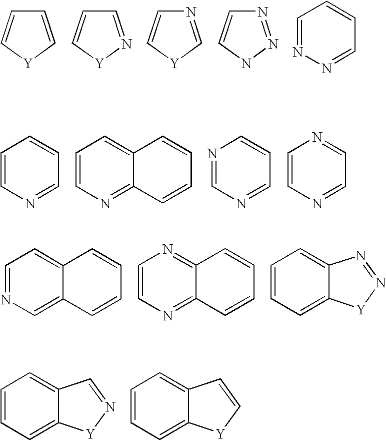
wherein Y is selected from NH, NR⁴, O, and S.

Examples of representative cycloheteroalkyls include the following: 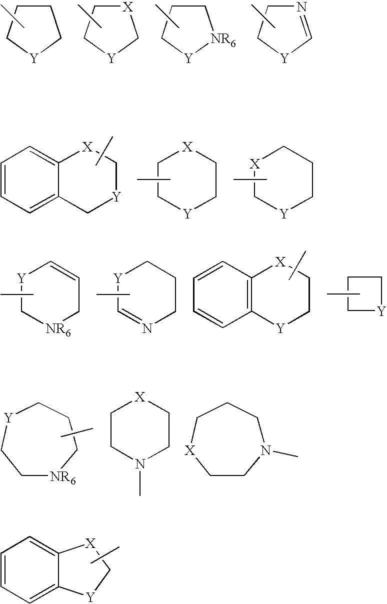
wherein X is selected from CR³R⁴, NR⁴, O and S, and Y is selected from NH, NR⁴, O and S.

Examples of representative cycloheteroalkenyls include the following: 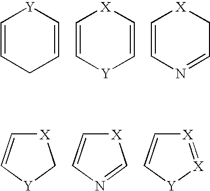
wherein each X is selected from CR³R⁴, CR⁴, N, NR⁴, O and S, and Y is selected from NH, NR⁴, O and S.

Examples of representative aryls containing heteroatoms and substitution include the following: 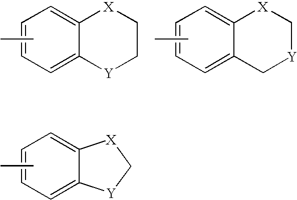
wherein X is selected from CR³R⁴, NR⁴, O and S, and Y is selected from NH, NR⁴, O and S.

“Hetero substituent” refers to a halo, O, S or N atom-containing functionality that may be present as R⁴ in a C—R⁴ group which is in turn present as a substituent directly on A, Q, W, X, Y or Z of the compounds of this invention. Such a functionality may also be present as a substituent in “substituted” aryl and aliphatic groups present in the compounds.

Examples of hetero substituents include:
-halo, —CN;
—OH, —OR, —SR;
—NO₂, —NH₂, —NHR, —NRR′;
—NRC(O)R′, —NRS(O)R′, —NRSO₂R′;
—CO₂H, —CO₂R, —C(O)NRR′, —C(O)N(R)OR′; and
—SO₃H, —SO₃R, —SO₂R, —S(O)R and —SO₂NRR′,
   wherein R and R′ are each independently aryl or aliphatic and optionally contain substitution. Among hetero substituents containing R and/or R′ groups, preference is given to those substituents having an aryl or alkyl R or R′ group as defined herein. Preferred hetero substituents are those listed above.

One of ordinary skill in the art of organic synthesis will recognize that the maximum number of heteroatoms in a stable, chemically feasible heterocyclic ring, whether aromatic or non-aromatic, is determined, inter alia, by the size of the ring, the degree of unsaturation of the ring and the valence of the heteroatoms. In general, a heterocyclic ring may have 1 to 4 heteroatoms so long as the heteroaromatic ring is chemically feasible and stable.

“Pharmaceutically acceptable” means approved by a regulatory agency of the federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include:
(1) acid addition salts formed with
   (a) inorganic acids such as hydrochloric acid, hydrobromic acid, sulfuric acid, nitric acid, phosphoric acid and the like, or
   (b) organic acids such as acetic acid, propionic acid, hexanoic acid, cyclopentylpropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid, muconic acid and the like; or
(2) salts formed when an acidic proton present in the parent compound either
   (a) is replaced by a metal ion, e.g., an alkali metal ion, an alkaline earth ion or an aluminum ion, or
   (b) coordinates with an organic base such as ethanolamine, diethanolamine, triethanolamine, N-methylglucamine and the like.

Salts further include, by way of example only, sodium, potassium, calcium, magnesium, ammonium, tetraalkylammonium and the like, and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to a non-toxic, pharmaceutically acceptable cationic counterion of an acidic functional group. Such cations are exemplified by sodium, potassium, calcium, magnesium, ammonium and tetraalkylammonium cations and the like.

“Dihydroxyphosphoryl” or “phosphono” refers to the radical —PO(OH)₂.

“Substituted dihydroxyphosphoryl” refers to a dihydroxyphosphoryl radical wherein one or both of the hydroxyl groups are substituted. Suitable substituents are described in detail above.

“Aminohydroxyphosphoryl” refers to the radical —PO(OH)NH₂.

“Substituted aminohydroxyphosphoryl” refers to an aminohydroxyphosphoryl group wherein the amino group is substituted with one or two substituents. Suitable substituents are described in detail above. In certain embodiments, the hydroxyl group can also be substituted.

“Pharmaceutically acceptable vehicle” refers to a diluent, adjuvant, excipient or carrier with which a compound of the invention is administered.

“Preventing” or “prevention” refers to a reduction in the risk of acquiring a disease or disorder (i.e., causing at least one of the clinical symptoms of the disease not to develop in a subject that may be exposed to or predisposed to the disease but does not yet experience or display symptoms of the disease).

“Subject” includes humans. The terms “human,” “patient” and “subject” are used interchangeably herein.

“Sulfanyl” or “thiol” refers to the radical —SH. “Substituted sulfanyl” or “thio ether” refers to a radical such as —SR wherein R is any substituent described herein.

“Sulfonyl” refers to the divalent radical —S(O)₂—. “Substituted sulfonyl” refers to a radical such as —S(O)₂R wherein R is any substituent described herein. “Aminosulfonyl” or “sulfoamido” refers to the radical —S(O)2NH₂, and “substituted aminosulfonyl” or “substituted sulfoamido” refers to a radical such as —S(O)₂NR₂ wherein each R is independently any substituent described herein.

“Therapeutically effective amount” means an amount of a compound that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

“Prodrugs” are derivatives of the compounds of the invention which have metabolically cleavable groups and become by solvolysis or under physiological conditions the compounds of the invention which are pharmaceutically active in vivo. Such examples include, but are not limited to, choline ester derivatives and the like and N-alkylmorpholine esters and the like.

Other derivatives of the compounds of this invention have activity in both their acid and acid-derivative forms. An acid-sensitive form often offers advantages of solubility, tissue compatibility or delayed release in the mammalian organism (See H. Bundgard,1985, Design of Prodrugs, Elsevier, Amsterdam, pp. 7-9, 21-24). Prodrugs include acid derivatives well known to practitioners of the art, such as, for example, esters prepared by reaction of the parent acid with a suitable alcohol, amides prepared by reaction of the parent acid compound with a substituted or unsubstituted amine, acid anhydrides and mixed anhydrides. Simple aliphatic or aromatic esters, amides and anhydrides derived from acidic groups pendant on the compounds of this invention are preferred prodrugs. In some cases it is desirable to prepare double ester-type prodrugs such as (acyloxy)alkyl esters or ((alkoxycarbonyl)oxy)alkyl esters. Preferred are C1-C8 alkyl, C2-C8 alkenyl, aryl, C7-C12 substituted aryl and C7-C12 arylalkyl esters of the compounds of the invention.

It is to be understood that compounds having the same molecular formula but differing in the nature or sequence of bonding of their atoms or in the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is designated (R) or (S) according to the rules of Cahn and Prelog, or can be characterized by the manner in which the molecule rotates the plane of polarized light and is designated dextrorotatory or levorotatory (i.e., as (+)- or (−)-isomers, respectively). A chiral compound can exist as either individual enantiomer or as a mixture thereof. A mixture containing equal proportions of enantiomers is called a “racemic mixture”.

The compounds of this invention may possess one or more asymmetric centers; such compounds can therefore be produced as the individual (R)- or (S)-enantiomer or as a mixture thereof. Unless indicated otherwise, the description or naming of a particular compound in the specification and claims is intended to include both individual enantiomers and mixtures, racemic or otherwise, thereof. Methods for determination of stereochemistry and separation of stereoisomers are well-known in the art.

5.2 Aryl, Heteroaromatic and Bicyclic Aryl Nitrone Compounds

The present invention provides aryl, heteroaromatic and bicyclic aryl nitrone compounds useful for preventing and/or treating arthritis, Parkinson's disease, Alzheimer's disease, stroke, uveitis, asthma, myocardial infarction, pain syndromes (acute and chronic or neuropathic), traumatic brain injury, acute spinal cord injury, neurodegenerative disorders, alopecia (hair loss), inflammatory bowel disease and autoimmune disorders or conditions in mammals.

In certain embodiments, the present invention provides aryl, heteroaromatic and bicyclic aryl nitrone compounds according to formula (I): 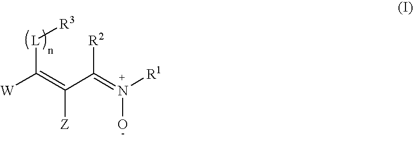
wherein:
for aryl nitrones, W and Z are joined to form a cycloalkenyl or aryl ring of 5 to 8 atoms, and said ring is substituted only with the -(L)ₙ-R³ and —C(R²)═N(O)—R¹ moieties of formula (I) or said ring is further substituted;
for heteroaromatic nitrones, W and Z are joined to form a cycloheteroalkenyl or heteroaryl ring of 5 to 8 atoms, and said ring is substituted only with the -(L)ₙ-R³ and —C(R²)═N(O)—R¹ moieties of formula (I) or said ring is further substituted;
for bicyclic aryl nitrones, W and Z are joined to form a bicycloalkenyl, bicycloheteroalkenyl, bicycloaryl, or bicycloheteroaryl ring of 8 to 11 atoms and said ring is substituted only with the -(L)ₙ-R³ and —C(R²)═N(O)—R¹ moieties of formula (I) or said ring is further substituted;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
for aryl nitrones, R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
for heteroaromatic and bicyclic aryl nitrones, R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂, and —CO₂R⁹;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
n is an integer from 0 to 1; or
a pharmaceutically acceptable salt or prodrug thereof.

In other embodiments of heteroaromatic and bicyclic aryl nitrones of formula (I), R³ is selected from —SR⁹, —SO₂NR⁷ R⁸, —SO₃R⁹, —CONR⁷R⁸, —NR⁷R⁸, —OH, —PO(OR⁹)NR⁷R⁸, —PO(N⁷R⁸)₂, —PO(OR⁹)₂ and —CO₂R⁹.

Among the aryl nitrones described above by formula (I), there is a general preference for compounds wherein W and Z are joined to form a 6-membered aryl ring.

Among the heteroaromatic nitrones described above by formula (I), there is a general preference for compounds wherein W and Z are joined to form a 6-membered heteroaryl ring. However, the heteroaryl ring can be any 5- to 8-membered heteroaryl ring known to those of skill in the art, including the exemplary heteroaryl rings described in the Definitions section (Section 5.1) above. In certain embodiments, the heteroaryl ring is a pyridine, pyrimidine, furan, thiophene or pyrrole ring.

Referring to bicyclic aryl nitrones of formula (I), in certain embodiments R¹ is substituted with a group other than phenyl, substituted phenyl or methyl. In other embodiments R¹ is substituted with a group other than phenyl, substituted phenyl or lower alkyl. For instance, R¹ can be substituted or unsubstituted heteroalkyl, substituted or unsubstituted acyl, substituted or unsubstituted heteroaryl, substituted or unsubstituted aralkyl, or substituted or unsubstituted heteroaralkyl.

Also referring to bicyclic aryl nitrones of formula (I), in certain embodiments R² can be substituted with a group other than hydrogen. For instance, R² can be substituted or unsubstituted (C₁-C₆)alkyl, substituted or unsubstituted (C₁-C₆)cycloalkyl, substituted or unsubstituted aryl, or substituted or unsubstituted aralkyl.

In further embodiments of bicyclic aryl nitrones of formula (I), R³ can be substituted with a group other than —OH, —SMe or —S(C₆H₅). For instance, R³ can be selected from —SO₂NR⁷R⁸, —SO₃R⁹, —CONR⁷R⁸, —NR⁷R⁸, —PO(OR⁹)NR⁷R⁸, —PO(NR⁷R⁸)₂, —PO(OR⁹)₂ and —CO₂R⁹.

Referring again to bicyclic aryl nitrones of formula (1), in certain embodiments W and Z are joined to form a six-membered ring that is fused to a second ring. The second ring can be, for instance, a five- or six-membered ring and can contain heteroatom(s). The second ring can be fused to any adjacent pair of atoms in the first ring.

Also referring to bicyclic aryl nitrones of formula (I), in certain embodiments W and Z are joined to form a seven-membered ring that is fused to a second ring. The second ring can be, for instance, a five-membered ring and can contain heteroatom(s). The second ring can be fused to any adjacent pair of atoms in the first ring. For example, the bicyclic aromatic ring can be azulene.

In further embodiments, the present invention provides aryl and heteroaromatic nitrone compounds according to formula (II): 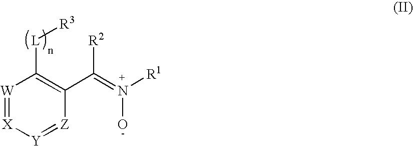
wherein:
for aryl nitrones, each of W, X, Y and Z is independently C—R⁴;
for heteroaromatic nitrones, m of W, X, Y and Z is N and the remainder are each independently C—R⁴;
L is C(R ²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
for aryl nitrones, R³ is selected from —SR⁹, —SO₂NR⁷R⁸, —SO₃R⁹, —CONR⁷R⁸, —NR⁷R⁸, —OH, —PO(OR⁹)NR⁷R⁸, —POR⁷R⁸)₂, —PO(OR⁹)₂ and —CO₂R⁹;
for heteroaromatic nitrones, R³ is selected from hydrogen, —SR⁹, —SO₂NR⁷R⁸, —SO₃R⁹, —CONR⁷R, —NR⁷R⁸, —OH, —PO(OR⁹)NR⁷R⁸, —PO(NR⁷R⁸)₂, —PO(OR⁹)₂ and —CO₂R⁹
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl and, substituted or unsubstituted heteroaralkyl;
n is an integer from 0 to 1; and
for heteroaromatic nitrones, m is an integer from 1 to 3; or
a pharmaceutically acceptable salt or prodrug thereof.

In other embodiments of heteroaromatic nitrones of formula (II), R³ is selected from —SR⁹, —SO₂NR⁷R⁸, —SO₃R⁹, —CONR⁷R⁸, —NR⁷R, -OH, —PO(OR⁹)NR⁷R⁸, —PO(NR⁷R⁸)₂, —PO(OR⁹)₂ and —CO₂R⁹.

In certain embodiments of heteroaromatic nitrones of formula (II), X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. While the substituents at R³ and R⁵ can vary independently, in certain embodiments R³ and R⁵ are identical. In particular embodiments, n is 0 and R³ and R⁵ are identical.

5.3 Aryl Nitrone Compounds

In additional embodiments, the present invention provides aryl nitrone compounds according to formula (III): 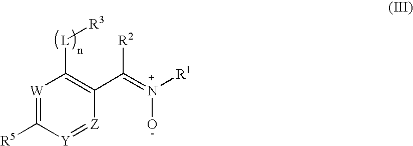
wherein:
each of W, Y and Z is independently C—R⁴;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R ⁵is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
n is an integer from 0 to 1; or
a pharmaceutically acceptable salt or prodrug thereof.

While the substituents at R³ and R⁵ can vary independently, in certain embodiments R³ and R⁵ are identical. In particular embodiments, n is 0 and R³ and R⁵ are identical.

In preferred embodiments, the aryl nitrone compounds according to formula (I), formula (II) or formula (III) do not encompass any of compounds 1-50. (Compounds 1-78 in Sections 5.3-5.5 are distinct from the compounds in Examples 1-92.) In particular embodiments, the aryl nitrone compounds according to formula (I), formula (II) or formula (III) do not encompass any salt of compounds 1-50. In further embodiments, the aryl nitrone compounds according to formula (I), formula (II) or formula (III) do not encompass any isomer, diastereomer or enantiomer of compounds 1-50. Compounds 1-50 follow:
1. 1,3-Benzenedisulfonic acid, 4-[[(1-methylethyl)oxidoimino]methyl]-, disodium salt {or N-isopropyl-C-(2,4-disulfophenyl)nitrone, disodium salt};
2. 1,3-Benzenedisulfonic acid, 4-[[(1,1-dimethylethyl)oxidoimino]methyl]-{or N-(tert-butyl)-C-(2,4-disulfophenyl)nitrone};
3. 1,3-Benzenedisulfonic acid, 4-[[(2-hydroxy-1,1-dimethylethyl)oxidoimino]methyl]-, disodium salt {or N-(2-hydroxy-1,1-dimethylethyl)-C-(2,4-disulfophenyl)nitrone, disodium salt};
4. 1,3-Benzenedisulfonic acid, 4-[[(1,1-dimethylethyl)oxidoimino]methyl]-, disodium salt for N-(tert-butyl)-C-(2,4-disulfophenyl)nitrone, disodium salt};
5. Benzenesulfonic acid, 2-[[(1,1-dimethylethyl)oxidoimino]methyl]-, ion(1-) (or N-(tert-butyl)-C-(2-sulfophenyl)nitrone, ion(-1)};
6. 1,3-Benzenedisulfonic acid, 4-[[(1,1-dimethylethyl)oxidoimino]methyl]-, diammonium salt (or N-(tert-butyl)-C-(2,4-disulfophenyl)nitrone, diammonium salt};
7. 1,3-Benzenedisulfonic acid, 4-[[(1,1-dimethylethyl)oxidoimino]methyl]-, magnesium salt (1:1) {or N-(tert-butyl)-C-(2,4-disulfophenyl)nitrone, magnesium salt (1:1)};
8. 1,3-Benzenedisulfonic acid, 4-[(ethyloxidoimino)methyl]-, disodium salt for N-ethyl-C-(2,4-disulfophenyl)nitrone, disodium salt};
9. 1,3-Benzenedisulfonic acid, 4-[(butyloxidoimino)methyl]-, disodium salt (or N-butyl-C-(2,4-disulfophenyl)nitrone, disodium salt};
10. 1,3-Benzenedisulfonic acid, 4-[[(1,1-dimethylethyl)oxidoimino]methyl]-, ion(2-) {or N-(tert-butyl)-C-(2,4-disulfophenyl)nitrone, ion(-2)};
11. 1,3-Benzenedisulfonic acid, 4-[[(1,1-dimethylpropyl)oxidoimino]methyl]-, disodium salt {or N-(1,1-dimethylpropyl)-C-(2,4-disulfophenyl)nitrone, disodium salt};
12. 1,3-Benzenedisulfonic acid, 4-[[(1,1-dimethylethyl)oxidoimino]methyl]-, dipotassium salt {or N-(tert-butyl)-C-(2,4-disulfophenyl)nitrone, dipotassium salt};
13. Benzenesulfonic acid, 2-[[(1,1-dimethylethyl)oxidoimino]methyl]-{or N-(tert-butyl)-C-(2-sulfophenyl)nitrone};
14. Benzenesulfonic acid, 2-[[(1,1-dimethylethyl)oxidoimino]methyl]-, sodium salt {or N-(tert-butyl)-C-(2-sulfophenyl)nitrone, sodium salt};
15. 1,3-Benzenedisulfonic acid, 4-[[(1,1-dimethylethyl)oxidoimino]methyl]-, calcium salt (1:1) {or N-(tert-butyl)-C-(2,4-disulfophenyl)nitrone, calcium salt (1:1)};
16. Benzenesulfonic acid, 2-[[oxido(pentamethylphenyl)imino]methyl]-, sodium salt {or N-(pentamethylphenyl)-C-(2-sulfophenyl)nitrone, sodium salt};
17. Benzeneethanamine, N-[[2-(phenylthio)phenyl]methylene]-, N-oxide, (E)-{or (E)-N-(2-phenylethyl)-C-[2-(phenylthio)phenyl]nitrone};
18. Cyclohexanamine, 2-nitro-N-[[2-(phenylthio)phenyl]methylene]-, N-oxide {or N-(2-nitrocyclohexyl)-C-[2-(phenylthio)phenyl]nitrone};
19. Benzenamine, 4-(phenylthio)-N-[[2-(phenylthio)phenyl]methylene]-, N-oxide, (Z)-; {or (Z)-N-[4-(phenylthio)phenyl]-C-[2-(phenylthio)phenyl]nitrone};
20. Benzeneethanamine, N-[[2-(phenylthio)phenyl]methylene]-, N-oxide, (Z)-{or (Z)-N-(2-phenylethyl)-C-[2-(phenylthio)phenyl]nitrone};
21. Nitrone, α-carboxy-N-methyl-α-[2-(methylthio)phenyl]-, methyl ester {or N-methyl-C-[2-(methylthio)phenyl]-C′-(methoxcarbonyl)nitrone};
22. Glycine, N-[(2-methoxyphenyl)methylene]-, anhydride with diphenylborinic acid, N-oxide {or N-[2-(diphenylboron)oxy-2-oxoethyl]-C-(2-methoxyphenyl)nitrone};
23. 2-Propanamine, N-[(2-ethoxyphenyl)methylene]-2-methyl-, N-oxide {or N-(tert-butyl)-C-(2-ethoxyphenyl)nitrone};
24. Phenol, 2-[[(1,1-dimethylethyl)oxidoimino]methyl]-6-ethoxy-{or N-(tert-butyl)-C-(3-ethoxy-2-hydroxyphenyl)nitrone};
25. 2-Thiazolidinethione, 4-[[(2-methoxyphenyl)methylene]oxidoamino]-3,5,5-trimethyl-{or N-(3,5,5-trimethyl-2-thioxothiazolidin-4-yl)-C-(2-methoxyphenyl)nitrone};
26. Acetic acid, [2-[[(4-chlorophenyl)oxidoimino]methyl]phenoxy]-{or N-(4-chlorophenyl)-C-[2-(carboxymethoxy)phenyl]nitrone};
27. Benzenamine, N-[[2-(2-furanylmethoxy)phenyl]methylene]-, N-oxide {or N-phenyl-C-[2-(2-furanylmethoxy)phenyl]nitrone};
28. Nitrone, α-(2,3-dihydroxyphenyl)-N-phenyl-{or N-phenyl-C-(2,3-dihydroxyphenyl)nitrone};
29. Phenol, 2-[(oxidophenylimino)methyl]-, radical ion(1-) {or N-phenyl-C-(2-hydroxyphenyl)nitrone, radical ion(−1)};
30. Phenol, 2-[[oxido[2-(phenylthio)phenyl]imino]methyl]-{or N-[2-(phenylthio)phenyl]-C-(2-hydroxyphenyl)nitrone};
31. Phenol, 2-[[oxido[2-(phenylthio)phenyl]imino]methyl]-;
32. Nitrone, α-(2,3 -dihydroxyphenyl)-N-phenyl-;
33. Phenol, 2-[[[(2-hydroxyphenyl)methylene]oxidoamino]methyl]-{or N-[(2-hydroxyphenyl)methyl]-C-(2-hydroxyphenyl)nitrone};
34. Phenol, 2-[[(1,1-dimethylethyl)oxidoimino]methyl]-6-fluoro-{or N-(tert-butyl)-C-(3-fluoro-2-hydroxyphenyl)nitrone};
35. Phenol, 2-[(oxidophenylimino)methyl]-, radical ion(1-);
36. Phenol, 2-[[(1,1-dimethylethyl)oxidoimino]methyl]-6-ethoxy-{or N-(tert-butyl)-C-(3-ethoxy-2-hydroxyphenyl)nitrone};
37. Acetamide, N-[2-[[(1,1-dimethylethyl)oxidoimino]methyl]phenyl]-{or N-(tert-butyl)-C-[2-(acetamido)phenyl]nitrone};
38. 2-Butenamide, 4,4,4-trifluoro-N-[2-[[oxido(phenylmethyl)imino]methyl]phenyl]-3-(trifluoromethyl)-(or N-benzyl-C-{2-[3,3-bis(trifluoromethyl)-2-propenamido]phenyl}nitrone);
39. Glycine, N-(2-acetamido-5-chloro-a-phenylbenzylidene)-, methyl ester, N-oxide {or N-(2-methoxy-2-oxoethyl)-C-[2-(acetamido)-5-chlorophenyl]-C′-phenylnitrone};
40. 2-Butenamide, 4,4,4-trifluoro-3-methyl-N-[2-[[oxido(phenylmethyl)imino]methyl]phenyl]-(or N-benzyl-C-{2-[3-methyl-3-(trifluoromethyl)-2-propenamido]phenyl}nitrone);
41. Benzamide, N-[5-chloro-2-[[[4-(dimethylamino)phenyl]oxidoimino]methyl]phenyl]-{or N-[4-(NN-dimethylamino)phenyl]-C-[2-(benzamido)-4-chlorophenyl]nitrone};
42. Nitrone, α-(2-acetamido-5-chlorophenyl)-N-(carboxymethyl)-α-Phenyl {or N-(carboxymethyl)-C-[2-(acetamido)-5-chlorophenyl]-C′-phenylnitrone};
43. Nitrone, N-(carboxymethyl)-α-[5-chloro-2-(methylamino)phenyl]-α-phenyl {or N-(carboxymethyl)-C-[2-(methylamino)-5-chlorophenyl]-C′-phenylnitrone};
44. Benzoic acid, 2-[[(1,1-dimethylethyl)oxidoimino]methyl]-{or N-(tert-butyl)-C-(2-carboxyphenyl)nitrone};
45. Nitrone, α-(o-carboxyphenyl)-N-3,4-xylyl-{or N-(3,4-dimethylphenyl)-C-(2-carboxyphenyl)nitrone};
46. o-Veratric acid, 6-(N-methylformimidoyl)-, N-oxide, sodium salt {or N-methyl-C-(2-carboxy-3,4-dimethoxyphenyl)nitrone, sodium salt};
47. o-Veratric acid, 6-(N-methylformimidoyl)-, N-oxide, methyl ester {or N-methyl-C-[2-(methoxycarbonyl)-3,4-dimethoxyphenyl]nitrone};
48. o-Veratric acid, 6-(N-methylformimidoyl)-, N-oxide {or N-methyl-C-(2-carboxy-3,4-dimethoxyphenyl)nitrone};
49. Benzoic acid, 2-[(oxidophenylimino)methyl]-, methyl ester {or N-phenyl-C-[2-(methoxycarbonyl)phenyl]nitrone}; and
50. Benzoic acid, 2-[(oxidophenylimino)methyl-, N-oxide {or N-phenyl-C-(2-carboxyphenyl)nitrone}.

In the aryl nitrone compounds according to formulas (I)-(III), the rings can be substituted only with the groups depicted in the formulas, or they can be further substituted. For instance, in formula (I), the ring comprising W and Z can be substituted only with the depicted -(L)ₙ-R³ and —C(R²)═N(O)—R¹ moities. Alternatively, the ring comprising W and Z can comprise further substituents at any position in the ring. In formulas (II) and (III), the six-membered ring can comprise the two or three substituents depicted, respectively, or the ring can comprise further substituents.

In certain embodiments of aryl nitrone compounds of formulas (I)-(III), R is a substituent comprising a sulfur or phosphorus atom. For instance, R³ can be selected from —SO₂NR⁷R⁸, —SO₃R⁹, —PO(OR⁹)NR⁷R⁸, —PO(N⁷R⁸)₂ and —PO(OR⁹)₂.

In some embodiments, R³ is —SO₃H and R¹ is not lower alkyl, acetylated lower alkyl, hydroxylated lower alkyl, phenyl or substituted phenyl. In further embodiments, R³ is —SR⁹ and R⁹ is other than phenyl or methyl.

In additional embodiments, R³ is —SR⁹ and R¹ is other than —CH₂(C₆H₅), substituted —CH₂(C₆H₅), cyclohexyl, substituted cyclohexyl, methyl, phenyl or substituted phenyl. In further embodiments, R³ is —SR⁹ and R⁹ is other than phenyl or methyl.

In other embodiments, R³ is a hydroxyl group. For instance, R³ can be —OH when R¹ is other than —CH₂(C₆H₅), substituted —CH₂(C₆H₅), lower alkyl, phenyl or substituted phenyl.

In further embodiments, R³ can comprise a nitrogen atom. For instance, R³ can be an amino group. In certain embodiments, R³ can comprise a nitrogen atom where R³ is —NR⁷R⁸ and R⁷ and R⁸ are both other than hydrogen. In further embodiments, R³ can comprise a nitrogen atom where R³is —NR⁷R⁸ and R¹ is other than —CH₂CO₂Me, —CH₂CO₂H, —CH₂(C₆H₅), substituted —CH₂(C₆H₅), lower alkyl, phenyl or substituted phenyl.

In addition, in certain embodiments R³ is a carboxy or substituted carboxy (i.e., ester) group. In particular embodiments, R³ is —CO₂R⁹ where R⁹ is other than hydrogen or methyl. In other particular embodiments, R³ is —CO₂R⁹ when R¹ is other than lower alkyl, phenyl or substituted phenyl.

Among the aryl nitrone compounds of formulas (I)-(III), there is also a general preference for R¹ to be alkyl, cycloalkyl, aryl or aralkyl, preferably an alkyl and particularly a lower alkyl. Lower alkyls having branching at the 1-position carbon, for example, cyclopropyl, isopropyl, sec-butyl, tert-butyl, cyclobutyl, 1-methylcycloprop-1-yl, sec-pentyl, tert-pentyl, cyclopentyl, 1-methylcyclobut-1-yl and the like are preferred over non-branched equivalents.

There is a preference for R² to be hydrogen, alkyl, heteroalkyl, aralkyl or aryl, with or without further substitution. Hydrogen is a most preferred R² group.

There is a preference for R³ to be —SR⁹, —SO₂NR⁷R⁸, —SO₃R⁹, —CONR⁷—NR⁷R⁸, —OH and —CO₂R⁹. More preferred R³ groups are —SO₂NR⁷R⁸, —SO₃R⁹, —CONR⁷R⁸, and —CO₂R⁹.

There is a preference for the one or more R⁴ groups to be hydrogen.

There is a preference for R⁵ to be hydrogen, —SR⁹, —SO₂NR⁷R⁸, —SO₃R⁹, —CONR⁷R⁸, —NR⁷R⁸, —OH or —CO₂R⁹. More preferred R⁵ groups are hydrogen, —SO₂NR⁷R⁸, —SO₃R⁹, —CONR⁷R⁸ and —CO₂R⁹.

5.4 Heteroaromatic Nitrone Compounds

In additional embodiments, the present invention provides heteroaromatic nitrone compounds according to formula (IV): 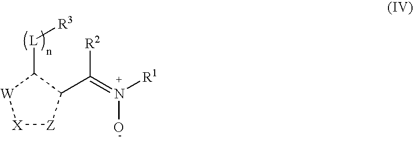
wherein:
W, X and Z are each independently selected from CR⁴, C(R⁴)₂, N, NR⁴, O and S, and form a cycloheteroalkenyl or heteroaryl ring that is substituted only with the -(L)ₙ-R³ and —C(R²)═N(O)—R¹ moieties of formula (IV) or is further substituted;
L is C(R ²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl;
the dotted lines indicate single or double bonds; and
n is an integer from 0 to 1; or
a pharmaceutically acceptable salt or prodrug thereof.

In other embodiments of heteroaromatic nitrones of formula (IV), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In certain embodiments of heteroaromatic nitrone compounds of formula (IV), one of W, X and Z is N. In other embodiments, one of W, X and Z is O. In further embodiments, one of W, X and Z is S.

In other embodiments of nitrone compounds of formula (IV) wherein X, Y and Z form a heteroaryl ring, one of W, X and Z is NR⁴, O or S and the remainder are independently C-R⁴. In further embodiments, one of W and Z is NR⁴, O or S, the other is C—R⁴, and X is C—R⁵, where R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. In certain embodiments R³ and R⁵ are different, while in other embodiments R³ and R⁵ are the same.

In preferred embodiments, the heteroaromatic nitrone compounds according to formula (I), formula (II) or formula (IV) do not encompass any of compounds 51-69. In particular embodiments, the heteroaromatic nitrone compounds according to formula (I), formula (II) or formula (IV) do not encompass any salt of compounds 51-69. In further embodiments, the heteroaromatic nitrone compounds according to formula (I), formula (II) or formula (IV) do not encompass any isomer, diastereomer or enantiomer of compounds 51-69. Compounds 51-69 follow:
51. Nitrone, α-(3-carboxy-2-furyl)-N-[4-(dimethylamino)phenyl]-, methyl ester {or N-[4-(dimethylamino)phenyl]-C-[3-(methoxycarbonyl)furan-2-yl]nitrone};
52. 2,4-Furandisulfonic acid, 5-[[(1-methylethyl)oxidoimino]methyl]-, disodium salt {or N-isopropyl-C-(3,5-disulfofuran-2-yl)nitrone, disodium salt};
53. 1H-Pyrrole-2,4-dicarboxylic acid, 5-[[[4-(dimethylamino)phenyl]oxidoimino] methyl]-3-methyl-, diethyl ester {or N-[4-(dimethylamino)phenyl]-C-[4-methyl-3 ,5-bix(ethoxycarbonyl)-1H-pyrrol-2-yl]nitrone};
54. 3-Thiophenecarboxylic acid, 2-[[[4-(dimethylamino)phenyl]oxidoimino]methyl]-, methyl ester {or N-[4-(dimethylamino)phenyl]-C-[3-(methoxycarbonyl)thiophen-2-yl]nitrone};
55. 5-Pyrimidinecarboxylic acid, 4-[(oxidophenylimino)methyl]-2-phenyl-, ethyl ester {or N-phenyl-C-[5-(ethoxycarbonyl)-2-phenylpyrimidin-4-yl]nitrone};
56. 5-Pyrimidinecarboxylic acid, 4-[[(4-chlorophenyl)oxidoimino]methyl]-2-phenyl-, ethyl ester {or N-(4-chlorophenyl)-C-[5-(ethoxycarbonyl)-2-phenylpyrimidin-4-yl]nitrone};
57. 5-Pyrimidinecarboxylic acid, 4-[[(2-methylphenyl)oxidoimino]methyl]-2-phenyl-, ethyl ester {or N-(2-methylphenyl)-C-[5-(ethoxycarbonyl)-2-phenylpyrimidin-4-yl]nitrone};
58. 5-Pyrimidinecarboxylic acid, 4-[[oxido(phenylmethyl)imino]methyl]-2-phenyl-, ethyl ester {or N-benzyl-C-[5-(ethoxycarbonyl)-2-phenylpyrimidin-4-yl]nitrone};
59. 5-Pyrimidinecarboxylic acid, 4-[[(2-hydroxyethyl)oxidoimino]methyl]-2-phenyl-, ethyl ester {or N-(2-hydroxyethyl)-C-[5-(ethoxycarbonyl)-2-phenylpyrimidin-4-yl]nitrone};
60. 5-Pyrimidinecarboxylic acid, 4-[(butyloxidoimino)methyl]-2-phenyl-, ethyl ester {or N-butyl-C-[5-(ethoxycarbonyl)-2-phenylpyrimidin-4-yl]nitrone};
61. 5-Pyrimidinecarboxylic acid, 4-[(oxidopropylimino)methyl]-2-phenyl-, ethyl ester {or N-propyl-C-[5 -(ethoxycarbonyl)-2-phenylpyrimidin-4-yl]nitrone};
62. 5-Pyrimidinecarboxylic acid, 4-[(ethyloxidoimino)methyl]-2-phenyl-, ethyl ester {or N-ethyl-C-[5-(ethoxycarbonyl)-2-phenylpyrimidin-4-yl]nitrone};
63. 5-Pyrimidinecarboxylic acid, 4-[[[4-(dimethylamino)phenyl]oxidoimino]methyl]-2-phenyl-, ethyl ester {or N-[4-(dimethylamino)phenyl]-C-[5-(ethoxycarbonyl)-2-phenylpyrimidin-4-yl]nitrone};
64. 5-Pyrimidinecarboxylic acid, 4-[N-[p-(dimethylamino)phenyl]formimidoyl]-2-(methylthio)-, ethyl ester, N-oxide {or N-[4-(dimethylamino)phenyl]-C-[5-(ethoxycarbonyl)-2-(methylthio)pyrimidin-4-yl]nitrone};
65. 5-Pyrimidinecarboxylic acid, 4-[(oxidophenylimino)methyl]-2-phenyl-, ethyl ester;
66. 5-Pyrimidinecarboxylic acid, 4-[[[4-(methylsulfonyl)phenyl]oxidoimino]methyl]-2-phenyl-, ethyl ester {or N-[4-(methylsulfonyl)phenyl]-C-[5-(ethoxycarbonyl)-2-phenylpyrimidin-4-yl]nitrone};
67. 1H-Pyrazol-5-amine, 1-[2,6-dichloro-4-(trifluoromethyl)phenyl]-3-[1-(methyloxidoimino)ethyl]-4-(methylthio)-{orN-methyl-C-{5-amino-4-(methylthio)-1-[4-(trifluoromethyl)-2,6-dichlorophenyl]-1H-pyrazol-3-yl}-C ′-(methyl)nitrone};
68. 1H-Pyrrole-3-carboxylic acid, 1-[2-(3,4-dimethoxyphenyl)ethyl]-4,5-dihydro-2-methyl-4-[(methyloxidoimino)methyl]-5-oxo-, methyl ester {or N-methyl-C-[4-(methoxycarbonyl)-5 -methyl-1-[2-(3,4-dimethoxyphenyl)ethyl]-2-oxo-2,3 -dihydro-1H-pyrrol-3-yl]nitrone}; and
69. 3-Furancarboxylic acid, 2-[[[4-dimethylamino)phenyl]oxidoimino]methyl]-5-(hydroxymethyl)-, methyl ester {or N-[4-(dimethylamino)phenyl]-C-[5-(hydroxymethyl)-3-(methoxycarbonyl)furan-2-yl]nitrone}.

In the heteroaromatic nitrone compounds according to formulas (I), (II) and (IV), the rings can be substituted only with the groups depicted in the formulas, or they can be further substituted. For instance, in formula (I), the ring comprising W and Z can be substituted only with the depicted -(L)ₙ-R³ and —C(R²)═N(O)—R¹ moities, or alternatively the ring comprising W and Z can comprise further substituents at any position on the ring. In formulas (II) and (IV), the ring can comprise the two substituents depicted, or the ring can comprise further substituents.

In certain embodiments, R³ is a substituent comprising a sulfur or phosphorus atom. For instance, R³ can be selected from —SO₂NR⁷R⁸, —SO₃R⁹, —PO(OR⁹)NR⁷R⁸, —PO(N⁷R⁸)₂ and —PO(OR⁹)₂.

Among the heteroaromatic nitrone compounds of formulas (I), (II) and (IV), there is a general preference for R¹ to be alkyl, cycloalkyl, aryl or aralkyl, preferably an alkyl and particularly a lower alkyl. Lower alkyls having branching at the 1-position carbon, for example, cyclopropyl, isopropyl, sec-butyl, tert-butyl, cyclobutyl, 1-methylcycloprop-1-yl, sec-pentyl, tert-pentyl, cyclopentyl, 1-methylcyclobut-1-yl and the like are preferred over non-branched equivalents. tert-Butyl is a most preferred R¹ group.

There is a preference for R² to be hydrogen, alkyl, heteroalkyl, aralkyl or aryl, with or without further substitution. Hydrogen is a most preferred R2 group.

There is a preference for R³ to be —SR⁹, —SO₂NR⁷R⁸, —SO₃R⁹, —CONR⁷R⁸, —NR⁷R⁸, —OH or —CO₂R⁹. More preferred R³groups are —SO₂NR⁷R⁸, —SO₃R⁹, —CONR⁷R⁸ and —CO₂R⁹.

There is a preference for the one or more R⁴ groups to be hydrogen.

There is a preference for R⁵ to be hydrogen, —SR⁹, —SO₂NR⁷R⁸, —SO₃R⁹, —CONR⁷R⁸, —NR⁷R⁸, —OH or —CO₂R⁹. More preferred R⁵ groups are hydrogen, —SO₂NR⁷R⁸, —SO₃R⁹, —CONR⁷R⁸ and —CO₂R⁹.

In the heteroaromatic nitrone compounds of the invention, the atom designated by X can be substituted or unsubstituted, especially in compounds where X is a carbon or a heteroatom with a free valence. In certain embodiments, X can be substituted with any group other than hydrogen. For instance, X can be substituted with —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ or —CO₂R⁹.

Referring to heteroaromatic nitrone compounds of formula (II), in some embodiments the six-membered heteroaryl ring contains one nitrogen atom, and in other embodiments the heteroaryl ring contains two nitrogen atoms. In further embodiments the ring contains three nitrogen atoms.

When the heteroaryl ring of formula (II) contains two nitrogen atoms, the two nitrogen atoms can be at any of W, X, Y and Z. For instance, the two nitrogen atoms can be at W and X, at W and Y, at W and Z, at X and Y, at X and Z, or at Y and Z.

In embodiments where X and Z are both N, the other groups of formula (II) can represent particular groups. For instance, Y can be a carbon substituted with any group other than phenyl, substituted phenyl or methylsulfanyl. In other embodiments, Y can be substituted with a group other than sulfanyl, substituted sulfanyl, aryl or substituted aryl. In particular embodiments, Y is substituted with hydrogen, —SO₂NR⁷R⁸, —SO₃R⁹, —CONR⁷R⁸, —NR⁷R⁸, —OH, —PO(OR⁹)NR⁷R⁸, —PO(OR⁹)₂ or —CO₂R⁹. In other embodiments, Y is substituted with —SO₂NR⁷R⁸, —SO₃R⁹, CONR⁷R , —NR⁷R⁸, —OH, —PO(OR⁹)NR⁷R⁸, —PO(OR⁹)₂ or —CO₂R⁹.

In other embodiments where X and Z are both N, R² is selected from substituted or unsubstituted (C₁-C₆)alkyl, substituted or unsubstituted (C₁-C₆)cycloalkyl, substituted or unsubstituted aryl, and substituted or unsubstituted aralkyl. In certain embodiments, the R² group attached to the nitrone group is selected from substituted or unsubstituted (C₁-C₆)alkyl, substituted or unsubstituted (C₁-C₆)cycloalkyl, substituted or unsubstituted aryl, and substituted or unsubstituted aralkyl.

In yet other embodiments where X and Z are both N, R³ is other than —CO₂Et. In further embodiments, R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂ and —PO(OR⁹)₂. In particular embodiments, R³ is —CO₂R⁹ and R⁹ is other than ethyl. In other embodiments, R³ is —CO₂R⁹ and R⁹ is other than lower alkyl.

In further embodiments where two of W, X, Y and Z are N, R¹ is other than phenyl, substituted phenyl or lower alkyl. For instance, R¹ can be substituted or unsubstituted heteroalkyl, substituted or unsubstituted acyl, substituted or unsubstituted heteroaryl, substituted or unsubstituted aralkyl, or substituted or unsubstituted heteroaralkyl.

In still further embodiments of the invention, W and Z join to form a heteroaryl ring of 5 atoms. In certain embodiments, the five-membered ring can comprise a nitrogen atom, an oxygen atom or a sulfur atom. In particular embodiments, the ring can be represented by formula (IV) with groups W, X and Z.

When the heteroaryl ring comprises an oxygen atom, the oxygen atom can be at W, X or Z. In certain embodiments, the oxygen atom is at W or X. In other embodiments, the oxygen atom is at Z. In certain embodiments, Z is oxygen while n is 1.

In certain embodiments, one of W, X and Z is O and the remainder are each independently C—R⁴. In some embodiments, X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. In particular embodiements, R³ and R⁵ are the same.

In further embodiments, Z is O while n is 0. In such embodiments, R² can be selected from substituted or unsubstituted (C₁-C₆)alkyl, substituted or unsubstituted (C₁-C₆)cycloalkyl, substituted or unsubstituted aryl, and substituted or unsubstituted aralkyl. In certain embodiments, the R² group attached to the nitrone group is selected from substituted or unsubstituted (C₁-C₆)alkyl, substituted or unsubstituted (C₁-C₆)cycloalkyl, substituted or unsubstituted aryl, and substituted or unsubstituted aralkyl.

In other embodiments, Z is O while n is 0 and R³ is selected from —SR⁹, —SO₂NR⁷R⁸, —SO₃R⁹, —ONR⁷R⁸, —NR⁷R⁸, —OH, —PO(OR⁹)NR⁷R⁸, —PO(NR⁷R⁸)₂ and -PO(OR⁹)₂. In particular embodiments, R³ is selected from —SR⁹, —SO₂NR⁷R⁸, —CONR⁷R⁸, —NR⁷R⁸, —OH, —PO(OR⁹)NR⁷R⁸, —PO(NR⁷R⁸)₂ and —PO(OR⁹)₂. In other embodiments, R³ is —CO₂R⁹ where R⁹ is other than methyl. In particular embodiments, R⁹ is other than lower alkyl.

In other embodiments, Z is O while n is 0 and R¹ is other than phenyl, substituted phenyl or isopropyl. In particular embodiments, R¹ is other than lower alkyl. For instance, R¹ can be substituted or unsubstituted heteroalkyl, substituted or unsubstituted acyl, substituted or unsubstituted heteroaryl, substituted or unsubstituted aralkyl, or substituted or unsubstituted heteroaralkyl.

In further embodiments, the heteroaryl ring in formula (IV) can comprise a nitrogen atom. The nitrogen atom can be at W, X or Z. In certain embodiments, the nitrogen atom is at W or X. In other embodiments, the nitrogen atom is at Z. In certain embodiments, Z is nitrogen while n is 1. In yet other embodiments, Z is NR⁴.

In certain embodiments, one of W, X and Z is N or NR⁴ and the remainder are each independently selected from C—R⁴, O, S and N. In some embodiments, Z is C—R⁴, O or S.

In further embodiments, Z is N while n is 0. In such embodiments, R² can be selected from substituted or unsubstituted (C₁-C₆)alkyl, substituted or unsubstituted (C₁-C₆)cycloalkyl, substituted or unsubstituted aryl, and substituted or unsubstituted aralkyl. In certain embodiments, the R² group attached to the nitrone group is selected from substituted or unsubstituted (C₁-C₆)alkyl, substituted or unsubstituted (C₁-C₆)cycloalkyl, substituted or unsubstituted aryl, and substituted or unsubstituted aralkyl.

In other embodiments, Z is N while n is 0 and R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂ and —PO(OR⁹)₂. In other embodiments, R³is —CO₂R⁹ where R⁹ is other than ethyl. In particular embodiments, R⁹ is other than lower alkyl.

In other embodiments, Z is N while n is 0 and R¹ is other than phenyl or substituted phenyl. For instance, R¹ can be substituted or unsubstituted aliphatic, substituted or unsubstituted alkyl, substituted or unsubstituted heteroalkyl, substituted or unsubstituted acyl, substituted or unsubstituted heteroaryl, substituted or unsubstituted aralkyl, or substituted or unsubstituted heteroaralkyl.

In further embodiments, Z is N while n is 0 and X is C—R⁴. In these embodiments, R⁴ can be other than methyl, or R⁴ can be other than lower alkyl. For instance, R⁴ can be hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ or —CO₂R⁹.

In other embodiments, the heteroaryl ring in formula (IV) can comprise a sulfur atom. The sulfur atom can be at W, X or Z. In certain embodiments, the sulfur atom is at W or X. In other embodiments, the sulfur atom is at Z. In certain embodiments, Z is sulfur while n is 1.

In certain embodiments, one of W, X and Z is S and the remainder are each independently selected from C—R⁴ and N. In some embodiments, Z is C—R⁴ or N.

In further embodiments, Z is S while n is 0. In such embodiments, R² can be selected from substituted or unsubstituted (C₁-C₆)alkyl, substituted or unsubstituted (C₁-C₆)cycloalkyl, substituted or unsubstituted aryl, and substituted or unsubstituted aralkyl. In certain embodiments, the R group attached to the nitrone group is selected from substituted or unsubstituted (C₁-C₆)alkyl, substituted or unsubstituted (C₁-C₆)cycloalkyl, substituted or unsubstituted aryl, and substituted or unsubstituted aralkyl.

In other embodiments, Z is S while n is 0 and R³is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂ and —PO(OR⁹)₂. In other embodiments, R³ is —CO₂R⁹ where R⁹is other than methyl. In particular embodiments, R⁹ is other than lower alkyl.

In other embodiments, Z is S while n is 0 and R¹ is other than phenyl or substituted phenyl. For instance, R¹ can be substituted or unsubstituted aliphatic, substituted or unsubstituted alkyl, substituted or unsubstituted heteroalkyl, substituted or unsubstituted acyl, substituted or unsubstituted heteroaryl, substituted or unsubstituted aralkyl, or substituted or unsubstituted heteroaralkyl.

In further embodiments, Z is S while n is 0 and X is C—R⁴. In certain embodiments, R⁴ can be other than hydrogen. In other embodiments, R⁴ can be hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ or —CO₂R⁹.

5.5 Bicyclic Aryl Nitrone Compounds

In further embodiments, the present invention provides bicyclic aryl nitrone compounds according to formula (V): 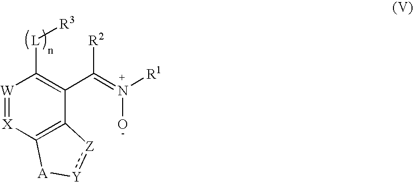
wherein:
W, X and Z are each independently C—R⁴ or C(R⁴)₂;
Y is C—R⁴ or carbonyl;
L is C(R²)₂;
A is selected from NR⁴, O and S;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
the dotted line represents a single or double bond;
or a pharmaceutically acceptable salt or prodrug thereof.

In other embodiments of bicyclic aryl nitrones of formula (V), R³is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In certain embodiments, X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. In other embodiments, R⁵ is other than hydrogen.

In yet other embodiments of bicyclic aryl nitrone compounds of formula (V), Y and Z are each independently selected from C—R⁴, C(R⁴)₂, NR⁴, O, S and carbonyl. In such embodiments, formula (V) includes any arrangements of heteroatoms at positions A, Y and Z that form stable, chemically feasible heterocyclic rings that are recognized by those skilled in the art of organic synthesis. Examples include fused oxazoles, imidazoles and triazoles.

In further embodiments, the present invention provides bicyclic aryl nitrone compounds according to formula (VI): 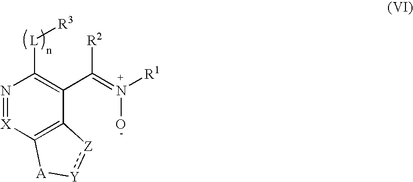
wherein:
X and Z are each independently C—R⁴ or C(R⁴)₂;
Y is C—R⁴ or carbonyl;
L is C(R²)₂;
A is selected from NR⁴, O and S;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(N⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
the dotted line represents a single or double bond; or
a pharmaceutical salt or prodrug thereof.

In other embodiments of bicyclic aryl nitrones of formula (VI), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In certain embodiments, X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. In other embodiments, R⁵ is other than hydrogen.

In other embodiments of bicyclic aryl nitrone compounds of formula (VI), Y and Z are each independently selected from C—R⁴, C(R⁴)₂, NR⁴, O, S and carbonyl. In such embodiments, formula (VI) includes any arrangements of heteroatoms at positions A, Y and Z that form stable, chemically feasible heterocyclic rings that are recognized by those skilled in the art of organic synthesis. Examples include fused oxazoles, imidazoles and triazoles.

In further embodiments, the present invention provides bicyclic aryl nitrone compounds according to formula (VII): 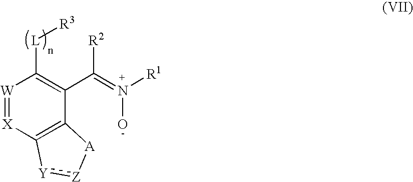
wherein:
W, X and Z are each independently C—R⁴ or C(R⁴)₂;
Y is C—R⁴ or carbonyl;
L is C(R²)₂;
A is selected from NR⁴, O and S;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
the dotted line represents a single or double bond;
or a pharmaceutically acceptable salt or prodrug thereof.

In other embodiments of bicyclic aryl nitrones of formula (VII), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In certain embodiments, X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. In other embodiments, R⁵ is other than hydrogen.

In yet other embodiments of bicyclic aryl nitrone compounds of formula (VII), Y and Z are each independently selected from C—R⁴, C(R⁴)₂, NR⁴, O, S and carbonyl. In such embodiments, formula (VII) includes any arrangements of heteroatoms at positions A, Y and Z that form stable, chemically feasible heterocyclic rings that are recognized by those of skill in the art of organic synthesis. Examples include fused oxazoles, imidazoles and triazoles.

In further embodiments, the present invention provides bicyclic aryl nitrone compounds according to formula (VIII): 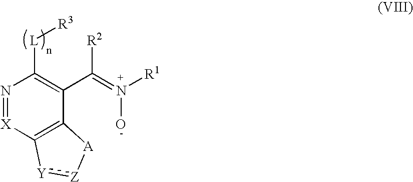
wherein:
X and Z are each independently C—R⁴ or C(R⁴)₂;
Y is C—R⁴ or carbonyl;
L is C(R²)₂;
A is selected from NR⁴, O and S;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(N⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
the dotted line represents a single or double bond;
or a pharmaceutically acceptable salt or prodrug thereof.

In other embodiments of bicyclic aryl nitrones of formula (VIII), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In certain embodiments, X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. In other embodiments, R⁵ is other than hydrogen.

In other embodiments of bicyclic aryl nitrone compounds of formula (VIII), Y and Z are each independently selected from C—R⁴, C(R⁴)₂, NR⁴, O, S and carbonyl. In such embodiments, formula (VIII) includes any arrangements of heteroatoms at positions A, Y and Z that form stable, chemically feasible heterocyclic rings that are recognized by those skilled in the art of organic synthesis. Examples include fused oxazoles, imidazoles and triazoles.

In further embodiments, the present invention provides bicyclic aryl nitrone compounds according to formula (IX): 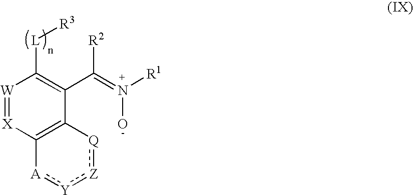
wherein:
W and X are each independently N or C—R⁴;
Y and Z are each independently carbonyl, C—R⁴ or C(R⁴)₂;
L is C(R²)₂;
A and Q are each independently selected from carbonyl, NR⁴, O, S and C—R⁴;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
the dotted lines represent single or double bonds;
or a pharmaceutically acceptable salt or prodrug thereof.

In certain embodiments of bicyclic aryl nitrone compounds of formula (IX), at least one of W and X is N.

In other embodiments of bicyclic aryl nitrones of formula (IX), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In further embodiments, the present invention provides bicyclic aryl nitrone compounds according to formula (X): 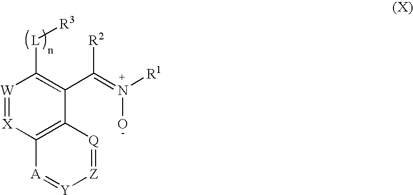
wherein:
W, X, Y, Z, A and Q are each independently selected from N and C—R⁴;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C6)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms; and
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; or
a pharmaceutically acceptable salt or prodrug thereof.

In other embodiments of bicyclic aryl nitrones of formula (X), R³ is selected from —SR⁹ , —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

Referring to bicyclic aryl nitrone compounds of formula (X), in some embodiments the groups W, X, Y and Z are each C—R⁴. When W, X, Y and Z are each C—R⁴, in some embodiments R² is other than hydrogen. In further embodiments when W, X, Y and Z are each C—R⁴, R¹ is other than phenyl, substituted phenyl or methyl. In further embodiments when W, X, Y and Z are each C—R⁴, R¹ is other than phenyl, substituted phenyl or lower alkyl. For instance, R¹ can be substituted or unsubstituted heteroalkyl, substituted or unsubstituted acyl, substituted or unsubstituted heteroaryl, substituted or unsubstituted aralkyl, or substituted or unsubstituted heteroaralkyl.

In further embodiments when W, X, Y and Z are each C—R⁴, R³ is other than —OH or —SMe. For instance, R³ can be selected from —SO₂NR⁷R⁸, —SO₃R⁹, —CONR⁷R⁸, —NR⁷R⁸, —PO(OR⁹)NR⁷R⁸, —PO(NR⁷R⁸)₂, —PO(OR⁹)₂ and —CO₂R⁹. In further embodiments when W, X, Y and Z are each C—R⁴, A and Q are also independently C—R⁴ and X can be substituted with hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ or —CO₂R⁹. In other embodiments, X is substituted with other than hydrogen.

In other embodiments, one of W, X, A, Y, Z and Q is N and the remainder are each independently C—R⁴. In further embodiments, one of A, Y, Z and Q is N and the remainder of that group and W and X are each independently C—R⁴. In particular embodiments, A is N and W, X, Y, Z and Q are each independently C—R⁴. In other particular embodiments, Q is N and W, X, A, Y and Z are each independently C—R⁴. In yet other embodiments, two of W, X, A, Y, Z and Q are N and the remainder are each independently C—R⁴. In further embodiments, two of A, Y, Z and Q are N and the remainder of that group and W and X are each independently C—R⁴. In still other embodiments, three of W, X, A, Y, Z and Q are N and the remainder are each independently C—R⁴.

In further embodiments, the present invention provides bicyclic aryl nitrone compounds according to formula (XI): 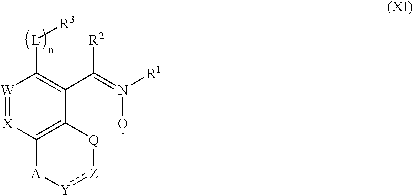
wherein:
W and X are each independently selected from N and C—R⁴;
Y and Z are each independently carbonyl, C—R⁴ or C(R⁴)₂;
A and Q are each independently selected from carbonyl, NR⁴, O and S;
L is C(R²)₂;
R′ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
the dotted line represents a single or double bond;
or a pharmaceutically acceptable salt or prodrug thereof.

In other embodiments of bicyclic aryl nitrones of formula (XI), R³ is selected from —SR⁹, —SO₂NR⁷R⁸, —SO₃R⁹, —CONR⁷R⁸, —NR⁷R⁸, —OH, —PO(OR⁹)NR⁷R⁸, —POR⁷R⁸)₂, —PO(OR⁹)₂ and —CO₂R⁹.

In further embodiments, the present invention provides bicyclic aryl nitrone compounds according to formula (XII): 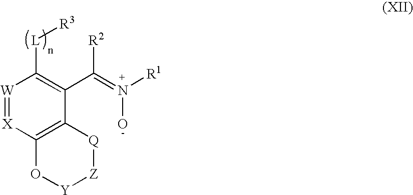
wherein:
W and X are each independently selected from N and C—R⁴;
Q is NR⁴, carbonyl or C(R⁴)₂;
Y and Z are each independently C(R⁴)₂ or carbonyl;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, -PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms; and
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; or
a pharmaceutically acceptable salt or prodrug thereof.

In other embodiments of bicyclic aryl nitrones of formula (XII), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In further embodiments, the present invention provides bicyclic aryl nitrone compounds according to formula (XIII): 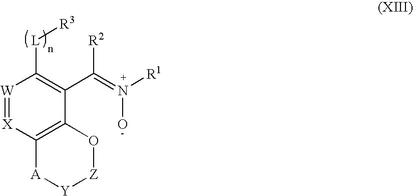
wherein:
W and X are each independently selected from N and C—R⁴;
A is NR⁴, carbonyl or C(R⁴)₂;
Y and Z are each independently C(R⁴)₂ or carbonyl;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms; and
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; or
a pharmaceutically acceptable salt or prodrug thereof.

In other embodiments of bicyclic aryl nitrones of formula (XIII), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In further embodiments, the present invention provides bicyclic aryl nitrone compounds according to formula (XIV): 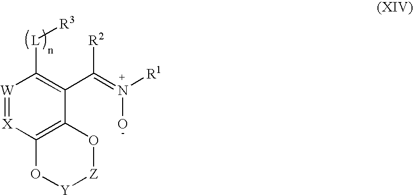
wherein:
W and X are each independently selected from N and C—R⁴;
Y and Z are each independently C(R⁴)₂ or carbonyl;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³is selected from hydrogen, —SR⁹, —SO₂NR⁷R⁸, —SO₃R⁹, —CONR⁷R⁸, —NR⁷R⁸, —OH, —PO(OR⁹)NR⁷R, —PO(NR⁷R⁸)₂, —PO(OR⁹)₂ and —CO₂R⁹;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio;
R⁷ and R8 are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms; and
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; or
a pharmaceutically acceptable salt or prodrug thereof.

In other embodiments of bicyclic aryl nitrones of formula (XIV), R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹.

In other embodiments of bicyclic aryl nitrone compounds of formulas (IX) and (XI)-(XIV), A, Y, Z and Q are each independently selected from carbonyl, C—R⁴, C(R⁴)₂, N, NR⁴, O and S. Such embodiments include those compounds with any arrangements of heteroatoms at positions A, Y, Z and Q that form stable, chemically feasible heterocyclic rings that are recognized by those skilled in the art of organic synthesis.

In yet other embodiments of bicyclic aryl nitrone compounds of each of formulas (V) to (XIV), X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. In yet other embodiments, R⁵ is other than hydrogen.

In preferred embodiments, the bicyclic aryl nitrone compounds according to the formulas above do not encompass any of compounds 70-78. In particular embodiments, the bicyclic aryl nitrone compounds according to the formulas above do not encompass any salt of compounds 70-78. In further embodiments, the bicyclic aryl nitrone compounds according to the formulas above do not encompass any isomer, diastereomer or enantiomer of compounds 70-78. Compounds 70-78 follow:
70. Benzoic acid, 4-[[(2-hydroxy-1-naphthalenyl)methylene]oxidoamino]-, ethyl ester, (Z)-{or (Z)-N-[4-(ethoxycarbonyl)phenyl]-C-(2-hydroxynaphthalen-1-yl)nitrone};
71. 1,4-Benzenediamine, N,N-dimethyl-N′-[[3-(phenylthio)-2-quinoxalinyl]methylene]-, N′-oxide {or N-[4-(dimethylamino)phenyl]-C-[3-(phenylthio)quinoxalin-2-yl]nitrone};
72. Methanamine, N-[[2-(methylthio)-1-naphthalenyl]methylene]-, N-oxide {or N-methyl-C-[2-(methylthio)naphthalen-1-yl]nitrone};
73. Benzenamine, N-[[2-(methylthio)-1-naphthalenyl]methylene]-, N-oxide {or N-phenyl-C-[2-(methylthio)naphthalen-1-yl]nitrone};
74. Benzenamine, 3-methyl-N-[[2-(methylthio)-1-naphthalenyl]methylene]-, N-oxide {or N-(3-methylphenyl)-C-[2-(methylthio)naphthalen-1-yl]nitrone};
75. Benzenamine, 4-methyl-N-[[2-(methylthio)-1-naphthalenyl]methylene]-, N-oxide {or N-(4-methylphenyl)-C-[2-(methylthio)naphthalen-1-yl]nitrone};
76. Benzenamine, 3-chloro-N-[[2-(methylthio)-1-naphthalenyl]methylene]-, N-oxide {or N-(3-chlorophenyl)-C-[2-(methylthio)naphthalen-1-yl]nitrone};
77. Benzenamine, 4-chloro-N-[[2-(methylthio)-1-naphthalenyl]methylene]-, N-oxide {or N-(4-chlorophenyl)-C-[2-(methylthio)naphthalen-1-yl]nitrone}; and
78. 2-Naphthalenol, 1-[[(4-nitrophenyl)oxidoimino]methyl]-{orN-(4-nitrophenyl)-C-(2-hydroxynaphthalen-1-yl)nitrone}.

In the bicyclic aryl nitrone compounds according to the formulas above, the rings can be substituted only with the groups depicted in the formulas, or they can be further substituted. For instance, in formula (I) the ring comprising W and Z can be substituted only with the depicted -(L)ₙ-R³ and -C(R²)═N(O)—R¹ moities, or alternatively the ring comprising W and Z can comprise further substituents at any position on the ring. In the formulas above, the ring can comprise the two substituents depicted, or the ring can comprise further substituents.

Among the bicyclic aryl nitrone compounds described by formula (I), there is a general preference for compounds wherein W and Z are joined to form a 6-membered aryl or heteroaryl ring fused to a 5- or 6-membered cycloalkyl, cycloheteroalkyl, aryl or heteroaryl ring.

Also among the bicyclic aryl nitrone compounds of the formulas above, there is a general preference for R¹ to be alkyl, cycloalkyl, aryl or aralkyl, preferably alkyl and particularly lower alkyl. Lower alkyls having branching at the 1-position carbon, for example, cyclopropyl, isopropyl, sec-butyl, tert-butyl, cyclobutyl, 1-methylcycloprop-1-yl, sec-pentyl, tert-pentyl, cyclopentyl, 1-methylcyclobut-1-yl and the like are preferred over non-branched equivalents. tert-Butyl is a most preferred R¹ group.

There is a preference for R² to be hydrogen, alkyl, heteroalkyl, aralkyl or aryl, with or without further substitution. Hydrogen is a most preferred R² group.

There is a preference for R³ to be —SR⁹, —SO₂NR⁷R⁸, —SO₃R⁹, —CONR⁷R, —NR⁷R⁸, —OH or —CO₂R⁹. More preferred R³ groups are —SO₂NR⁷R⁸, —SO₃R⁹, —CONR⁷R⁸ and CO₂R⁹.

There is a preference for the one or more R⁴ groups to be hydrogen.

There is a preference for R⁵ to be hydrogen, —SR⁹, —SO₂NR⁷R⁸, —SO₃R⁹, —CONR⁷R⁸, —NR⁷R⁸, —OH, —PO(OR⁹)₂ or —CO₂R⁹. More preferred R⁵ groups are hydrogen, —SO₂NR⁷R⁸, —SO₃R⁹, —CONR⁷R⁸ and —CO₂R⁹.

In the bicyclic aryl nitrone compounds of the invention, the atom designated by X can be substituted or unsubstituted, especially in compounds where X is a carbon or a heteroatom with a free valence. In certain embodiments, X can be substituted with any group other than hydrogen. For instance, X can be substituted with hydrogen, —SR⁹, —SO₂NR⁷R⁸, —SO₃R⁹, CONR⁷R⁸, —NR⁷R⁸, —OH, —PO(OR⁹)NR⁷R⁸, —PO(OR⁹)₂ or —CO₂R⁹.

5.6 Derivatives of Aryl, Heteroaromatic and Bicyclic Aryl Nitrone Compounds

In certain aspects, the present invention provides prodrugs and derivatives of: aryl nitrone compounds of formulas (I)-(III); heteroaromatic nitrone compounds of formulas (I), (II) and (IV); and bicyclic aryl nitrone compounds of formulas (I) and (V)-(XIV). Prodrugs are derivatives of the compounds of the invention which have metabolically cleavable groups and become by solvolysis or under physiological conditions the compounds of the invention which are pharmaceutically active in vivo. Such examples include, but are not limited to, choline ester derivatives and the like and N-alkylmorpholine esters and the like.

Other derivatives of the aryl, heteroaromatic and bicyclic aryl nitrone compounds of this invention have activity in both their acid and acid-derivative forms. An acid-sensitive form often offers advantages of solubility, tissue compatibility or delayed release in the mammalian organism (See H. Bundgard, 1985, Design of Prodrugs, Elsevier, Amsterdam, pp. 7-9, 21-24). Prodrugs include acid derivatives well known to practitioners of the art, such as, for example, esters prepared by reaction of the parent acid with a suitable alcohol, amides prepared by reaction of the parent acid compound with a substituted or unsubstituted amine, acid anhydrides and mixed anhydrides. Simple aliphatic or aromatic esters, amides and anhydrides derived from acidic groups pendant on the compounds of this invention are preferred prodrugs. In some cases it is desirable to prepare double ester-type prodrugs such as (acyloxy)alkyl esters or ((alkoxycarbonyl)oxy)alkyl esters. Preferred are the C1-C8 alkyl, C2-C8 alkenyl, aryl, C7-C12 substituted aryl and C7-C12 arylalkyl esters of the compounds of the invention.

5.7 Pharmaceutical Compositions

When employed as pharmaceuticals, the aryl, heteroaromatic and bicyclic aryl nitrone compounds of this invention are typically administered in the form of a pharmaceutical composition. The invention also provides pharmaceutical compositions comprising one or more of: compounds 1, 8, 9, 11, 16-22, 25-27, 37-43 and 45-50 in Section 5.3; compounds 51 and 53-69 in Section 5.4; and compounds 70-78 in Section 5.5. Such compositions can be prepared in a manner well known in the pharmaceutical art and typically comprise a pharmaceutically acceptable carrier and a pharmaceutically effective amount of at least one active compound.

In general, the aryl, heteroaromatic and bicyclic aryl nitrone compounds of this invention are administered in a pharmaceutically effective amount. The amount of the compound actually administered will typically be determined by a physician in light of relevant circumstances, including the condition to be treated, the severity of the patient's symptoms, the chosen route of administration, the actual compound administered, the age, weight, and response of the patient to the treatment, and the like.

The pharmaceutical compositions of this invention can be administered by a variety of routes, including oral, rectal, transdermal, subcutaneous, intravenous, intramuscular and intranasal. Depending on the intended route of delivery, the compounds of this invention are preferably formulated as injectable or oral compositions or, for transdermal administration, as salves, lotions or patches.

The compositions for oral administration can take the form of bulk powders or bulk liquid solutions or suspensions. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules and the like in the case of solid compositions. In such compositions, the active nitrone compound of the invention is usually a minor component (from about 0.1 to about 50% by weight or preferably from about 1 to about 40% by weight) with the remainder being various vehicles or carriers and processing aids helpful for creating the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose; a disintegrating agent such as alginic acid, Primogel or corn starch; a lubricant such as magnesium stearate; a glidant such as colloidal silicon dioxide; a sweetening agent such as sucrose or saccharin; and a flavoring agent such as peppermint, methyl salicylate or orange flavoring.

Injectable compositions are typically based on injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, more preferably from about 0.1 to about 10% by weight, and even more preferably from about 0.5 to about 15% by weight. When formulated as an ointment, the active ingredients will typically be combined with either a paraffinic or a water-miscible ointment base. Alternatively, the active ingredients may be formulated in a cream with, for example, an oil-in-water cream base. Such transdermal formulations are well known in the art and generally include additional ingredients to enhance the dermal penetration or stability of the active ingredients or the formulation. All such known transdermal formulations and ingredients are included within the scope of this invention.

The aryl, heteroaromatic and bicyclic aryl nitrone compounds of this invention can also be administered by a transdermal device. Accordingly, transdermal administration can be accomplished using a patch either of the reservoir or porous membrane type or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference in its entirety.

The aryl, heteroaromatic and bicyclic aryl nitrone compounds of this invention can also be administered in sustained-release forms or from sustained-release drug delivery systems. A description of representative sustained-release materials can be found in Remington's Pharmaceutical Sciences.

The following formulation examples illustrate representative pharmaceutical compositions of this invention. The present invention, however, is not limited to the following pharmaceutical compositions.

### Formulation 1—Tablets

An aryl, heteroaromatic or bicyclic aryl nitrone compound of formula (I) is admixed as a dry powder with a dry gelatin binder in an approximate 1:2 weight ratio. A minor amount of magnesium stearate is added as a lubricant. The mixture is formed into 240-270 mg tablets (80-90 mg of active nitrone compound per tablet) in a tablet press.

### Formulation 2—Tablets

An aryl, heteroaromatic or bicyclic aryl nitrone compound of formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active nitrone compound) in a tablet press.

### Formulation 3—Capsules

An aryl, heteroaromatic or bicyclic aryl nitrone compound of formula (I) is admixed as a dry powder with a starch diluent in an approximate 1:1 weight ratio. The mixture is filled into 250 mg capsules (125 mg of active nitrone compound per capsule).

### Formulation 4—Liquid

An aryl, heteroaromatic or bicyclic aryl nitrone compound of formula (I) (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor and color are diluted with water and added with stirring. Sufficient water is then added to produce a total volume of 5 mL.

### Formulation 5—Injection

An aryl, heteroaromatic or bicyclic aryl nitrone compound of formula (I) is dissolved or suspended in a buffered, sterile, saline, injectable, aqueous medium to a concentration of approximately 5 mg/ml.

### Formulation 6—Topical

Stearyl alcohol (250 g) and a white petrolatum (250 g) are melted at about 75° C. and then a mixture of an aryl, heteroaromatic or bicyclic aryl nitrone compound of formula (I) (50 g), methylparaben (0.25 g), propylparaben (0.15 g), sodium lauryl sulfate (10 g) and propylene glycol (120 g) dissolved in water (about 370 g) is added. The resulting mixture is stirred until it congeals.

### 5.8 Methods of Treatment

The aryl, heteroaromatic and bicyclic aryl nitrone compounds of the present invention are used as therapeutic agents for the treatment of conditions in mammals. Accordingly, the compounds and pharmaceutical compositions of this invention find use as therapeutics for preventing and/or treating pain, neurological and neurodegenerative, autoimmune and inflammatory diseases or conditions in mammals including humans.

In a method of treatment aspect, this invention provides a method of treating a mammal susceptible to or afflicted with a condition associated with arthritis, uveitis, asthma, myocardial infarction, traumatic brain injury, acute spinal cord injury, alopecia (hair loss), inflammatory bowel disease or autoimmune disorders, which method comprises administering an effective amount of one or more of the pharmaceutical compositions described above.

In yet another method of treatment aspect, this invention provides a method of treating a mammal susceptible to or afflicted with a condition that gives rise to pain responses or relates to imbalances in the maintenance of basal activity of sensory nerves. Nitrone compounds have use as analgesics for the treatment of pain of various geneses or etiology, for example, acute inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy and chemotherapy-induced and other iatrogenic neuropathies); visceral pain (such as that associated with gastroesophageal reflex disease, irritable bowel syndrome, inflammatory bowel disease, pancreatitis and various gynecological and urological disorders); dental pain; and headache (such as migraine, cluster headache and tension headache).

In additional method of treatment aspects, this invention provides methods of treating a mammal susceptible to or afflicted with: neurodegenerative diseases and disorders such as, for example, Parkinson's disease, Alzheimer's disease and multiple sclerosis; diseases and disorders which are mediated by or result in neuroinflammation such as, for example, traumatic brain injury, stroke and encephalitis; centrally-mediated neuropsychiatric diseases and disorders such as, for example, depression, mania, bipolar disease, anxiety and schizophrenia; eating disorders, sleep disorders and cognition disorders; epilepsy and seizure disorders; prostate, bladder and bowel dysfunction such as, for example, urinary incontinence, urinary hesitancy, rectal hypersensitivity, fecal incontinence, benign prostatic hypertrophy and inflammatory bowel disease; respiratory and airway diseases and disorders such as, for example, allergic rhinitis, asthma, reactive airway diseases and chronic obstructive pulmonary disease; diseases and disorders which are mediated by or result in inflammation such as, for example, rheumatoid arthritis, osteoarthritis, myocardial infarction, various autoimmune diseases and disorders, uveitis and atherosclerosis; itch/pruritus such as, for example, psoriasis; alopecia (hair loss); obesity; lipid disorders; cancer; high blood pressure; spinal cord injury; and renal disorders. The methods comprise administering an effective condition-treating or condition-preventing amount of one or more of the pharmaceutical compositions described above.

Injection dose levels range from about 0.1 mg/kg/hour to at least 10 mg/kg/hour, all for from about 1 to about 120 hours and especially from 24 to 96 hours. A preloading bolus of from about 0.1 mg/kg to about 10 mg/kg or more may also be administered to achieve adequate steady-state levels. The maximum total dose is not expected to exceed about 2 g/day for a 40 to 80 kg human patient.

For the prevention and/or treatment of long-term conditions, such as neurodegenerative and autoimmune conditions, the regimen for treatment usually stretches over many months or years, so oral dosing is preferred for patient convenience and tolerance. With oral dosing, one to five and especially two to four and typically three oral doses per day are representative regimens. Using these dosing patterns, each dose provides from about 0.01 to about 20 mg/kg of the active nitrone compound, with preferred doses each providing from about 0.1 to about 10 mg/kg and especially from about 1 to about 5 mg/kg.

Transdermal doses are generally selected to provide similar or lower blood levels than those achieved using injection doses.

When used to prevent the onset of a neurodegenerative, autoimmune or inflammatory condition, the nitrone compounds of this invention would be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The aryl, heteroaromatic and bicyclic aryl nitrone compounds of this invention can be administered as the sole active agent or they can be administered in combination with other agents, including other active nitrone compounds.

General Procedures to Synthesize Aryl, Heteroaromatic and Bicyclic Aryl Nitrone Compounds

The aryl, heteroaromatic and bicyclic aryl nitrones of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one skilled in the art by routine optimization procedures.

In addition, conventional protecting groups may be necessary to prevent certain functional groups from undergoing undesired reactions, as will be apparent to those skilled in the art. The choice of a suitable protecting group for a particular functional group as well as suitable conditions for protection and deprotection are well known in the art. For example, numerous protecting groups and the conditions for their introduction and removal are described in T. W. Greene and P. G. M. Wuts, 1991, Protecting Groups in Organic Synthesis, Second Edition, Wiley, New York, and references cited therein.

One known method for preparing nitrones is to react a carboxaldehyde derivative with an appropriately substituted hydroxlamine (or an acid addition salt thereof) and to isolate and purify the product by known standard procedures. Such procedures include, but are not limited to, recrystallization, column chromatography and HPLC.

The reaction of an aromatic aldehyde or ketone with a substituted hydroxylamine (or an acid addition salt thereof) in an organic solvent such as methanol, tetrahydrofuran, dichloromethane, benzene or toluene is known to produce the desired aryl, heteroaromatic or bicyclic aryl nitrone. The reaction may be conducted at ambient temperature or may require heating (e.g., refluxing), and may proceed with or without an organic or inorganic acid as catalyst. Higher temperature may be required when an aromatic ketone is a reactant. The condensation reaction may also be accomplished using microwave-mediated synthesis, which typically employs conditions such as heating to 120° C. for 5-10 min in a sealed tube.

An example of the above-described synthesis of an aryl nitrone is:

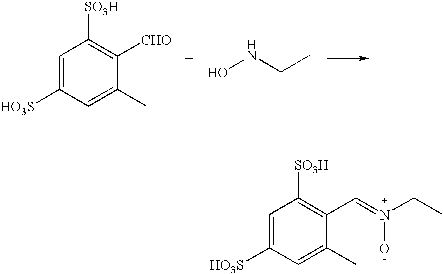

An example of the above-described synthesis of a heteroaromatic nitrone is:

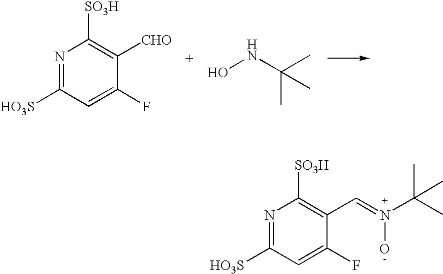

An example of the above-described synthesis of a bicyclic aryl nitrone is:

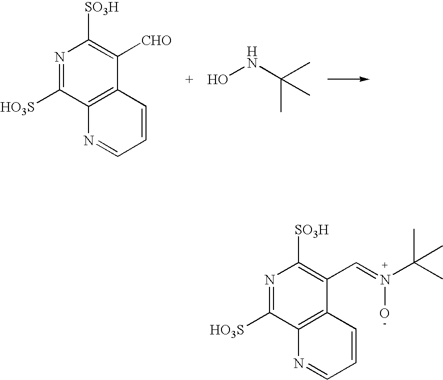

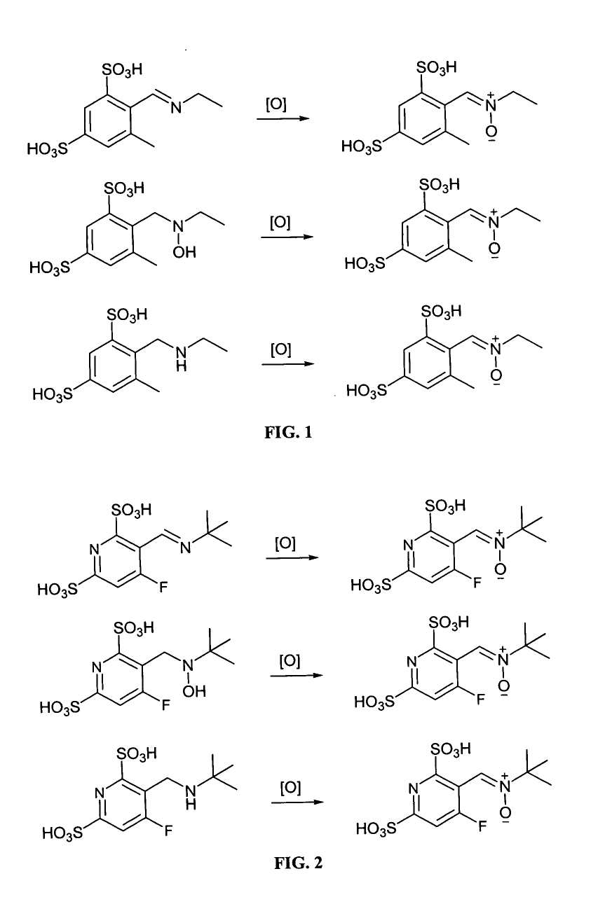

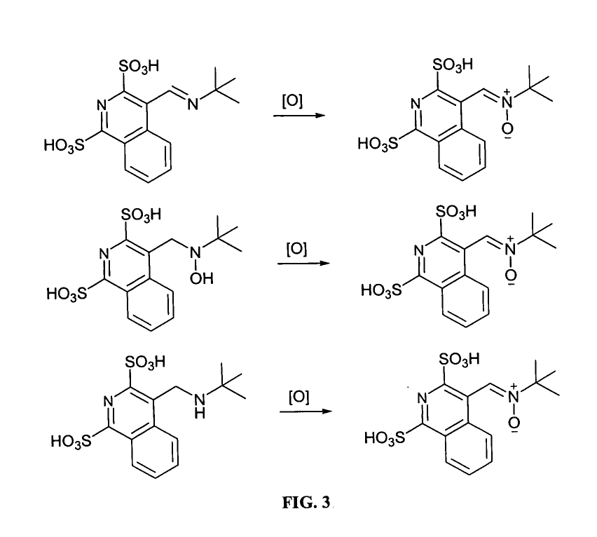

Aryl, heteroaromatic and bicyclic aryl nitrones of the present invention may also be prepared by alternative known methods such as, for example, oxidation of amines, imines or hydroxylamines. FIGS. 1-3 illustrate exemplary oxidative synthetic routes to aryl, heteroaromatic and bicyclic aryl nitrones, respectively.

### A General Procedure for the Sulfonation of Substituted Pyridine/Pyrimidine/Quinoline Aldehyde Derivatives

A substituted pyridine/pyrimidine/quinoline derivative is subjected to neat 30% oleum at temperatures ranging from 50 to 150° C. and the progress of the reaction is monitored by chromatographic or spectroscopic techniques such as TLC, LC/MS or ¹H NMR. Higher temperatures may be employed where multiple sulfonations are desired. Once the reaction is complete, the mixture is cooled to ambient temperature, treated with crushed ice, and carefully made alkaline with 1 M sodium hydroxide solution. The mixture is concentrated in vacuo and the crude product is purified by reverse-phase chromatography using water/acetonitrile containing 1% TFA as eluent.

### A General Procedure for the Amidation of Substituted Pyridine/Pyrimidine/Quinoline Sulfonic Acid Derivatives

To a mixture of a sulfonated derivative in methylene chloride and DMF (1:1) at 0° C. is added oxalyl chloride (1.1 equiv.) slowly dropwise and the mixture is stirred for 4 h. The mixture is re-cooled to 0° C., a solution of an appropriate amine is added, and the mixture is stirred overnight at ambient temperature. Removal of the methylene chloride solvent in vacuo followed by quenching with cold 1 N aqueous HCI affords the product as a precipitate which is filtered, washed with water, and vacuum-dried to obtain the product.

### A General Procedure for the Protection/Esterification of Sulfonic Acid Derivatives

A mixture of a sulfonic acid derivative in acetone is treated with excess anhydrous potassium carbonate at ambient temperature followed by an appropriate alkyl halide. After the mixture is stirred at ambient temperature for 12 h, it is concentrated in vacuo to dryness and the residue is treated with ice-cold water, whereupon the product precipitates out. The non-esterified starting material remains in solution. The precipitated product is filtered, washed with water and vacuum-dried.

### A General Procedure for the Synthesis of Aryl, Heteroaromatic and Bicyclic Aryl Nitrones

A mixture of an appropriate aldehyde or ketone and an appropriate hydroxylamine or acid addition salt thereof (1.5 equiv.) in methanol is stirred at ambient temperature or at elevated (e.g., refluxing) temperature for 6-24 h. Higher temperature may be required when a ketone is a reactant. The progress of the reaction is monitored by chromatographic or spectroscopic techniques such as TLC, LC/MS or ¹H NMR. In some cases excess hydroxylamine or acid addition salt thereof is added to drive the reaction to completion. After the reaction is complete, the mixture is concentrated in vacuo and the crude product is dissolved in ethyl acetate, extracted with water and chromatographed on silica gel to afford the product.

In cases where the sodium salt of a sulfonic acid derivative is desired, the following procedure is employed. The methanolic reaction mixture is set at ambient temperature and treated with sodium methoxide in methanol until the pH of the solution is about 9. Removal of methanol in vacuo followed by precipitation with ether provides the desired sulfonic acid sodium salt derivative.

The following synthetic and biological examples are offered to illustrate this invention and are not to be construed in any way as limiting the scope of this invention.

### 6. EXAMPLES

In the examples below, all temperatures are in degrees Celsius unless otherwise indicated. Examples 1-92 describe the synthesis of various aryl, heteroaromatic and bicyclic aryl nitrones of this invention that have been or could be carried out. The graphical depictions of all the nitrone compounds illustrated herein are not intended to indicate the actual (E)- or (Z)-stereochemistry of the C═N double bond of the nitrone group.

Example 1

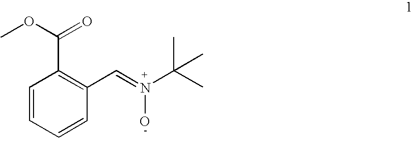

### N-(tert-Butyl)-C-[2-(methoxycarbonyl)phenyl]nitrone (1)

A mixture of commercially available 2-formylbenzoic acid methyl ester (100 mg, 0.61 mmol) and N-(tert-butyl)hydroxylamine hydrochloride (109 mg, 0.732 mmol) in methanol (5 mL) was stirred at ambient temperature for 24 h. The mixture was then concentrated in vacuo and the crude product was dissolved in ethyl acetate (15 ml) and extracted with water (2×20 ml). After the combined organic layers were dried over Na₂SO₄ and concentrated in vacuo, chromatography on silica gel provided compound 1 (10 mg, 20%). MS: m/z 236 (MH+).

Following the procedure described in Example 1, or with slight modifications thereof, and procedures familiar to one of ordinary skill in the art, the compounds of Examples 2-13 and 38-40 were prepared by condensation of appropriate aromatic aldehydes with appropriate hydroxylamines or salts thereof.

### Example 2

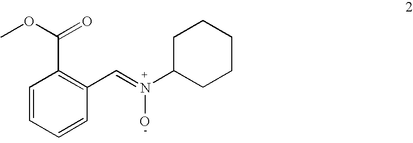

### N-Cyclohexyl-C-[2-(methoxycarbonyl)phenyl]nitrone (2)

Compound 2 was prepared according to the procedure described in Example 1, starting with N-cyclohexylhydroxylamine hydrochloride and methyl 2-formylbenzoate. MS: m/z 262 (MH+).

### Example 3

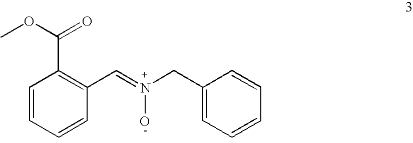

### N-Benzyl-C-[2-(methoxycarbonyl)phenyl]nitrone (3)

Compound 3 was prepared according to the procedure described in Example 1, starting with N-benzylhydroxylamine hydrochloride and methyl 2-formylbenzoate. MS: m/z 270 (MH+).

### Example 4

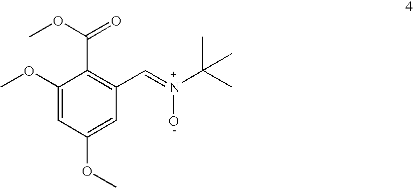

### N-(tert-Butyl)-C-[2-(methoxycarbonyl)-3,5-dimethoxyphenyl]nitrone (4)

Compound 4 was prepared according to the procedure described in Example 1, starting with N-(tert-butyl)hydroxylamine hydrochloride and methyl 2-formyl-4,6-dimethoxybenzoate. MS: m/z 296 (MH+).

### Example 5

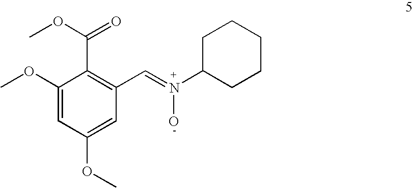

### N-Cyclohexyl-C-[2-(methoxycarbonyl)-3,5-dimethoxyphenyl]nitrone (5)

Compound 5 was prepared according to the procedure described in Example 1, starting with N-cyclohexylhydroxylamine hydrochloride and methyl 2-formyl-4,6-dimethoxybenzoate. MS: m/z 322 (MH+).

### Example 6

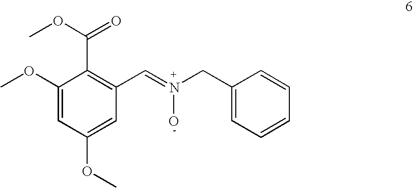

### N-Benzyl-C-[2-(methoxycarbonyl)-3,5-dimethoxyphenyl]nitrone (6)

Compound 6 was prepared according to the procedure described in Example 1, starting with N-benzylhydroxylamine hydrochloride and methyl 2-formyl-4,6-dimethoxybenzoate. MS: m/z 330 (MH+).

### Example 7

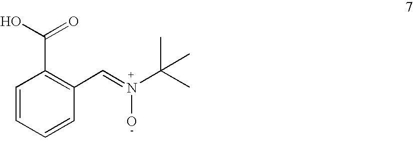

### N-(tert-Butyl)-C-(2-carboxyphenyl)nitrone (7)

Compound 7 was prepared according to the procedure described in Example 1, starting with N-(tert-butyl)hydroxylamine hydrochloride and 2-formylbenzoic acid. MS: m/z 222 (MH+).

### Example 8

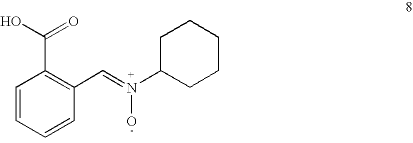

### N-Cyclohexyl-C-(2-carboxyphenyl)nitrone (8)

Compound 8 was prepared according to the procedure described in Example 1, starting with N-cyclohexylhydroxylamine hydrochloride and 2-formylbenzoic acid. MS: m/z 248 (MH+).

### Example 9

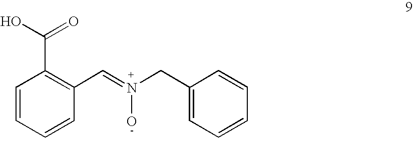

### N-Benzyl-C-(2-carboxyphenyl)nitrone (9)

Compound 9 was prepared according to the procedure described in Example 1, starting with N-benzylhydroxylamine hydrochloride and 2-formylbenzoic acid. MS: m/z 256 (MH+).

### Example 10

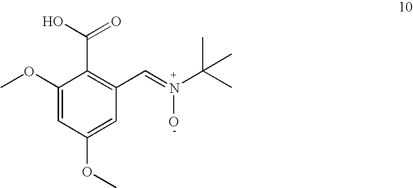

### N-(tert-Butyl)-C-(2-carboxy-3,5-dimethoxyphenyl)nitrone (10)

Compound 10 was prepared according to the procedure described in Example 1, starting with N-(tert-butyl)hydroxylamine hydrochloride and 2-formyl-4,6-dimethoxybenzoic acid. MS: m/z 282 (MH+).

### Example 11

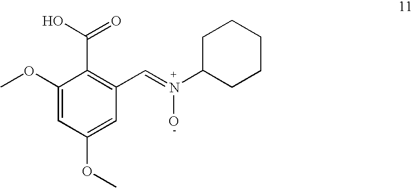

### N-Cyclohexyl-C-(2-carboxy-3,5-dimethoxyphenyl)nitrone (11)

Compound 11 was prepared according to the procedure described in Example 1, starting with N-cyclohexylhydroxylamine hydrochloride and 2-formyl-4,6-dimethoxybenzoic acid. MS: m/z 308 (MH+).

### Example 12

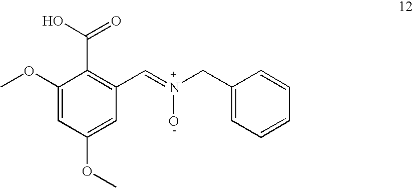

### N-Benzyl-C-(2carbonyl-3,5-dimethozyphenyl)nitrone (12)

Compound 12 was prepared according to the procedure described in Example 1, starting with N-benzylhydroxylamine hydrochloride and 2-formyl-4,6-dimethoxybenzoic acid. MS: m/z 316 (MH+).

### Example 13

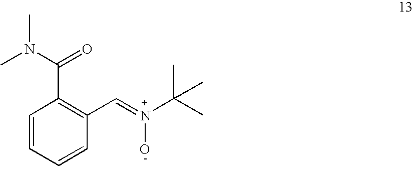

### N-(tert-Butyl)-C-[2-(N,N-dimethylcarbamoyl)phenyl]nitrone (13)

(a) 2-Formyl-N,N-dimethylbenzamide

To a suspension of 2-carboxybenzaldehyde (500 mg, 3.33 mmol) in CH₂Cl₂ (25 ml) was added thionyl chloride (1.98 g, 16.65 mmol) and the mixture was refluxed for 1 h. The resulting solution was then concentrated in vacuo, dissolved in THF, and treated with N,N-dimethylamine (3.9 ml of a 1 M solution in THF, 180 mg, 4.0 mmol) at ice-cold temperature. The mixture was warmed slowly to ambient temperature and stirred at ambient temperature for 2 h. The mixture was then concentrated in vacuo and the crude product was subjected to flash chromatography on silica gel to provide 2-formyl-N,N-dimethylbenzamide (100 mg, 15%). MS: m/z 178 (MH+).

(b) N-(tert-Butyl)-C-[2-(N,N-dimethylcarbamoyl)phenyl]nitrone (13)

Compound 13 was prepared by condensing 2-formyl-N,N-dimethylbenzamide with N-(tert-butyl)hydroxylamine hydrochloride according to the procedure described in Example 1. MS: m/z 249 (MH+).

### Example 14

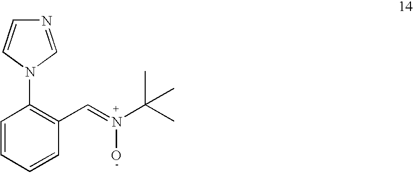

### N-(tert-Butyl)-C-[2-(imidazol-1-yl)phenyl]nitrone (14)

A methanolic solution of 2-(imidazol-1-yl)benzaldehyde (355 mg, 2.03 mmol) and N-(tert-butyl)hydroxylamine hydrochloride (364 mg, 2.44 mmol) was irradiated under microwave at 120° C. for 10 min. The solution was then concentrated in vacuo and the crude product was dissolved in ethyl acetate (20 ml) and extracted with water. After the combined organic layers were dried over Na₂SO₄ and concentrated in vacuo, chromatography on silica gel afforded compound 14 (290 mg, 59%). MS: m/z 244 (MH+).

Following the procedure described in Example 14, or with slight modifications thereof, and procedures familiar to one of ordinary skill in the art, the compounds of Examples 15-37 and 43-53 were prepared by condensation of appropriate aromatic aldehydes with appropriate hydroxylamines or salts thereof.

### Example 15

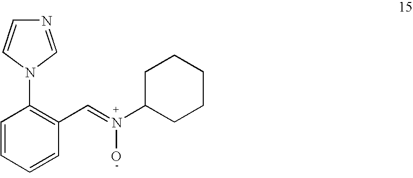

### N-Cyclohexyl-C-[2-(imidazol-1-yl)phenyl]nitrone (15)

Compound 15 was prepared according to the procedure described in Example 14, starting with N-cyclohexylhydroxylamine hydrochloride and 2-(imidazol-1-yl)benzaldehyde. MS: m/z 270 (MH+).

### Example 16

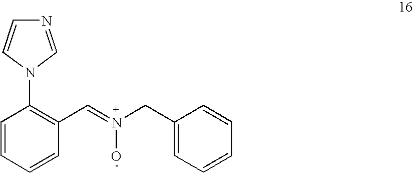

### N-Benzyl-C-[2-(imidazol-1-yl)phenyl]nitrone (16)

Compound 16 was prepared according to the procedure described in Example 14, starting with N-benzylhydroxylamine hydrochloride and 2-(imidazol-1-yl)benzaldehyde. MS: m/z 278 (MH+).

### Example 17

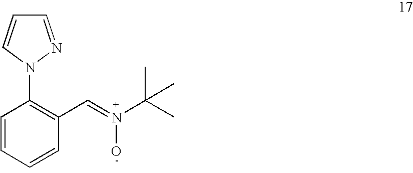

### N-(tert-Butyl)-C-[2-(pyrazol-1-yl)phenyl]nitrone (17)

Compound 17 was prepared according to the procedure described in Example 14, starting with N-(tert-butyl)hydroxylamine hydrochloride and 2-(pyrazol-1-yl)benzaldehyde. MS: m/z 244 (MH+).

### Example 18

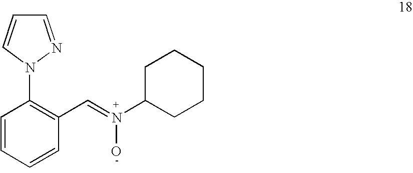

### N-Cyclohexyl-C-[2-(pyrazol-1-yl)phenyl]nitrone (18)

Compound 18 was prepared according to the procedure described in Example 14, starting with N-cyclohexylhydroxylamine hydrochloride and 2-(pyrazol-1-yl)benzaldehyde. MS: m/z 270 (MH+).

### Example 19

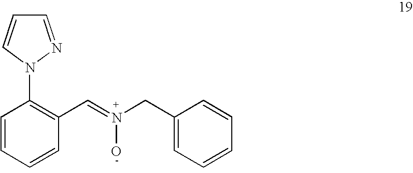

### N-Benzyl-C-[2-(pyrazol-1-yl)phenyl]nitrone (19)

Compound 19 was prepared according to the procedure described in Example 14, starting with N-benzylhydroxylamine hydrochloride and 2-(pyrazol-1-yl)benzaldehyde. MS: m/z 278 (MH+).

### Example 20

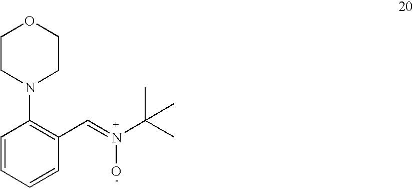

### N-(tert-Butyl)-C-[2-(1-morpholino)phenyl]nitrone (20)

Compound 20 was prepared according to the procedure described in Example 14, starting with N-(tert-butyl)hydroxylamine hydrochloride and 2-(1-morpholino)benzaldehyde. MS: m/z 263 (MH+).

### Example 21

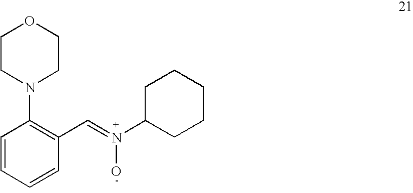

### N-Cyclohexyl-C-[2-(1-morpholino)phenyl]nitrone (21)

Compound 21 was prepared according to the procedure described in Example 14, starting with N-cyclohexylhydroxylamine hydrochloride and 2-(1-morpholino)benzaldehyde. MS: m/z 289 (MH+).

### Example 22

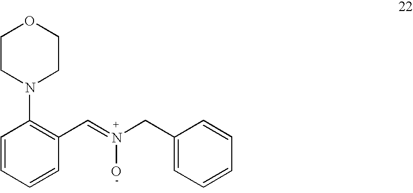

### N-Benzyl-C-[2-(1-morpholino)phenyl]nitrone (22)

Compound 22 was prepared according to the procedure described in Example 14, starting with N-benzylhydroxylamine hydrochloride and 2-(1-morpholino)benzaldehyde. MS: m/z 297 (MH+).

### Example 23

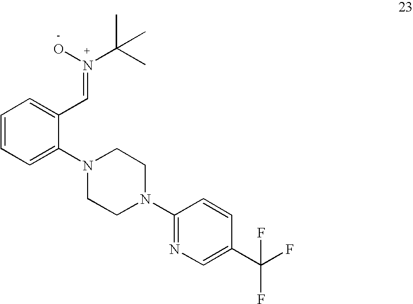

### N-(tert-Butyl)-C-{2-[4-(5-(trifluoromethyl)pyridin-2-yl)piperazin-1-yl]phenyl}nitrone (23)

Compound 23 was prepared according to the procedure described in Example 14, starting with N-(tert-butyl)hydroxylamine hydrochloride and 2-[4-(5-(trifluoromethyl)pyridin-2-yl)piperazin-1-yl]benzaldehyde. MS: m/z 407 (MH+).

### Example 24

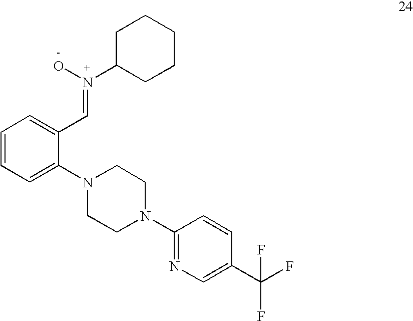

### N-Cyclohexyl-C-{2-[4-(5-(trifluoromethyl)pyridin-2-yl)piperazin-1-yl]phenyl}nitrone (24)

Compound 24 was prepared according to the procedure described in Example 14, starting with N-cyclohexylhydroxylamine hydrochloride and 2-[4-(5-(trifluoromethyl)pyridin-2-yl)piperazin-1-yl]benzaldehyde. MS: m/z 433 (MH+).

### Example 25

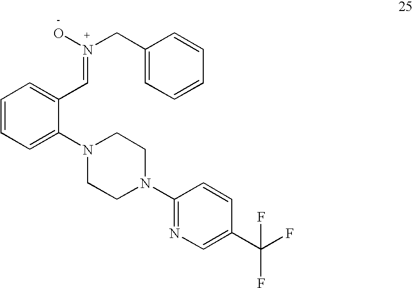

### N-Benzyl-C-{2-[4-(5-(trifluoromethyl)pyridin-2-yl)piperazin-1-yl]phenyl}nitrone (25)

Compound 25 was prepared according to the procedure described in Example 14, starting with N-benzylhydroxylamine hydrochloride and 2-[4-(5-(trifluoromethyl)pyridin-2-yl)piperazin-1-yl]benzaldehyde. MS: m/z 441 (MH+).

### Example 26

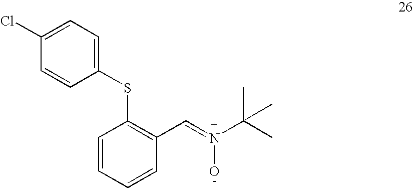

### N-(tert-Butyl)-C-[2-(4-chlorophenylthio)phenyl]nitrone (26)

Compound 26 was prepared according to the procedure described in Example 14, starting with N-(tert-butyl)hydroxylamine hydrochloride and 2-(4-chlorophenylthio)benzaldehyde. MS: m/z 321 (MH+).

### Example 27

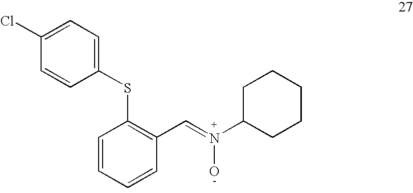

### N-Cycolhexyl-C-[2-(4-chlorophenylthio)phenyl]nitrone (27)

Compound 27 was prepared according to the procedure described in Example 14, starting with N-cyclohexylhydroxylamine hydrochloride and 2-(4-chlorophenylthio)benzaldehyde. MS: m/z 347 (MH+).

### Example 28

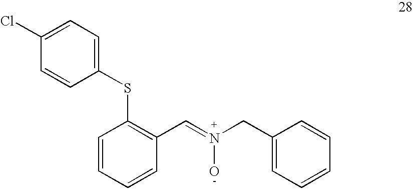

### N-Benzyl-C-[2-(4-chlorophenylthio)phenyl]nitrone (28)

Compound 28 was prepared according to the procedure described in Example 14, starting with N-benzylhydroxylamine hydrochloride and 2-(4-chlorophenylthio)benzaldehyde. MS: m/z 355 (MH+).

### Example 29

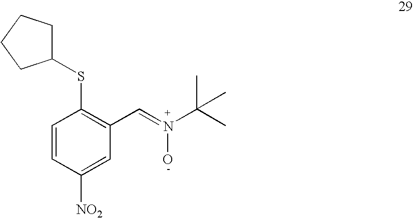

### N-(tert-Butyl)-C-[2-(cyclopentylthio)-5-nitrophenyl]nitrone (29)

Compound 29 was prepared according to the procedure described in Example 14, starting with N-(tert-butyl)hydroxylamine hydrochloride and 2-(cyclopentylthio)-5-nitrobenzaldehyde. MS: m/z 323 (MH+).

### Example 30

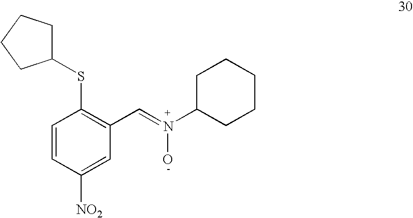

### N-Cyclohexyl-C-[2-(cyclopentylthio)-5-nitrophenyl]nitrone (30)

Compound 30 was prepared according to the procedure described in Example 14, starting with N-cyclohexylhydroxylamine hydrochloride and 2-(cyclopentylthio)-5-nitrobenzaldehyde. MS: m/z 349 (MH+).

### Example 31

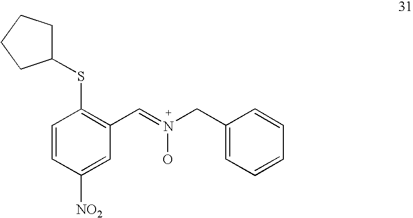

### N-Benzyl-C-[2-(cyclopentylthio)-5-nitrophenyl]nitrone (31)

Compound 31 was prepared according to the procedure described in Example 14, starting with N-benzylhydroxylamine hydrochloride and 2-(cyclopentylthio)-5-nitrobenzaldehyde. MS: m/z 357 (MH+).

### Example 32

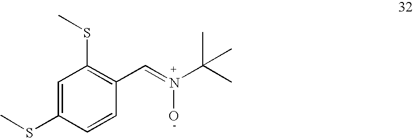

### N-(tert-Butyl)-C-[2,4-bis(methylthio)phenyl]nitrone (32)

Compound 32 was prepared according to the procedure described in Example 14, starting with N-(tert-butyl)hydroxylamine hydrochloride and 2,4-bis(methylthio)benzaldehyde. MS: m/z 270 (MH+); ¹H NMR: (DMSO-d₆) δ 1.5 (s, 9 H), 2.53 (s, 6 H), 7.10 (d, J=6 Hz, 1 H), 7.90 (s, 1 H), 9.11 (d, J=6 Hz, 1 H).

### Example 33

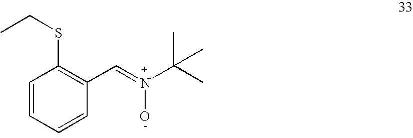

### N-(tert-Butyl)-C-[2-(ethylthio)phenyl]nitrone (33)

Compound 33 was prepared according to the procedure described in Example 14, starting with N-(tert-butyl)hydroxylamine hydrochloride and 2-(ethylthio)benzaldehyde. MS: m/z 238 (MH+); ¹H NMR: (DMSO-d₆) δ 1.7 (t, J=6.0 Hz, 3 H), 1.5 (s, 9 H), 2.9 (q, J=6.0 Hz, 2 H), 7.30 (t, J=6.0 Hz, 1 H), 7.37 (t, J=6.0 Hz, 1 H), 7.52 (d, J=6.0 Hz, 1 H), 8.16 (s, 1 H), 9.13 (d, J=6.0 Hz, 1 H).

### Example 34

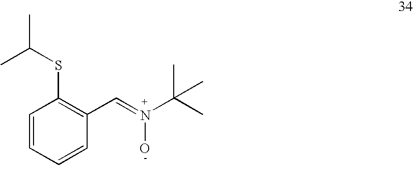

### N-(tert-Butyl)-C-[2-(isopropylthio)phenyl]nitrone (34)

Compound 34 was prepared according to the procedure described in Example 14, starting with N-(tert-butyl)hydroxylamine hydrochloride and 2-(isopropylthio)benzaldehyde. MS: m/z 252 (MH+); ¹H NMR: (DMSO-d₆) δ 1.21 (d, J=6.0 Hz, 6 H), 1.51 (s, 9 H), 3.32 (br s, 1 H), 7.39 (m, 2 H), 7.54(m, 1 H), 8.31 (s, 1 H), 9.20 (m, 1 H).

### Example 35

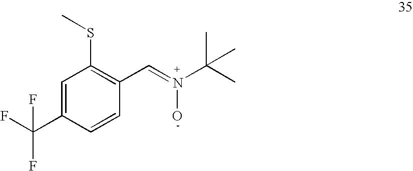

### N-(tert-Butyl)-C-[2-(methylthio)-4-(trifluoromethyl)phenyl]nitrone (35)

Compound 35 was prepared according to the procedure described in Example 14, starting with N-(tert-butyl)hydroxylamine hydrochloride and 2-(methylthio)-4-(trifluoromethyl)benzaldehyde. MS: m/z 292 (MH+); ¹H NMR: (DMSO-d₆) δ 1.5 (s, 9 H), 2.6 (s, 3 H), 7.58 (d, J=6.0 Hz, 1 H), 7.62 (s, 1 H), 8.05 (s, 1 H), 9.18 (d, J=6.0 Hz, 1 H).

### Example 36

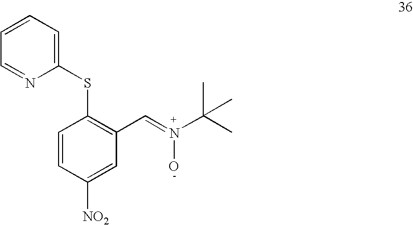

### N-(tert-Butyl)-C-[5-nitro-2-(pyridin-2-ylthio)phenyl]nitrone (36)

Compound 36 was prepared according to the procedure described in Example 14, starting with N-(tert-butyl)hydroxylamine hydrochloride and 5-nitro-2-(pyridin-2-ylthio)benzaldehyde. MS: m/z 332 (MH+).

### Example 37

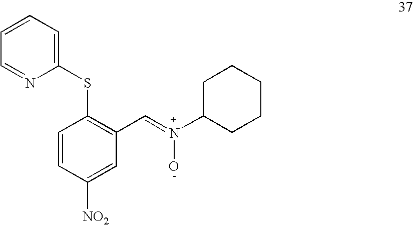

### N-Cyclohexyl-C-[5-nitro-2-(pyridin-2-ylthio)phenyl]nitrone (37)

Compound 37 was prepared according to the procedure described in Example 14, starting with N-cyclohexylhydroxylamine hydrochloride and 5-nitro-2-(pyridin-2-ylthio)benzaldehyde. MS: m/z 358 (MH+).

### Example 38

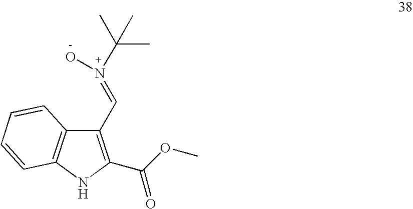

### N-(tert-Butyl)-C-[2-(methoxycarbonyl)-1H-indol-3-yl]nitrone (38)

Compound 38 was prepared according to the procedure described in Example 1, starting with N-(tert-butyl)hydroxylamine hydrochloride and 3-formyl-2-(methoxycarbonyl)indole. MS: m/z 275 (MH+).

### Example 39

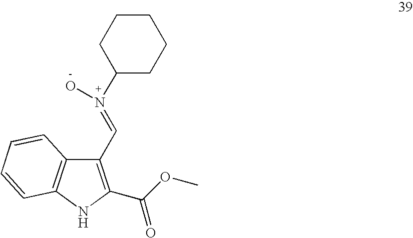

### N-Cyclohexyl-C-[2-(methoxycarbonyl)-1H-indol-3-yl]nitrone (39)

Compound 39 was prepared according to the procedure described in Example 1, starting with N-cyclohexylhydroxylamine hydrochloride and 3-formyl-2-(methoxycarbonyl)indole. MS: m/z 315 (MH+).

### Example 40

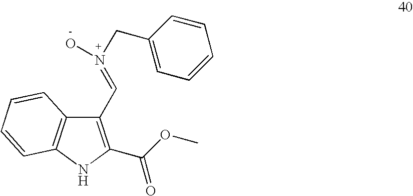

### N-Benzyl-C-[2-(methoxycarbonyl)-1H-indol-3yl]nitrone (40)

Compound 40 was prepared according to the procedure described in Example 1, starting with N-benzylhydroxylamine hydrochloride and 3-formyl-2-(methoxycarbonyl)indole. MS: m/z 309 (MH+).

### Example 41

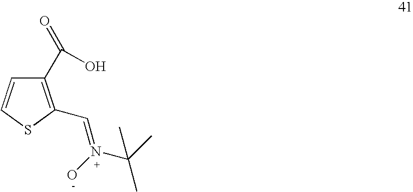

### N-(tert-Butyl)-C-(3-carboxythiophen-2-yl)nitrone (41)

A methanolic solution of 2-formylthiophene-3-carboxylic acid (100 mg, 0.64 mmol) and N-(tert-butyl)hydroxylamine hydrochloride (97.0 mg, 0.77 mmol) was irradiated under microwave at 120° C. for 10 min. The solution was then concentrated in vacuo to dryness. The crude product was crystallized from a mixture of methanol and ethyl acetate to furnish compound 41 (23 mg, 16%). MS: m/z 228 (MH+).

### Example 42

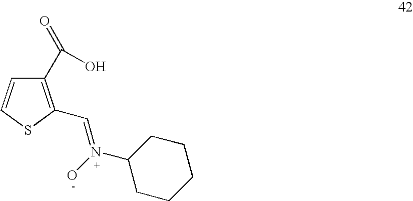

### N-Cyclohexyl-C-(3-carboxythiophen-2-yl]nitrone (42)

Starting with N-cyclohexylhydroxylamine hydrochloride and 2-formylthiophene-3-carboxylic acid, compound 42 was prepared according to the procedure described in Example 41, or with slight modifications thereof as familiar to one of ordinary skill in the art. MS: m/z 254 (MH+).

### Example 43

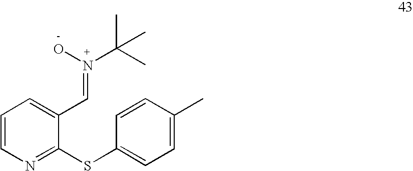

### N-(tert-Butyl)-C-[2-(4-methylphenylthio)pyridin-3-yl]nitrone (43)

Compound 43 was prepared according to the procedure described in Example 14, starting with N-(tert-butyl)hydroxylamine hydrochloride and 2-(4-methylphenylthio)pyridine-3-carboxaldehyde. MS: m/z 301 (MH+).

### Example 44

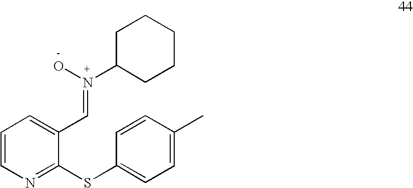

### N-Cyclohexyl-C-[2-(4-methylphenylthio)pyridin-3-yl]nitrone (44)

Compound 44 was prepared according to the procedure described in Example 14, starting with N-cyclohexylhydroxylamine hydrochloride and 2-(4-methylphenylthio)pyridine-3-carboxaldehyde. MS: m/z 327 (MH+).

### Example 45

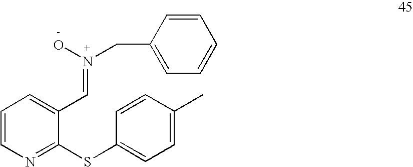

### N-Benzyl-C-[2-(4-methylphenylthio)pyridin-3-yl]nitrone (45)

Compound 45 was prepared according to the procedure described in Example 14, starting with N-benzylhydroxylamine hydrochloride and 2-(4-methylphenylthio)pyridine-3-carboxaldehyde. MS: m/z 335 (MH+).

### Example 46

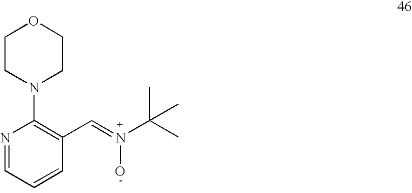

### N-(tert-Butyl)-C-[2-(1-morpholino)pyridin-3-yl9 nitrone (46)

Compound 46 was prepared according to the procedure described in Example 14, starting with N-(tert-butyl)hydroxylamine hydrochloride and 2-(1-morpholino)pyridine-3-carboxaldehyde. MS: m/z 264 (MH+).

### Example 47

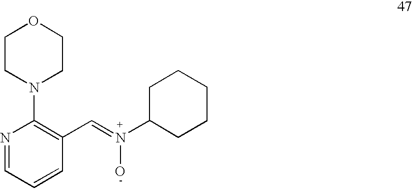

### N-Cyclohexyl-C-[2-(1-morpholino)pyridin-3-yl]nitrone (47)

Compound 47 was prepared according to the procedure described in Example 14, starting with N-cyclohexylhydroxylamine hydrochloride and 2-(1-morpholino)pyridine-3-carboxaldehyde. MS: m/z 290 (MH+).

### Example 48

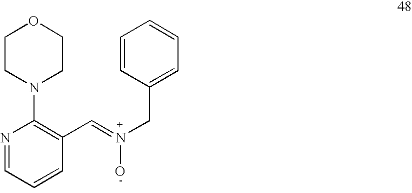

### N-Benzyl-C-[2-(1-morpholino)pyridin-3-yl]nitrone (48)

Compound 48 was prepared according to the procedure described in Example 14, starting with N-benzylhydroxylamine hydrochloride and 2-(1-morpholino)pyridine-3-carboxaldehyde. MS: m/z 298 (MH+).

### Example 49

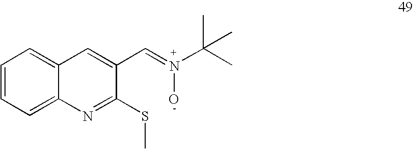

### N-(tert-Butyl)-C-[2-(methylthio)quinolin-3-yl]nitrone (49)

Compound 49 was prepared according to the procedure described in Example 14, starting with N-(tert-butyl)hydroxylamine hydrochloride and 3-formyl-2-(methylthio)quinoline. MS: m/z 275 (MH+).

### Example 50

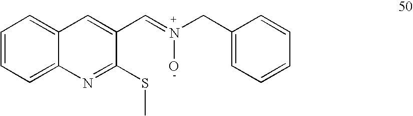

### N-Benzyl-C-[2-(methylthio)quinolin-3-yl]nitrone (50)

Compound 50 was prepared according to the procedure described in Example 14, starting with N-benzylhydroxylamine hydrochloride and 3-formyl-2-(methylthio)quinoline. MS: m/z 309 (MH+).

### Example 51

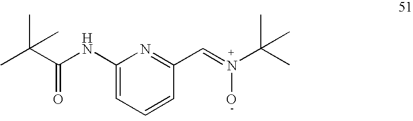

### N-(tert-Butyl)-C-[6-(2,2-dimethylpropanamido)pyridin-2-yl]nitrone (51)

Compound 51 was prepared according to the procedure described in Example 14, starting with N-(tert-butyl)hydroxylamine hydrochloride and 6-(2,2-dimethylpropanamido)pyridine-2-carboxaldehyde. MS: m/z 278 (MH+).

### Example 52

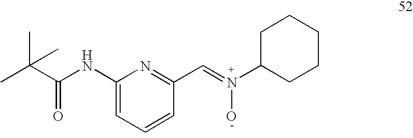

### N-Cyclohexyl-C-[6-(2,2-dimethylpropanamido)pyridin-2-yl]nitrone (52)

Compound 52 was prepared according to the procedure described in Example 14, starting with N-cyclohexylhydroxylamine hydrochloride and 6-(2,2-dimethylpropanamido)pyridine-2-carboxaldehyde. MS: m/z 304 (MH+).

### Example 53

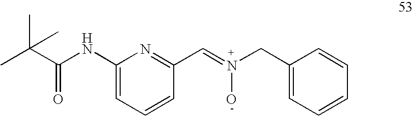

### N-Benzyl-C-[6-(2,2-dimethylpropanamido)pyridin-2-yl]nitrone (53)

Compound 53 was prepared according to the procedure described in Example 14, starting with N-benzylhydroxylamine hydrochloride and 6-(2,2-dimethylpropanamido)pyridine-2-carboxaldehyde. MS: m/z 312 (MH+).

### Example 54

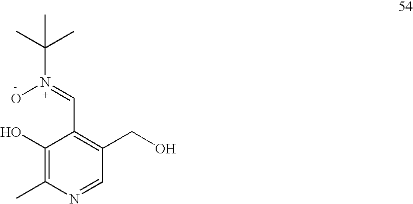

### N-(tert-Butyl)-C-[3-hydroxy-5-(hydroxymethyl)-2-methylpyridin-4-yl]nitrone (54)

A suspension of 3-hydroxy-5-(hydroxymethyl)-2-methylpyridine-4-carboxaldehyde (9.9 g, 49.1 mmol) and N-(tert-butyl)hydroxylamine hydrochloride (9.81 g, 110.0 mmol) in toluene was azeotroped for 22 h using a Dean-Stark apparatus. The mixture was then concentrated in vacuo, giving a yellow precipitate which was crystallized from methanol to afford compound 54 (3.84 g, 33%). ¹H NMR: (CDCl₃) δ 1.65 (s, 9 H), 2.5 (s, 3 H), 4.56 (br s, 2 H), 7.55 (s, 1 H), 8.56 (s, 1 H), 11.45 (br s, 1 H).

### Example 55

### N-Ethyl-C-(2-carboxy-6-methyl-4-sulfophenyl)nitrone

The title compound is prepared following the general procedures described above, starting with N-ethylhydroxylamine hydrochloride and 2-formyl-3-methyl-5-sulfobenzoic acid.

### Example 56

### N-(Tetrahydrofuran-3-yl)-C-(5,6-dichloro-2,4-disulfophenyl)nitrone

The title compound is prepared following the general procedures described above, starting with N-(tetrahydrofuran-3-yl)hydroxylamine hydrochloride and 5,6-dichloro-2,4-disulfobenzaldehyde.

### Example 57

### N-(1,1-Dimethylpropyl)-C-(6-fluoro-2,4-disulfophenyl)nitrone

The title compound is prepared following the general procedures described above, starting with N-(1,1-dimethylpropyl)hydroxylamine hydrochloride and 6-fluoro-2,4-disulfobenzaldehyde.

### Example 58

### N-(1-Methylcyclopropyl)-C-(2,4-disulfophenyl)nitrone

The title compound is prepared following the general procedures described above, starting with N-(1-methylcyclopropyl)hydroxylamine hydrochloride and 2,4-disulfobenzaldehyde.

### Example 59

### N-(2-Hydroxyethyl)-C-(2,4-disulfophenyl)nitrone

The title compound is prepared following the general procedures described above, starting with N-(2-hydroxyethyl)hydroxylamine hydrochloride and 2,4-disulfobenzaldehyde.

### Example 60

### N-(2-Hydroxy-1,1-dimethylethyl)-C-[2,4-bis(N-methylsulfoamido)phenyl]nitrone

The title compound is prepared following the general procedures described above, starting with N-(2-hydroxy-1,1-dimethylethyl)hydroxylamine hydrochloride and 2,4-bis(N-methylsulfoamido)benzaldehyde.

### Example 61

### N-Cyclopentyl-C-(2,4-disulfophenyl)nitrone

The title compound is prepared following the general procedures described above, starting with N-cyclopentylhydroxylamine hydrochloride and 2,4-disulfobenzaldehyde.

### Example 62

### N-Cyclohexyl-C-[2-(O-methylsulfo)phenyl]nitrone

The title compound is prepared following the general procedures described above, starting with N-cyclohexylhydroxylarnine hydrochloride and 2-(O-methylsulfo)benzaldehyde.

### Example 63

### N-Phenyl-C-(2,4-disulfophenyl)nitrone

The title compound is prepared following the general procedures described above, starting with N-phenylhydroxylamine hydrochloride and 2,4-disulfobenzaldehyde.

### Example 64

### N-Benzyl-C-(2,4-diphosphonophenyl)nitrone

The title compound is prepared following the general procedures described above, starting with N-benzylhydroxylamine hydrochloride and 2,4-diphosphonobenzaldehyde.

### Example 65

### N-[(Pyridin-3-yl)methyl]-C-[2,4-bis(phosphonamido)phenyl]nitrone

The title compound is prepared following the general procedures described above, starting with N-[(pyridin-3-yl)methyl]hydroxylamine hydrochloride and 2,4-bis(phosphonamido)benzaldehyde.

### Example 66

### N-(tert-Butyl)-C-[2-(dimethylphospono)phenyl]nitrone

The title compound is prepared following the general procedures described above, starting with N-(tert-butyl)hydroxylamine hydrochloride and 2-(dimethylphospono)benzaldehyde.

### Example 67

### N-Ethyl-C-(6-methyl-2,4-disulfophenyl)nitrone

The title compound is prepared following the general procedures described above, starting with N-ethylhydroxylamine hydrochloride and 6-methyl-2,4-disulfobenzaldehyde.

### Example 68

### N-(tert-Butyl)-C-(2,6-disulfopyridin-3-yl)nitrone

The title compound is prepared following the general procedures described above, starting with N-(tert-butyl)hydroxylamine hydrochloride and 2,6-disulfopyridine-3-carboxaldehyde.

### Example 69

### N-(tert-Butyl)-C-[4,6-bis(O-methylsulfo)pyridin-3-yl]nitrone

The title compound is prepared following the general procedures described above, starting with N-(tert-butyl)hydroxylamine hydrochloride and 4,6-bis(O-methylsulfo)pyridine-3-carboxaldehyde.

### Example 70

### N-(tert-Butyl)-C-[4,6-bis(N-methylsulfoamido)pyridin-3-yl]nitrone

The title compound is prepared following the general procedures described above, starting with N-(tert-butyl)hydroxylamine hydrochloride and 4,6-bis(N-methylsulfoamido)pyridine-3-carboxaldehyde.

### Example 71

### N-(tert-Butyl)-C-(2,5-disulfopyridin-3-yl)nitrone

The title compound is prepared following the general procedures described above, starting with N-(tert-butyl)hydroxylamine hydrochloride and 2,5-disulfopyridine-3-carboxaldehyde.

### Example 72

### N-(tert-Butyl)-C-[4,5-bis(O-methylsulfo)pyridin-3-yl]nitrone

The title compound is prepared following the general procedures described above, starting with N-(tert-butyl)hydroxylamine hydrochloride and 4,5-bis(O-methylsulfo)pyridine-3-carboxaldehyde.

### Example 73

### N-(tert-Butyl)-C-[2,4-bis(N-methylsulfoamido)pyridin-3-yl]nitrone

The title compound is prepared following the general procedures described above, starting with N-(tert-butyl)hydroxylamine hydrochloride and 2,4-bis(N-methylsulfoamido)pyridine-3-carboxaldehyde.

### Example 74

### N-(tert-Butyl)-C-[2,6-bis(O-methoxymethylsulfo)pyridin-3-yl]nitrone

The title compound is prepared following the general procedures described above, starting with N-(tert-butyl)hydroxylamine hydrochloride and 2,6-bis(O-methoxymethylsulfo)pyridine-3-carboxaldehyde.

### Example 75

### N-(tert-Butyl)-C-[4,6-bis(O-methoxymethylsulfo)pyridin-3-yl]nitrone

The title compound is prepared following the general procedures described above, starting with N-(tert-butyl)hydroxylamine hydrochloride and 4,6-bis(O-methoxymethylsulfo)pyridine-3 -carboxaldehyde.

### Example 76

### N-(tert-Butyl)-C-(2-carboxy-6-sulfopyridin-3-yl)-C′-methylnitrone

The title compound is prepared following the general procedures described above, starting with N-(tert-butyl)hydroxylamine hydrochloride and 1-(2-carboxy-6-sulfopyridin-3-yl)ethanone.

### Example 77

### N-(tert-Butyl)-C-(3-carboxy-5-sulfopyridin-2-yl)nitrone

The title compound is prepared following the general procedures described above, starting with N-(tert-butyl)hydroxylamine hydrochloride and 2-formyl-5-sulfopyridine-3-carboxylic acid.

### Example 78

### N-(tert-Butyl)-C-[6-(N,N-dimethylsulfoamido)-4-fluoro-2-sulfopyridin-3-yl]nitrone

The title compound is prepared following the general procedures described above, starting with N-(tert-butyl)hydroxylamine hydrochloride and 6-(N,N-dimethylsulfoamido)-4-fluoro-2-sulfopyridine-3-carboxaldehyde.

### Example 79

### N-(Pyridin-4-yl)-C-[4-chloro-2-(N-methylsulfoamido)-6-(O-methylsulfo)pyridin-3-yl]nitrone

The title compound is prepared following the general procedures described above, starting with N-(pyridin-4-yl)hydroxylamine hydrochloride and 4-chloro-2-(N-methylsulfoamido)-6-(O-methylsulfo)pyridine-3-carboxaldehyde.

### Example 80

### N-(Piperidin-4-yl)-C-[2,4-bis(O-ethylsulfo)-6-(trifluoromethyl)pyrimidin-5-yl]nitrone

The title compound is prepared following the general procedures described above, starting with N-(piperidin-4-yl)hydroxylamine hydrochloride and 2,4-bis(O-ethylsulfo)-6-(trifluoromethyl)pyrimidine-5-carboxaldehyde.

### Example 81

### N-Ethyl-C-(2-carboxy-4-methyl-6-sulfopyridin-3-yl)nitrone

The title compound is prepared following the general procedures described above, starting with N-ethylhydroxylamine hydrochloride and 3-formyl-4-methyl-6-sulfopyridine-2-carboxylic acid.

### Example 82

### N-(Tetrahydrofuran-3-yl)-C-(4,5-dichloro-2,6-disulfopyridin-3-yl)nitrone

The title compound is prepared following the general procedures described above, starting with N-(tetrahydrofuran-3-yl)hydroxylamine hydrochloride and 4,5-dichloro-2,6-disulfopyridine-3-carboxaldehyde.

### Example 83

### N-(tert-Butyl)-C-(4-fluoro-2,6-disulfopyridin-3-yl)nitrone

The title compound is prepared following the general procedures described above, starting with N-(tert-butyl)hydroxylamine hydrochloride and 4-fluoro-2,6-disulfopyridine-3-carboxaldehyde.

### Example 84

### N-(tert-Butyl)-C-(6,8-disulfo-1,7-naphthyridin-5-yl)nitrone

The title compound is prepared following the general procedures described above, starting with N-(tert-butyl)hydroxylamine hydrochloride and 6,8-disulfo-1,7-naphthyridine-5-carboxaldehyde.

### Example 85

### N-(tert-Butyl)-C-(5,7-disulfo-1H-pyrrolo[2,3-c]pyridin-4-yl)nitrone

The title compound is prepared following the general procedures described above, starting with N-(tert-butyl)hydroxylamine hydrochloride and 5,7-disulfo-1H-pyrrolo[2,3-c]pyridin-4-carboxaldehyde.

### Example 86

### N-(tert-Butyl)-C-(2,3-dihydro-6,8-disulfobenzo[b][1,4]dioxin-5-yl)nitrone, disodium salt

The title compound is prepared following the general procedures described above, starting with N-(tert-butyl)hydroxylamine hydrochloride and 8-formyl-2,3-dihydro-benzo[b][1,4]dioxine-5,7-disulfonic acid.

### Example 87

### N-(tert-Butyl)-C-(5-carboxy-7-sulfobenzofuran-4-yl)nitrone

The title compound is prepared following the general procedures described above, starting with N-(tert-butyl)hydroxylamine hydrochloride and 4-formyl-7-sulfobenzofuran-5-carboxylic acid.

### Example 88

### N-(tert-Butyl)-C-{5-(methylamino)-7-(methylcarbamoyl)-3,4-dihydro-2H-pyrano [3,2-c]pyridin-8-yl}nitrone

The title compound is prepared following the general procedures described above, starting with N-(tert-butyl)hydroxylamine hydrochloride and 5-(methylamino)-7-(methylcarbamoyl)-3,4-dihydro-2H-pyrano[3,2-c]pyridine-8-carboxaldehyde.

### Example 89

### N-(tert-Butyl)-C-{7-(N-phenylsulfoamido)-5-sulfo-1H-pyrrolo[2,3-c]pyridin-4-yl}nitrone

The title compound is prepared following the general procedures described above, starting with N-(tert-butyl)hydroxylamine hydrochloride and 7-(N-phenylsulfoamido)-5-sulfo-1 H-pyrrolo [2,3-c]pyridine-4-carboxaldehyde.

### Example 90

### N-(Tetrahydropyran-4-yl)-C-(2,4-disulfonaphthalen-1-yl)nitrone

The title compound is prepared following the general procedures described above, starting with N-(tetrahydropyran-4-yl)hydroxylamine hydrochloride and 4-formylnaphthalene-1,3-disulfonic acid.

### Example 91

### N-(Furan-3-yl)-C-(3-carboxy-1-sulfoisoquinolin-4-yl)nitrone

The title compound is prepared following the general procedures described above, starting with N-(furan-3-yl)hydroxylamine hydrochloride and 4-formyl-1-sulfoisoquinoline-3-carboxylic acid.

### Example 92

### N-(tert-Butyl)-C-(1,3-disulfoisoquinolin-4-yl)nitrone

The title compound is prepared following the general procedures described above, starting with N-(tert-butyl)hydroxylamine hydrochloride and 4-formyl-isoquinoline-1,3-disulfonic acid.

### Example 93

Free Radical-Scavenging/Antioxidant Assay of Nitrone Compounds

Nitrones constitute a chemical class of compounds that have antioxidant properties due to their ability to form stable adducts (i.e., spin traps) with free radicals (See, e.g., Janzen, E.G. et al., 1992, Stabilities of Hydroxyl Radical Spin Adducts of PBN-Type Spin Traps, Free Radical Biol. Med., 12(2): 169-73). Because free radicals can cause oxidative damage to cellular constituents (e.g., proteins and lipids), which can lead to pathological consequences, it has been reported that the antioxidant properties of nitrone compounds at least partly underlie their therapeutic potential, as reported in studies using a canonical member of this chemical class, C-(phenyl)-N-(tert-butyl)nitrone (PBN) (See, e.g., J. M. Carney and R. A. Floyd, 1991, Protection against Oxidative Damage to CNS by α-Phenyl-tert-butylnitrone (PBN) and Other Spin-Trapping Agents: a Novel Series of Nonlipid Free Radical Scavengers, J. Mol. Neurosci., 3(1): 47-57, and Thomas, C.E. et al., 1994, Multiple Mechanisms for Inhibition of Low Density Lipoprotein Oxidation by Novel Cyclic Nitrone Spin Traps, J. Biol. Chem., 269(45): 28055-61).

Therefore, nitrone compounds that have improved antioxidant activity compared to PBN can have better therapeutic potential than PBN. More generally, diseases or conditions that have been reported to be susceptible to antioxidant therapy or that involve the generation of free radicals may be susceptible to nitrone treatment based on the antioxidant activity of nitrones. Diseases or conditions that arise from or are characterized by oxidative damage or oxidative stress include, but are not limited to, neurodegenerative, autoimmune and inflammatory diseases or conditions.

Nitrone compounds of the present invention were tested for their free-radical scavenging/antioxidant activity in an in vitro assay that is accepted by those skilled in the art as a model for conditions involving the generation of free radicals. The assay is based on a reaction between a free-radical donor, 2,2-diphenyl-1-picrylhydrazyl (DPPH), and a radical scavenger/antioxidant to be tested for free-radical scavenging activity. Upon donation of the free-radical electron to the purported radical scavenger, the peak visible absorbance of DPPH (515-520 nm) decreases so that optical density readings at this part of the visual spectrum reflect the progression of the following reaction:
DPPH•+AH=DPPH−H+A•
where AH is a hypothetical radical scavenger/antioxidant. The assay is based on a protocol originally detailed in Brand-Williams, W. et al., 1995, Use of a Free Radical Method to Evaluate Antioxidant Activity, Lebensm. Wiss. Technol., 28:25-30, with further modifications described in L.R. Fukumoto and G. Mazza, 2000, Assessing Antioxidant and Prooxidant Activities of Phenolic Compounds, J. Agric. Food Chem., 48:3597-3604.

The antioxidant assay was performed using Perkin-Elmer 96-well, clear-bottom, black-wall plates (ordered from E & K Scientific Products) and a Tecan Safire absorbance plate reader. The positive controls were Trolox (6-hydroxy-2,5,7,8-tetramethylchromane-2-carboxylic acid, Sigma-Aldrich), BHA (2(3)-tert-butylhydroquinone monomethyl ether, Sigma-Aldrich), PBN (C-(phenyl)-N-(tert-butyl)nitrone, Sigma-Aldrich) and S-PBN (C-(2-sulfophenyl)-N-(tert-butyl)nitrone, sodium salt, prepared according to E. G. Janzen and R. V. Shetty, 1979, Tetrahedron Lett., 35: 3229-32), and the negative control (i.e., vehicle) was DMSO. In brief, 2 μL of 100×DMSO stock of the desired final concentration of each control or nitrone compound to be tested in the same batch was added to a separate well. To each well was then added 198 μL of a freshly made 50 μM DPPH (Sigma-Aldrich) solution in 80% methanol using a multi-channel pipette. The absorbance was immediately read on the plate reader at 520 nm and thereafter read periodically to assess kinetics until all reactios reached completion (i.e., steady state). Since the steady-state point was 24 h, the assay results are shown from the 24 h time point. The absorbance at 520 nm (OD) was plotted versus the concentrations of the controls and nitrone compounds to assess dose-response and interpolated the EC₅₀ values of the controls and test compounds.

In this antioxidant assay, exemplary compounds of the invention exhibited EC₅₀ values as shown in Table 1.

TABLE 1
Free Radical-Scavenging/Antioxidant Activity

| Compound (Name or Example No.) | EC₅₀* |
|---|---|
| Trolox | +++++ |
| BHA | +++++ |
| PBN | + |
| S-PBN | + |
| 1 | ++ |
| 3 | ++ |
| 4 | +++ |
| 5 | +++ |
| 6 | +++ |
| 7 | +++ |
| 8 | ++++ |
| 9 | ++++ |
| 10 | ++ |
| 11 | ++++ |
| 12 | ++++ |
| 13 | +++ |
| 14 | + |
| 15 | + |
| 16 | + |
| 17 | + |
| 18 | + |
| 19 | + |
| 20 | +++ |
| 21 | +++ |
| 22 | + |
| 23 | +++ |
| 24 | ++++ |
| 25 | +++++ |
| 27 | + |
| 28 | +++ |
| 30 | + |
| 31 | ++ |
| 32 | + |
| 33 | + |
| 34 | + |
| 35 | + |
| 36 | +++ |
| 37 | ++++ |
| 38 | + |
| 39 | + |
| 40 | + |
| 41 | ++++ |
| 42 | +++ |
| 43 | + |
| 46 | + |
| 47 | + |
| 48 | + |
| 49 | ++ |
| 51 | + |
| 52 | ++ |
| 53 | ++++ |

*EC₅₀ is the concentration at which a compound reduces by 50% the peak absorbance of DPPH at 520 nm.
+++++ EC₅₀ < 10 μM
++++ 100 μM > EC₅₀ > 10 μM
+++ 500 μM > EC₅₀ > 100 μM
++ 1000 μM > EC₅₀ > 500 μM
+ EC₅₀ > 1000 μM

As can be seen from Table 1, nitrone compounds of the present invention possess significant or potent free-radical scavenging/antioxidant acitivity. Indeed, many of the nitrone compounds of the invention display greater antioxidant activity than PBN. Accordingly, the aryl, heteroaromatic and bicyclic aryl nitrone compounds of the invention are potential therapeutic agents useful for the treatment and/or prevention of diseases or conditions that have been reported to be amenable to antioxidant therapy or involve free-radical generation. Such diseases or conditions include, but are not limited to, pain conditions, autoimmune diseases or conditions, inflammatory diseases or conditions, and neurological or neurodegenerative diseases or conditions.

Non-limiting examples of pain conditions that arise from or are characterized by oxidative damage or oxidative stress are:
migraine (See, e.g., Ciancarelli, I. et al., 2003, Urinary Nitric Oxide Metabolites and Lipid Peroxidation By-Products in Migraine, Cephalalgia, 23(1): 39-42);
acute, chronic and neuropathic pain syndromes and neuralgias (See, e.g., De las Heras Castano, G. et al., 2000, Use of Antioxidants to Treat Pain in Chronic Pancreatitis, Rev. Esp. Enferm. Dig., 92(6): 375-85);
irritable bowel syndrome; and
nerve injury and neuropathies including diabetic neuropathy (See, e.g., Gray, C. et al., 2003, Neuroprotective Effects of Nitrone Radical Scavenger S-PBN on Reperfusion Nerve Injury in Rats, Brain Res., 982(2): 179-85, and Strokov, I. A. et al., 2000, The Function of Endogenous Protective Systems in Patients with Insulin-Dependent Diabetes Mellitus and Polyneuropathy: Effect of Antioxidant Therapy, Bull. Exp. Biol. Med., 130(10): 986-90).

Non-limiting examples of autoimmune diseases or conditions that arise from or are characterized by oxidative damage or oxidative stress are:
multiple sclerosis (See, e.g., Liu, Y. et al., 2003, Bilirubin as a Potent Antioxidant Suppresses Experimental Autoimmune Encephalomyelitis: Implications for the Role of Oxidative Stress in the Development of Multiple Sclerosis, J. Neuroimmunol., 139(1-2): 27-35);
arthritis;
diabetes and related complications (See, e.g., Tabatabaie, T. et al., 1997, Spin Trapping Agent Phenyl-N-tert-butylnitrone Protects against the Onset of Drug-Induced Insulin-Dependent Diabetes Mellitus, FEBS Lett., 407(2): 148-52); and
Graves' disease and other thyroid disorders (See, e.g., Vrca, V. B. et al., 2004, Supplementation with Antioxidants in the Treatment of Graves' Disease: the Effect on Glutathione Peroxidase Activity and Concentration of Selenium, Clin. Chim. Acta., 341(1-2): 55-63).

Non-limiting examples of inflammatory diseases or conditions that arise from or are characterized by oxidative damage or oxidative stress are:
myocardial infarction and dysfunction (See, e.g., Vergely, C. et al., 2003, Effect of Two New PBN-Derived Phosphorylated Nitrones against Postischaemic Ventricular Dysrhythmias, Fundam. Clin. Pharmacol., 17(4): 433-42);
arteriosclerosis and other vascular diseases (See, e.g., Micheletta, F. et al., 2004, Vitamin E Supplementation in Patients with Carotid Atherosclerosis: Reversal of Altered Oxidative Stress Status in Plasma But Not in Plaque, Arterioscler. Thromb. Vasc. Biol., 24(1): 136-40);
asthma, reactive airway diseases and allergies (See, e.g., Nadeem, A. et al., 2003, Increased Oxidative Stress and Altered Levels of Antioxidants in Asthma, J. Allergy Clin. Immunol., 111(1): 72-8);
transplant and graft failure or rejection (See, e.g., Connor, H.D. et aL, 1992, Evidence that Free Radicals Are Involved in Graft Failure following Orthotopic Liver Transplantation in the Rat—an Electron Paramagnetic Resonance Spin Trapping Study, Transplantation, 54(2): 199-204);
lung injury and damage (See, e.g., Murphy, P.G. et al., 1991, Direct Detection of Free Radical Generation in an in vivo Model of Acute Lung Injury, Radical Res. Commun., 15(3): 167-76);
hepatitis and jaundice-induced liver disorders (See, e.g., Yamashita, T. et al., 1996, The Effects of α-Phenyl-tert-butylnitrone (PBN) on Copper-Induced Rat Fulminant Hepatitis with Jaundice, Free Radical Biol. Med., 21(6): 755-61);
pancreatitis and other pancreatic disorders (See, e.g., Koiwai, T. et al., 1989, The Role of Oxygen Free Radicals in Experimental Acute Pancreatitis in the Rat, Int. J. Pancreatol., 5(2): 135-43);
inflammatory bowel disease including Crohn's disease and other disorders of the digestive tract (See, e.g., Reimund, J.M. et al., 1998, Antioxidants Inhibit the in vitro Production of Inflammatory Cytokines in Crohn's Disease and Ulcerative Colitis, Eur. J. Clin. Invest., 28(2): 145-50);
retinal ischemia and damage including macular degeneration and other degenerative or inflammatory disorders of the retina and eye (See, e.g., F. Block and M. Schwarz, 1997, Effects of Antioxidants on Ischemic Retinal Dysfunction, Exp. Eye Res., 64(4): 559-64);
renal ischemia and kidney disorders (See, e.g., Kadkhodaee, M. et al., 1996, Detection of Hydroxyl and Carbon-Centered Radicals by EPR Spectroscopy after Ischaemia and Reperfusion of the Rat Kidney, Free Radical Res., 25(1): 31-42); and
endotoxemia (See, e.g., Harkins, J. D. et al., 1997, Effect of α-Phenyl-tert-butylnitrone on Endotoxin Toxemia in Horses, Vet. Hum. Toxicol., 39(5): 268-71).

Non-limiting examples of neurological or neurodegenerative diseases or conditions that arise from or are characterized by oxidative damage or oxidative stress are:
stroke (See, e.g., Marshall, J. W. et al., 2001, NXY-059, a Free Radical-Trapping Agent, Substantially Lessens the Functional Disability Resulting from Cerebral Ischemia in a Primate Species, Stroke, 32(1): 190-98, and Ginsberg, M. D. et al., 2003, Stilbazulenyl Nitrone, a Novel Antioxidant, Is Highly Neuroprotective in Focal Ischemia, Ann. Neurol., 54(3): 330-42);
schizophrenia and other disorders of cognition (See, e.g., Dakhale, G. et al., 2004, Oxidative Damage and Schizophrenia: the Potential Benefit by Atypical Antipsychotics, Neuropsychobiol., 49(4): 205-09);
mood disorders and other disorders of affect (See, e.g., Ranjekar, P.K. et al., 2003, Decreased Antioxidant Enzymes and Membrane Essential Polyunsaturated Fatty Acids in Schizophrenic and Bipolar Mood Disorder Patients, Psychiatry Res., 121(2): 109-22);
epilepsy (See, e.g., Gupta, M. et al., 2004, Add-on Melatonin Improves Quality of Life in Epileptic Children on Valproate Monotherapy: a Randomized, Double-Blind, Placebo-Controlled Trial, Epilepsy Behav., 5(3): 316-21);
aging and senescence (See, e.g., Carney, J. M. et al., 1991, Reversal of Age-Related Increase in Brain Protein Oxidation, Decrease in Enzyme Activity, and Loss in Temporal and Spatial Memory by Chronic Administration of the Spin-Trapping Compound N-tert-Butyl-α-phenylnitrone, Proc. Natl. Acad. Sci. USA, 88(9): 3633-6);
Parkinson's disease (See, e.g., Fredriksson, A. et al., 1997, MPTP-Induced Deficits in Motor Activity: Neuroprotective Effects of the Spin-Trapping Agent, α-Phenyl-tert-butylnitrone (PBN), J. Neural. Transm., 104(6-7): 579-92);
Alzheimer's disease (See, e.g., Butterfield, D. A. et al., 1996, A β(25-35) Peptide Displays H₂O₂-Like Reactivity towards Aqueous Fe²⁺, Nitroxide Spin Probes, and Synaptosomal Membrane Proteins, Life Sci., 58(3): 217-28);
Huntington's disease (See, e.g., Nakao, N. et al., 1996, Antioxidant Treatment Protects Striatal Neurons against Excitotoxic Insults, Neuroscience, 73(1): 185-200);
amyotrophic lateral sclerosis (See, e.g., Desnuelle, C. et al., 2001, A Double-Blind, Placebo-Controlled Randomized Clinical Trial of α-Tocopherol (Vitamin E) in the Treatment of Amyotrophic Lateral Sclerosis, Amyotrophic Lateral Scler. Other Motor Neuron Disorders, 2(1): 9-18); and
head trauma and traumatic brain injury (See, e.g., Sen, S. et al., 1994, α-Phenyl-tert-butylnitrone Inhibits Free Radical Release in Brain Concussion, Free Radical Biol. Med., 16(6): 685-91, and Marklund, N. et al., 2001, Effects of the Nitrone Radical Scavengers PBN and S-PBN on in vivo Trapping of Reactive Oxygen Species after Traumatic Brain Injury in Rats, J. Cereb. Blood Flow Metab., 21(11); 1259-67).

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A nitrone compound of formula (I): 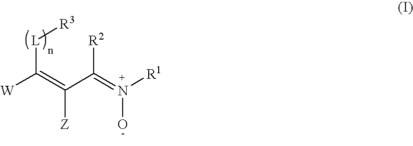
wherein:
W and Z are joined to form a cycloalkenyl or aryl ring of 5 to 8 atoms, and said ring is substituted only with the -(L)ₙ-R³ and —C(R²)═N(O)—R¹ moieties of formula (I) or said ring is further substituted;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from —SR⁹, —SO₂NR⁷R⁸, —SO₃R⁹, —CONR⁷R⁸, —NR⁷R⁸, —PO(OR⁹)NR⁷R⁸, —PO(NR⁷R⁸)₂, —PO(OR⁹)₂ and —CO₂R⁹;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
n is an integer from 0 to 1; or
a pharmaceutically acceptable salt or prodrug thereof;
subject to the proviso that the compound is not selected from the group consisting of compounds 1-50 in Section 5.3.

2. The compound of claim 1, which is an aryl nitrone compound of formula (II): 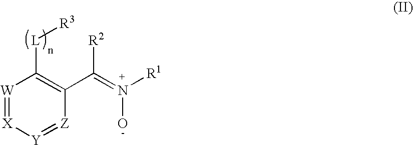
wherein:
each of W, X, Y and Z is independently C—R⁴; and
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio.

3. The compound of claim 1, which is an aryl nitrone compound of formula (III): 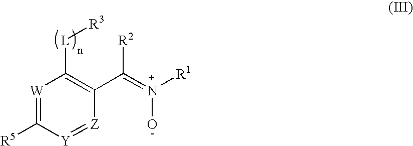
wherein:
each of W, Y and Z is independently C—R⁴;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(N⁷R⁸)₂, —PO(OR⁹)₂ and —CO₂R⁹.

4. The compound of claim 1, wherein said ring is substituted only with the -(L)ₙ-R³ and —C(R²)═N(O)—R¹ moities of formula (I).

5. The compound of claim 1, wherein said ring is further substituted.

6. The compound of claim 3, wherein R³ and R⁵ are identical.

7. The compound of claim 1, 2 or 3, wherein n is 1.

8. The compound of claim 1, 2 or 3, wherein n is 0.

9. The compound of claim 8, wherein R³ is selected from —SO₂NR⁷R⁸, —SO₃R⁹, —PO(OR⁹)NR⁷R⁸, —PO(NR⁷R⁸)₂ and —PO(OR⁹)₂.

10. The compound of claim 8, wherein R³ is —SO₃H and R¹ is not lower alkyl, acetylated lower alkyl, hydroxylated lower alkyl, phenyl or substituted phenyl.

11. The compound of claim 8, wherein R³ is —SR⁹ and R⁹ is other than phenyl or methyl.

12. The compound of claim 8, wherein R³ is —SR⁹ and R¹ is other than —CH₂(C₆H₅), substituted —CH₂(C₆H₅), cyclohexyl, substituted cyclohexyl, methyl, phenyl or substituted phenyl.

13. The compound of claim 8, wherein R³ is —OH and R¹ is other than —CH₂(C₆H₅), substituted —CH₂(C₆H₅), lower alkyl, phenyl or substituted phenyl.

14. The compound of claim 8, wherein R³ is —NR⁷R⁸, and R⁷ and R⁸ are both other than hydrogen.

15. The compound of claim 8, wherein R³ is —NR⁷R⁸ and R¹ is other than —CH₂CO₂Me, —CH₂CO₂H, —CH₂(C₆H₅), substituted —CH₂(C₆H₅), lower alkyl, phenyl or substituted phenyl.

16. The compound of claim 8, wherein R³ is —CO₂R⁹ and R⁹ is other than hydrogen or methyl.

17. The compound of claim 8, wherein R³ is —CO₂R⁹ and R¹ is other than lower alkyl, phenyl or substituted phenyl.

18. A nitrone compound of formula (I): 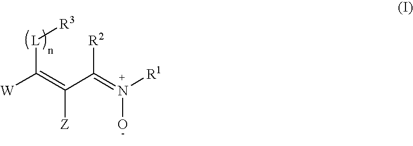
wherein:
W and Z are joined to form a cycloheteroalkenyl or heteroaryl ring of 5 to 8 atoms, and said ring is substituted only with the -(L)ₙ-R³ and —C(R²)═N(O)—R¹ moieties of formula (I) or said ring is further substituted; or
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂, —PO(OR⁹)₂ and —CO₂R⁹;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
n is an integer from 0 to 1; or
a pharmaceutically acceptable salt or prodrug thereof;
subject to the proviso that the compound is not selected from the group consisting of compounds 51-69 in Section 5.4.

19. The compound of claim 18, which is an aryl nitrone compound of formula (II): 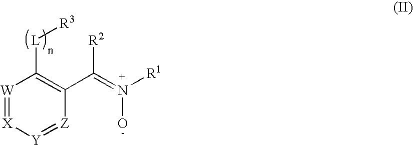
wherein:
m of W, X, Y and Z is N and the remainder are each independently C—R⁴;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
m is an integer from 1 to 3.

20. The aryl nitrone compound of claim 19, wherein X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

21. The aryl nitrone compound of claim 19, wherein one of W, X, Y, and Z is N and the remainder are each independently C—R⁴.

22. The aryl nitrone compound of claim 21, wherein X is N.

23. The aryl nitrone compound of claim 21, wherein W is N.

24. The aryl nitrone compound of claim 21, wherein Y is N.

25. The aryl nitrone compound of claim 21, wherein Z is N.

26. The aryl nitrone compound of claim 23, 24 or 25, wherein X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

27. The aryl nitrone compound of claim 26, wherein R³ and R⁵ are the same.

28. The aryl nitrone compound of claim 19, wherein two of W, X, Y, and Z are N.

29. The aryl nitrone compound of claim 28, wherein W and X are each N.

30. The aryl nitrone compound of claim 28, wherein X and Y are each N.

31. The aryl nitrone compound of claim 28, wherein X and Z are each N.

32. The aryl nitrone compound of claim 28, wherein W and Y are each N.

33. The aryl nitrone compound of claim 28, wherein W and Z are each N.

34. The aryl nitrone compound of claim 28, wherein Y and Z are each N.

35. The aryl nitrone ompound of claim 32, 33 or 34, wherein X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

36. The aryl nitrone compound of claim 35, wherein R³ and R⁵ are the same.

37. The aryl nitrone compound of claim 19 or 31, wherein Y is C—R⁶ and R⁶ is:
hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio, but not phenyl, substituted phenyl or —SMe.

38. The aryl nitrone compound of claim 37, wherein R² is selected from substituted or unsubstituted (C₁-C₆)alkyl, substituted or unsubstituted (C₁-C₆)cycloalkyl, substituted or unsubstituted aryl, and substituted or unsubstituted aralkyl.

39. The aryl nitrone compound of claim 37, wherein R⁶ is other than alkylthio.

40. The aryl nitrone compound of claim 37, wherein R⁶ is other than sulfanyl or substituted sulfanyl.

41. The aryl nitrone compound of claim 37, wherein R⁶ is other than aryl.

42. The aryl nitrone compound of claim 37, wherein R⁶ is other than aryl or substituted aryl.

43. The aryl nitrone compound of claim 37, wherein R⁶ is selected from hydrogen, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

44. The aryl nitrone compound of claim 43, wherein R⁶ is selected from —SO₂NR⁷R⁸, —SO₃R⁹, —CONR⁷R⁸, —NR⁷R⁸, —OH, —PO(OR⁹)NR⁷R, —PO(OR⁹)₂ and —CO₂R⁹.

45. The aryl nitrone compound of claim 37, wherein R³ is other than —CO₂Et.

46. The aryl nitrone compound of claim 37, wherein R³ is selected from —SR⁹, —SO₂NR⁷R⁸, —SO₃R⁹, CONR⁷R⁸, —NR⁷R⁸, —OH, —PO(OR⁹)NR⁷R⁸, —PO(NR⁷R⁸)₂ and —PO(OR⁹)₂.

47. The aryl nitrone compound of claim 37, wherein R³ is —CO₂R⁹ and R⁹ is other than ethyl.

48. The aryl nitrone compound of claim 37, wherein R³ is —CO₂R⁹ and R⁹ is other than lower alkyl.

49. The aryl nitrone compound of claim 37, wherein R¹ is other than phenyl, substituted phenyl or lower alkyl.

50. The aryl nitrone compound of claim 37, wherein R¹ is substituted or unsubstituted heteroalkyl, substituted or unsubstituted acyl, substituted or unsubstituted heteroaryl, substituted or unsubstituted aralkyl, or substituted or unsubstituted heteroaralkyl.

51. The aryl nitrone compound of claim 19, wherein m=3.

52. The compound of claim 18, wherein W and Z are joined to form a substituted or unsubstituted heteroaryl ring of 5 atoms, and said ring is substituted only with the -(L)₁-R³ and —C(R²)═N(O)—R¹ moieties of formula (I) or said ring is further substituted.

53. The compound of claim 18, which is a cycloheteroalkenyl or heteroaryl nitrone compound of formula (IV): 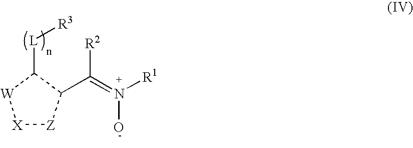
wherein:
W, X and Z are each independently selected from CR⁴, C(R⁴)₂, N, NR⁴, O and S, and form a cycloheteroalkenyl or heteroaryl ring that is substituted only with the -(L)ₙ-R³ and —C(R²)═N(O)—R¹ moieties of formula (IV) or is further substituted;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted lines indicate single or double bonds.

54. The heteroaryl nitrone compound of claim 53, wherein one of W, X and Z is O and the remainder are each independently C—R⁴.

55. The heteroaryl nitrone compound of claim 54, wherein X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

56. The heteroaryl nitrone compound of claim 55, wherein R³ and R⁵ are the same.

57. The heteroaryl nitrone compound of claim 54, wherein W or X is O.

58. The heteroaryl nitrone compound of claim 54, wherein Z is O and n=1.

59. The heteroaryl nitrone compound of claim 54, wherein Z is O and R² is selected from substituted or unsubstituted (C₁-C₆)alkyl, substituted or unsubstituted (C₁-C₆)cycloalkyl, substituted or unsubstituted aryl, and substituted or unsubstituted aralkyl.

60. The heteroaryl nitrone compound of claim 54, wherein Z is O and R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂ and —PO(OR⁹)₂.

61. The heteroaryl nitrone compound of claim 54, wherein Z is O and R³ is selected from —SR⁹, —SO₂NR⁷R, —CONR⁷R⁸, —NR⁷R⁸, —OH, —PO(OR⁹)NR⁷R⁸, —PO(NR⁷R⁸)₂ and —PO(OR⁹)₂.

62. The heteroaryl nitrone compound of claim 54, wherein Z is O, R³ is —CO₂R⁹, and R⁹ is other than methyl.

63. The heteroaryl nitrone compound of claim 62, wherein R⁹is other than lower alkyl.

64. The heteroaryl nitrone compound of claim 54, wherein Z is O and R¹ is other than phenyl, substituted phenyl or isopropyl.

65. The heteroaryl nitrone compound of claim 64, wherein R¹ is other than lower alkyl.

66. The heteroaryl nitrone compound of claim 54, wherein Z is O and R¹ is substituted or unsubstituted heteroalkyl, substituted or unsubstituted acyl, substituted or unsubstituted heteroaryl, substituted or unsubstituted aralkyl, or substituted or unsubstituted heteroaralkyl.

67. The heteroaryl nitrone compound of claim 54, wherein Z is O, X is C—R⁵, and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, ——PO(OR⁹)₂ and —CO₂R⁹.

68. The heteroaryl nitrone compound of claim 53, wherein one of W, X and Z is N or NR⁴ and the remainder are each independently selected from C—R⁴, O, S and N.

69. The heteroaryl nitrone compound of claim 68, wherein W or X is N.

70. The heteroaryl nitrone compound of claim 68, wherein Z is C—R⁴, O or S.

71. The heteroaryl nitrone compound of claim 68, wherein Z is N and n=1.

72. The heteroaryl nitrone compound of claim 68, wherein Z is N and R² is selected from substituted or unsubstituted (C₁-C₆)alkyl, substituted or unsubstituted (C₁-C₆)cycloalkyl, substituted or unsubstituted aryl, and substituted or unsubstituted aralkyl.

73. The heteroaryl nitrone compound of claim 68, wherein Z is N and R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂ and —PO(OR⁹)₂.

74. The heteroaryl nitrone compound of claim 68, wherein Z is N, R³ is —CO₂R⁹, and R⁹ is other than ethyl.

75. The heteroaryl nitrone compound of claim 74, wherein R⁹ is other than lower alkyl.

76. The heteroaryl nitrone compound of claim 68, wherein Z is N and R¹ is other than phenyl or substituted phenyl.

77. The heteroaryl nitrone compound of claim 68, wherein Z is N and R¹ is substituted or unsubstituted aliphatic, substituted or unsubstituted alkyl, substituted or unsubstituted heteroalkyl, substituted or unsubstituted acyl, substituted or unsubstituted heteroaryl, substituted or unsubstituted aralkyl, or substituted or unsubstituted heteroaralkyl.

78. The heteroaryl nitrone compound of claim 68, wherein Z is N, X is C—R⁴, and R⁴ for X is other than methyl.

79. The heteroaryl nitrone compound of claim 68, wherein Z is N, X is C—R⁴, and R⁴ for X is other than lower alkyl.

80. The heteroaryl nitrone compound of claim 68, wherein Z is N, X is C—R⁵, and R⁵ is selected from hydrogen, —SR⁹, —SO₂NR⁷R⁸, —SO₃R⁹, —CONR⁷R⁸, —NR⁷R⁸, —PO(OR⁹)NR⁷R⁸, —PO(OR⁹)₂ and —CO₂R⁹.

81. The heteroaryl nitrone compound of claim 53, wherein one of W, X and Z is S and the remainder are each independently selected from C—R⁴ and N.

82. The heteroaryl nitrone compound of claim 81, wherein W or X is S.

83. The heteroaryl nitrone compound of claim 81, wherein Z is C—R⁴ or N.

84. The heteroaryl nitrone compound of claim 81, wherein Z is S and n=1.

85. The heteroaryl nitrone compound of claim 81, wherein Z is S and R² is selected from substituted or unsubstituted (C₁-C₆)alkyl, substituted or unsubstituted (C₁-C₆)cycloalkyl, substituted or unsubstituted aryl, and substituted or unsubstituted aralkyl.

86. The heteroaryl nitrone compound of claim 81, wherein Z is S and R³ is selected from —SR⁹, —SO₂NR⁷R⁸, —SO₃R⁹, —CONR⁷R⁸, —NR⁷R⁸, —OH, —PO(OR⁹)NR⁷R⁸, —PO(NR⁷R⁸)₂ and —PO(OR⁹)₂.

87. The heteroaryl nitrone compound of claim 81, wherein Z is S, R³ is —CO₂R⁹, and R⁹ is other than methyl.

88. The heteroaryl nitrone compound of claim 87, wherein R⁹ is other than lower alkyl.

89. The heteroaryl nitrone compound of claim 81, wherein Z is S and R¹ is other than phenyl or substituted phenyl.

90. The heteroaryl nitrone compound of claim 81, wherein Z is S and R¹ is substituted or unsubstituted aliphatic, substituted or unsubstituted alkyl, substituted or unsubstituted heteroalkyl, substituted or unsubstituted acyl, substituted or unsubstituted heteroaryl, substituted or unsubstituted aralkyl, or substituted or unsubstituted heteroaralkyl.

91. The heteroaryl nitrone compound of claim 81, wherein Z is S, X is C—R⁴, and R⁴ for X is other than hydrogen.

92. The heteroaryl nitrone compound of claim 81, wherein Z is S, X is C—R⁵, and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

93. A nitrone compound of formula (I): 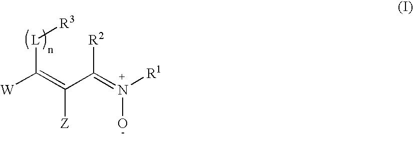
wherein:
W and Z are joined to form a bicycloalkenyl, bicycloheteroalkenyl, bicycloaryl or bicycloheteroaryl ring of 8 to 11 atoms, and said ring is substituted only with the -(L)ₙ-R³ and —C(R²)═N(O)R¹ moieties of formula (I) or said ring is further substituted;
L is C(R²)₂;
R¹ is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted acyl, substituted or unsubstituted aryl, substituted or unsubstituted heteroaryl, substituted or unsubstituted aralkyl, and substituted or unsubstituted heteroaralkyl;
each R² is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl;
R³ is selected from —SR⁹, —SO₂NR⁷R⁸, —SO₃R⁹, —CONR⁷R⁸, —NR⁷R⁸, —PO(OR⁹)NR⁷R⁸, —PO(NR⁷R⁸)₂, —PO(OR⁹)₂ and —CO₂R⁹;
R⁷ and R⁸ are each independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, substituted or unsubstituted heteroaralkyl, —CO₂R⁹ and —CON(R⁹)₂, and may join together to form a substituted or unsubstituted heteroaryl ring or a saturated or unsaturated substituted or unsubstituted cycloheteroalkyl ring of 4 to 7 atoms;
each R⁹ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted acyl, substituted or unsubstituted aryl, substituted or unsubstituted heteroaryl, substituted or unsubstituted aralkyl, and substituted or unsubstituted heteroaralkyl; and
n is an integer from 0 to 1; or
a pharmaceutically acceptable salt or prodrug thereof;
subject to the proviso that the compound is not selected from the group consisting of compounds 70-78 in Section 5.5.

94. The compound of claim 93, wherein R¹ is other than phenyl, substituted phenyl or methyl.

95. The compound of claim 93, wherein R¹ is other than lower alkyl.

96. The compound of claim 93, wherein R¹ is substituted or unsubstituted heteroalkyl, substituted or unsubstituted acyl, substituted or unsubstituted heteroaryl, substituted or unsubstituted aralkyl, or substituted or unsubstituted heteroaralkyl.

97. The compound of claim 93, wherein R² is other than hydrogen.

98. The compound of claim 93, wherein R² is substituted or unsubstituted (C₁-C₆)alkyl, substituted or unsubstituted (C₁-C₆)cycloalkyl, substituted or unsubstituted aryl, or substituted or unsubstituted aralkyl.

99. The compound of claim 93, wherein R³ is other than —OH, —SMe or —S(C₆H₅).

100. The compound of claim 93, wherein R³ is selected from —SO₂NR⁷R⁸, —SO₃R⁹, —CONR⁷R⁸, —NR⁷R⁸, —PO(OR⁹)NR⁷R⁸, —PO(N⁷R⁸)₂, —PO(OR⁹)₂ and —CO₂R⁹.

101. The compound of claim 93, wherein the compound is of the formula: 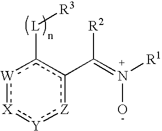
wherein:
W, X, Y and Z are members of a cycloalkenyl, cycloheteroalkenyl, aryl or heteroaryl ring; and
any adjacent pair of W, X, Y and Z are further joined to form, together with the cycloalkenyl, cycloheteroalkenyl, aryl or heteroaryl ring comprising W, X, Y and Z, the bicycloalkenyl, bicycloheteroalkenyl, bicycloaryl or bicycloheteroaryl ring.

102. The compound of claim 101, wherein W and X are further joined to form the bicycloalkenyl, bicycloheteroalkenyl, bicycloaryl or bicycloheteroaryl ring.

103. The compound of claim 101, wherein X and Y are further joined to form the bicycloalkenyl, bicycloheteroalkenyl, bicycloaryl or bicycloheteroaryl ring.

104. The compound of claim 101, wherein Y and Z are further joined to form the bicycloalkenyl, bicycloheteroalkenyl, bicycloaryl or bicycloheteroaryl ring.

105. The compound of claim 93, which is an aryl nitrone compound of formula (V): 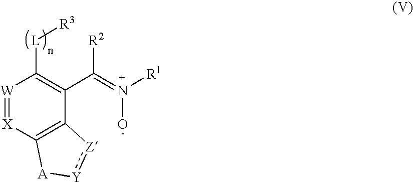
wherein:
W, X and Z′ are each independently C—R⁴ or C(R⁴)₂;
Y is C—R⁴ or carbonyl;
A is selected from NR⁴, O and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents a single or double bond.

106. The aryl nitrone compound of claim 105, wherein X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸S, —PO(OR⁹)₂ and —CO₂R⁹.

107. The aryl nitrone compound of claim 106, wherein R⁵ is not hydrogen.

108. The compound of claim 93, which is an aryl nitrone compound of formula (VI): 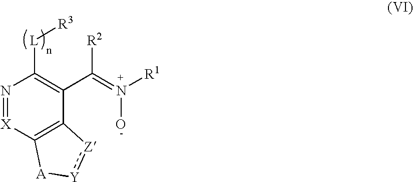
wherein:
X and Z′ are each independently C—R⁴ or C(R⁴)₂;
Y is C—R⁴ or carbonyl;
A is selected from NR⁴, O and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents a single or double bond.

109. The aryl nitrone compound of claim 108, wherein X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

110. The aryl nitrone compound of claim 109, wherein R⁵ is not hydrogen.

111. The compound of claim 93, which is an aryl nitrone compound of formula (VII): 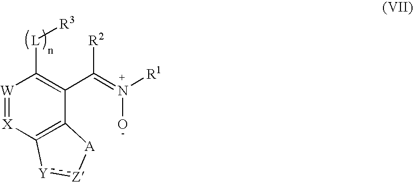
wherein:
W, X and Z′ are each independently C—R⁴ or C(R⁴)₂;
Y is C—R⁴ or carbonyl;
A is selected from NR⁴, O and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents a single or double bond.

112. The aryl nitrone compound of claim 111, wherein X is C—R⁵ and R⁵is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

113. The aryl nitrone compound of claim 112, wherein R⁵ is not hydrogen.

114. The compound of claim 93, which is an aryl nitrone compound of formula (VIII): 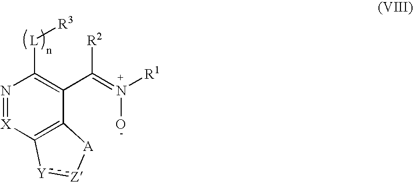
wherein:
X and Z′ are each independently C—R⁴ or C(R⁴)₂;
Y is C—R⁴ or carbonyl;
A is selected from NR⁴, O and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents a single or double bond.

115. The aryl nitrone compound of claim 114, wherein X is C—R⁵ and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

116. The aryl nitrone compound of claim 115, wherein R⁵ is not hydrogen.

117. The compound of claim 93, which is an aryl nitrone compound of formula (IX): 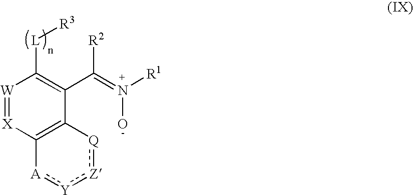
wherein:
W and X are each independently N or C—R⁴;
Y and Z′ are each independently carbonyl, C—R⁴ or C(R⁴)₂;
A and Q are each independently selected from carbonyl, NR⁴, O, S and C—R⁴;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted lines represent single or double bonds.

118. The aryl nitrone compound of claim 117, wherein at least one of W and X is N.

119. The compound of claim 93, which is an aryl nitrone compound of formula (X): 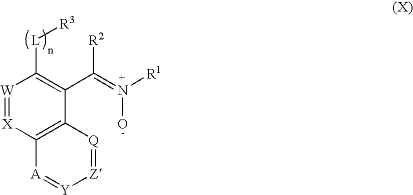
wherein:
W, X, Y, Z′, A and Q are each independently selected from N and C—R⁴; and
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio.

120. The aryl nitrone compound of claim 119, wherein W, X, Y and Z′ are each independently C—R⁴ and R² is other than hydrogen.

121. The aryl nitrone compound of claim 119, wherein W, X, Y and Z′ are each independently C—R⁴ and R¹ is other than methyl, phenyl or substituted phenyl.

122. The aryl nitrone compound of claim 119, wherein W, X, Y and Z′ are each independently C—R⁴ and R¹ is other than lower alkyl, phenyl or substituted phenyl.

123. The aryl nitrone compound of claim 119, wherein W, X, Y and Z′ are each independently C—R⁴ and R¹ is substituted or unsubstituted heteroalkyl, substituted or unsubstituted acyl, substituted or unsubstituted heteroaryl, substituted or unsubstituted aralkyl, or substituted or unsubstituted heteroaralkyl.

124. The aryl nitrone compound of claim 119, wherein W, X, Y and Z′ are each independently C—R⁴ and R³is other than —OH or —SMe.

125. The aryl nitrone compound of claim 119, wherein R³ is selected from —SO₂NR⁷R⁸, —SO₃R⁹, —CONR⁷R⁸, —NR⁷R⁸, —PO(OR⁹)NR⁷R⁸, —PO(NR⁷R⁸)₂, —PO(NR⁷R⁹)₂ and —CO₂R⁹.

126. The aryl nitrone compound of claim 119, wherein W, X, Y, Z′, A and Q are each independently C—R⁴.

127. The aryl nitrone compound of claim 119, wherein W, X, Y, Z′, A and Q are each independently C—R⁴, X is C—R⁵, and R⁵ is selected from hydrogen, —SR⁹, —SO₂NR⁷R⁸, —SO₃R⁹, —CONR⁷R⁸, —NR⁷R⁸, —OH, —PO(OR⁹)NR⁷R, —PO(OR⁹)₂ and —CO₂R⁹.

128. The aryl nitrone compound of claim 127, wherein R⁵ is not hydrogen.

129. The aryl nitrone compound of claim 119, wherein:
W and X are each independently C—R⁴; and
one of Y, Z′, A and Q is N and the remainder are each independently C—R⁴.

130. The aryl nitrone compound of claim 119, wherein A is N, and W, X, Y, Z′ and Q are each independently C—R⁴.

131. The aryl nitrone compound of claim 119, wherein:
W and X are each independently C—R⁴; and
two of Y, Z′, A and Q are N and the remainder are each independently C—R⁴.

132. The aryl nitrone compound of claim 119, wherein W is N, and X, Y, Z′, A and Q are each independently C—R⁴.

133. The aryl nitrone compound of claim 119, wherein:
W is N;
X is C—R⁴; and
one of Y, Z′, A and Q is N and the remainder are each independently C—R⁴.

134. The aryl nitrone compound of claim 119, wherein W and A are each N, and X, Y, Z′ and Q are each independently C—R⁴.

135. The aryl nitrone compound of claim 119, wherein:
W is N;
X is C—R⁴; and
two of Y, Z′, A and Q are N and the remainder are each independently C—R⁴.

136. The aryl nitrone compound of claim 119, wherein X is N, and W, Y, Z′, A and Q are each independently C—R⁴.

137. The aryl nitrone compound of claim 119, wherein:
X is N;
W is C—R⁴; and
one of Y, Z′, A and Q is N and the remainder are each independently C—R⁴.

138. The aryl nitrone compound of claim 119, wherein X and A are each N, and W, Y, Z′ and Q are each independently C—R⁴.

139. The aryl nitrone compound of claim 119, wherein:
X is N;
W is C—R⁴; and
two of Y, Z′, A and Q are N and the remainder are each independently C—R⁴.

140. The compound of claim 93, which is an aryl nitrone compound of formula (XI): 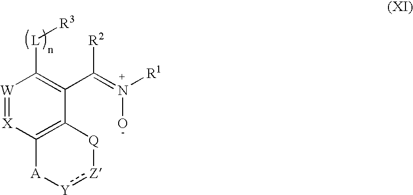
wherein:
W and X are each independently selected from N and C—R⁴;
Y and Z′ are each independently carbonyl, C—R⁴ or C(R⁴)₂;
A and Q are each independently selected from carbonyl, NR⁴, O and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents a single or double bond.

141. The compound of claim 93, which is an aryl nitrone compound of formula (XII): 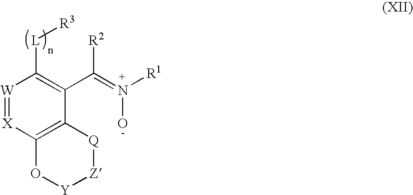
wherein:
W and X are each independently selected from N and C—R⁴;
Q is NR⁴, carbonyl or C(R⁴)₂;
Y and Z′ are each independently C(R⁴)₂ or carbonyl; and
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio.

142. The compound of claim 93, which is an aryl nitrone compound of formula (XIII): 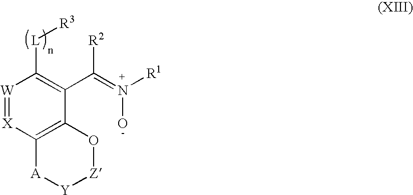
wherein:
W and X are each independently selected from N and C—R⁴;
A is NR⁴, carbonyl or C(R⁴)₂;
Y and Z′ are each independently C(R⁴)₂ or carbonyl; and
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio.

143. The compound of claim 93, which is an aryl nitrone compound of formula (XIV): 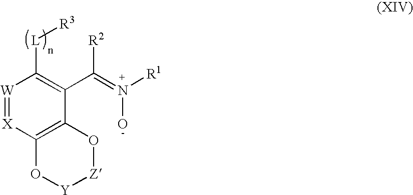
wherein:
W and X are each independently selected from N and C—R⁴;
Y and Z′ are each independently C(R⁴)₂ or carbonyl; and
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sulfonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio.

144. A pharmaceutical composition comprising a pharmaceutically acceptable carrier and a pharmaceutically effective amount of a compound of claim 1, 18 or 93.

145. The pharmaceutical composition of claim 144, wherein the carrier is a parenteral carrier.

146. The pharmaceutical composition of claim 144, wherein the carrier is an oral carrier.

147. The pharmaceutical composition of claim 144, wherein the carrier is a topical carrier.

148. A pharmaceutical composition comprising a pharmaceutically acceptable carrier and a pharmaceutically effective amount of one or more of: compounds 1, 8, 9, 11, 16-22, 25-27, 37-43 and 45-50 in Section 5.3; compounds 51 and 53-69 in Section 5.4; and compounds 70-78 in Section 5.5.

149. The pharmaceutical composition of claim 148, wherein the carrier is a parenteral carrier.

150. The pharmaceutical composition of claim 148, wherein the carrier is an oral carrier.

151. The pharmaceutical composition of claim 148, wherein the carrier is a topical carrier.

152. A method for preventing, treating or ameliorating in a mammal a disease or condition which comprises administering to the mammal an effective disease- or condition-preventing, treating or ameliorating amount of a pharmaceutical composition of claim 144.

153. The method of claim 152, wherein the disease or condition is a pain condition.

154. The method of claim 152, wherein the disease or condition is an autoimmune disease or condition.

155. The method of claim 152, wherein the disease or condition is an inflammatory disease or condition.

156. The method of claim 152, wherein the disease or condition is a neurological or neurodegenerative disease or condition.

157. A method for preventing, treating or ameliorating in a mammal a disease or condition from:
pain including acute, inflammatory and neuropathic pain, chronic pain, dental pain and headache including migraine, cluster headache and tension headache; Parkinson's disease, Alzheimer's disease and multiple sclerosis; diseases and disorders which are mediated by or result in neuroinflammation, traumatic brain injury, stroke, and encephalitis; centrally-mediated neuropsychiatric diseases and disorders, depression, mania, bipolar disease, anxiety and schizophrenia; eating disorders, sleep disorders and cognition disorders; epilepsy and seizure disorders; prostate, bladder and bowel dysfunction, urinary incontinence, urinary hesitancy, rectal hypersensitivity, fecal incontinence, benign prostatic hypertrophy and inflammatory bowel disease;
respiratory and airway diseases and disorders, allergic rhinitis, asthma, reactive airway disease and chronic obstructive pulmonary disease; diseases and disorders which are mediated by or result in inflammation, arthritis, rheumatoid arthritis, osteoarthritis, myocardial infarction, various autoimmune diseases and disorders, uveitis and atherosclerosis; itch/pruritus and psoriasis; alopecia (hair loss); obesity; lipid disorders; cancer; high blood pressure; spinal cord injury; and renal disorders, which comprises administering to the mammal an effective disease- or condition-preventing, treating or ameliorating amount of a pharmaceutical composition of claim 144.

158. The method of claim 157, wherein the disease or condition is Parkinson's disease.

159. The method of claim 157, wherein the disease or condition is Alzheimer's disease.

160. The method of claim 157, wherein the disease or condition is traumatic brain injury.

161. The method of claim 157, wherein the disease or condition is stroke.

162. A method for preventing, treating or ameliorating in a mammal a disease or condition which comprises administering to the mammal an effective disease- or condition-preventing, treating or ameliorating amount of a pharmaceutical composition of claim 148.

163. The method of claim 162, wherein the disease or condition is a pain condition.

164. The method of claim 162, wherein the disease or condition is an autoimmune disease or condition.

165. The method of claim 162, wherein the disease or condition is an inflammatory disease or condition.

166. The method of claim 162, wherein the disease or condition is a neurological or neurodegenerative disease or condition.

167. A method for preventing, treating or ameliorating in a mammal a disease or condition from:
pain including acute, inflammatory and neuropathic pain, chronic pain, dental pain and headache including migraine, cluster headache and tension headache; Parkinson's disease, Alzheimer's disease and multiple sclerosis; diseases and disorders which are mediated by or result in neuroinflammation, traumatic brain injury, stroke, and encephalitis; centrally-mediated neuropsychiatric diseases and disorders, depression, mania, bipolar disease, anxiety and schizophrenia; eating disorders, sleep disorders and cognition disorders; epilepsy and seizure disorders; prostate, bladder and bowel dysfunction, urinary incontinence, urinary hesitancy, rectal hypersensitivity, fecal incontinence, benign prostatic hypertrophy and inflammatory bowel disease; respiratory and airway diseases and disorders, allergic rhinitis, asthma, reactive airway disease and chronic obstructive pulmonary disease; diseases and disorders which are mediated by or result in inflammation, arthritis, rheumatoid arthritis, osteoarthritis, myocardial infarction, various autoimmune diseases and disorders, uveitis and atherosclerosis; itch/pruritus and psoriasis; alopecia (hair loss); obesity;
lipid disorders; cancer; high blood pressure; spinal cord injury; and renal disorders, which comprises administering to the mammal an effective disease- or condition-preventing, treating or ameliorating amount of a pharmaceutical composition of claim 148.

168. The method of claim 167, wherein the disease or condition is Parkinson's disease.

169. The method of claim 167, wherein the disease or condition is Alzheimer's disease.

170. The method of claim 167, wherein the disease or condition is traumatic brain injury.

171. The method of claim 167, wherein the disease or condition is stroke.

172. A method for preparing a nitrone compound of claim 1, 18 or 93, which comprises reacting an aldehyde or ketone compound of the formula: 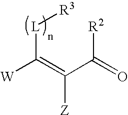
with R¹NHOH or a salt thereof under conditions suitable for preparing the compound.
